(19) United States
(12) Patent Application Publication
(10) Pub. No.: US 2026/0042867 A1
(43) Pub. Date: Feb. 12, 2026

(54) ANTIBODY REPURPOSING

(71) Applicant: SRI International, Menlo Park, CA (US)

(72) Inventors: Michal Avital-Shmilovici, Menlo Park, CA (US); Peter Madrid, Menlo Park, CA (US); Nathan Collins, Menlo Park, CA (US)

(73) Assignee: SRI International, Menlo Park, CA (US)

(21) Appl. No.: 18/852,749

(22) PCT Filed: Jun. 6, 2023
(86) PCT No.: PCT/US2023/067956

§ 371 Date: Sep. 30, 2024

Related U.S. Application Data
(60) Provisional application No. 63/349,411, filed on Jun. 6, 2022.

(51) Int. Cl. C07K 16/46; C07K 16/10; C07K 16/42

(52) U.S. Cl. CPC C07K 16/468; C07K 16/1003; C07K 16/4208; C07K 2317/31; C07K 2317/92

(57) ABSTRACT

The disclosure relates to compounds and methods for repurposing antibodies.

### CROSS-REFERENCE TO RELATED APPLICATIONS

This application claims the benefits of priority from U.S. Provisional Application No. 63/349,411, filed Jun. 6, 2022.

### REFERENCE TO A “SEQUENCE LISTING” SUBMITTED ELECTRONICALLY

The instant application contains a Sequence Listing, which is labeled SRI-012_P190026_SL.xml, and which has been submitted electronically in XML format and is hereby incorporated by reference in its entirety. Said .XML copy is 76,415 bytes in size.

### BACKGROUND OF THE DISCLOSURE

Discovery and development of antibodies (“Abs”) for therapeutic applications take years to identify engineered Abs with target binding and selectivity as well as clinical safety and efficacy. However, once identified, Abs have been shown to have exquisite target selectivity, and have generally clean and safe therapeutic windows and long durations of action (up to 24 days) in humans. The antibody drug discovery process is not as well suited when a rapid response to a new biological or chemical threat is needed; e.g., in response to bioterrorism agents, chemical or biological weapons, or global pandemics.

Currently, no platforms are available for the discovery, development, and manufacture of therapeutic Abs on a time scale suitable for rapid response to novel chemical and biological threats. Alternative treatment options primarily focus on quarantine, either through hospitalization or quarantine of infected locations to prevent spread of the threat agent. Thus, there is a need for rapid discovery and development of Abs to new threats from target identification to a field-ready therapeutic agent in approximately 6 months or less. In addition, the ability to repurpose an antibody from recognizing an original target to recognizing a second/new target opens up numerous possibilities for treatment of any disorder beyond infections, including cancer.

### SUMMARY OF THE DISCLOSURE

Disclosed herein are new synthetic compounds and methods of using those compounds for repurposing original antibodies originally directed to a first target so that they may be re-directed to a second, novel target without structural changes to the original antibody. The methods rely on the conjugation of original antibodies with target re-directing synthetic compounds, which comprise at least two molecules covalently bound to each other directly or through a linker, a first molecule being capable of non-covalently binding the variable region of the original antibody, which binds to a first target, and a second molecule being capable of and non-covalently binding a different, second target.

In one embodiment, the first and/or second molecules are synthetic polymers also referred to herein as “Techneins.” The Techneins disclosed herein are novel molecules, not previously described elsewhere, although several other Techneins have been previously described in the art, such as in PCT/US17/50119 entitled, “MASS SPECTROMETRY DISTINGUISHABLE SYNTHETIC COMPOUNDS, LIBRARIES, AND METHODS THEREOF”, PCT/US15/50306 entitled, “Affinity Reagent and Catalyst Discovery Though Fiber-Optic Array Scanning Technology”, and in PCT/US21/55226 entitled, “HIGH AFFINITY NON-NATURAL LIGANDS AGAINST PROTEIN TARGETS,”, the contents of which are incorporated by reference in their entirety.

In one embodiment, one may synthetically optimize the first and second molecules or affinity agents (e.g., Technein) for their target (antibody or novel target), as well as optimize absorption, distribution, metabolism, excretion, and toxicity (ADMET) profile as well as pharmacokinetics and dynamics (PK/D). It has been demonstrated that Techneins have remarkable stability and limited toxicity effects in human tissues.

In one embodiment, identified and optimized Techneins may be conjugated to each other, directly or through a linker. In one embodiment, the Techneins may be conjugated with a tight binding epitope for a previously FDA approved Ab with desired adaptive immune recruiting properties (i.e., that recruit macrophages to engage and neutralize bound target proteins) and a proven track record of safety in human use. Co-administration of a Technein-epitope conjugate to a second target with an Ab which typically binds to a first target will now cause the Ab to bind the second target through binding to the epitope that is conjugated to the Technein that is bound to the second target. In so doing the approved Ab is repurposed for binding and immunological recruitment of neutralizing factors against the second target. In some embodiments, the repurposed antibody works via various effector functions of the Fc region of an antibody (ADCC, ADCP, complement activation, and interactions with other receptors on cells like Fc-receptor and the like).

The following embodiments are exemplary, but not limited, embodiments of the disclosure, which are numbered sequentially:

1. A synthetic compound comprising at least one unit, each unit comprising:
(i) a first molecule that binds to an antibody, or to a target-binding fragment thereof; wherein the antibody originally binds to a first target; and
(ii) a second molecule that binds to a second target;
wherein the first target and the second target are different, and wherein the first molecule and the second molecule are covalently linked, directly or through a linker.
   2. The synthetic compound of embodiment 1, wherein the first molecule comprises the first target, an epitope thereof, an antibody-binding fragment thereof, or a first synthetic polymer; and the second molecule comprises a second synthetic polymer that binds to a second target, wherein the first and second synthetic polymers comprise amino acid monomers and/or non-amino acid monomers.
   3. The synthetic compound of embodiment 1, wherein the first molecule non-covalently binds to one or more complementarity determining regions (CDRs) of the antibody.
   4. The synthetic compound of embodiment 1, wherein the antibody has been approved by one or more agencies selected from Federal Drug Administration (FDA), European Medicines Agency (EMA), SwissMedic, China Food and Drug Administration (CFDA), and Japanese Pharmaceuticals and Medical Devices Agency (PMDA).
   5. The synthetic compound of embodiment 4, wherein the antibody is selected from Trastuzumab (Herceptin), Adalimumab (Humira), Bevacizumab (Avastin), Rituximab (Rituxan/MabThera), Infliximab (Remicade), or any other antibody from Table 1.
   6. The synthetic compound of embodiment 1, wherein the antibody is an endogenous antibody.
   7. The synthetic compound of embodiment 6, wherein the first target and the first molecule are both alpha-Gal.
   8. The synthetic compound of embodiment 1, wherein the targets are selected from proteins, sugars, carbohydrates, nucleic acids, lipids, and combinations thereof.
   9. The synthetic compound of embodiment 1, wherein the targets are viral, tumor-specific (e.g., tumor-associated antigens), tissue-specific, cell-specific, bacterial, fungal, and combinations thereof.
   10. The synthetic compound of embodiment 1, wherein the first target is Human Epidermal growth factor Receptor 2 (HER2) and/or the second target is a viral protein (including, but not limited to SARS-CoV-2 spike protein).
   11. The synthetic compound of embodiment 2, wherein the amino acids comprise L-amino acids, D-amino acids, beta-amino acids, gamma-amino acids, or combinations thereof, and the non-amino acid monomers comprise PAM, DhqF, DhqB, DhqO, DhqY, DhqE, N-substituted glycines, triazines, pyrimidines, or combinations thereof.
   12. The synthetic compound of embodiment 1, wherein the linker comprises Polyethylene glycol (PEG), poly-glycine sequences, peptides, alkyl chains, cysteines, lysines, glutamines, maleimides, dibenzyclooctanes, or combinations thereof.
   13. The synthetic compound of embodiment 1, wherein at least one unit comprises multiple first and/or second molecules, wherein:
i. the multiple first molecules are covalently bound to each other directly or through a linker; and/or
ii. the multiple second molecules are covalently bound to each other directly or through a linker;
preferably, wherein the linker(s) comprise PEG, Lys, Gln, or combinations thereof.
   14. The synthetic compound of embodiment 1, wherein the first molecule and/or second molecule binds to the antibody or second target, respectively, with an affinity below 500 nM, preferably below 200 nM, more preferably below 100 nM.
   15. The synthetic compound of embodiment 1, wherein the first molecule and/or second molecule does not bind significantly to any other target, also referred to as being specific for the first target or second target, respectively.
   16. A conjugate comprising an antibody, or antigen-binding fragment thereof, non-covalently bound to at least one synthetic compound of any one of embodiments 1 to 15, wherein the conjugate is also referred to as a repurposed antibody, or repurposed fragment thereof.
   17. The conjugate of embodiment 16, wherein the antibody comprises a light chain and a heavy chain immunoglobulin variable region and constant regions.
   18. The conjugate of embodiment 16, wherein the antibody, or antigen-binding fragment thereof, is selected from monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab, Fab′, F(ab′)2, and Fv fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments of any of the above.
   19. The conjugate of any one of embodiments 16 to 18 and/or synthetic compound of any one of embodiments 1 to 15, wherein the antibody that binds to the first molecule, the conjugate (repurposed antibody), and/or the synthetic compound is an immunotherapeutic agent.
   20. A method of repurposing an antibody that binds to a first target, or a first target-binding fragment thereof, by changing its target from a first target to a second target, the method comprising non-covalently conjugating the antibody, or first target-binding fragment thereof, with at least one synthetic compound of any one of embodiments 1 to 15, wherein the second target is also referred to as the repurposed target and wherein the conjugate is also referred to as the repurposed antibody.
   21. A method of treating a disorder with a repurposed antibody in a subject in need thereof, comprising administering to the subject an effective amount of the synthetic compound of embodiments 1 to 15 (or a part thereof), the antibody of any one of embodiments 1 to 15, and/or the repurposed antibody of anyone of embodiments 16 to 19.
   22. The method of embodiment 21, wherein the disorder is an infectious disorder caused by a pathogen that comprises the second target or a cancer expressing the second target.
   23. The method of embodiment 22, wherein the pathogen is a virus (including, without limitation, the SARS-COV-2 coronavirus).
   24. The method of any one of embodiments 20 to 23, wherein the antibody is an endogenous antibody.
   25. A method for treating a disease caused by a pathogen in a subject in need thereof comprising:
(i) administering to the subject an antibody that binds to the first molecule of a synthetic compound according to any one of embodiments 1 through 15, or confirming that the subject carries an endogenous antibody that binds to the said compound;
(ii) exposing or having exposed the subject to a pathogen, an epitope of the pathogen, and/or a part of the pathogen; and
(iii) administering the synthetic compound of (i) to the subject, wherein the pathogen comprises the second target of the synthetic compound.
   26. The method of embodiment 25, wherein the pathogen causes a disease selected from a viral disease, a bacterial disease, a fungal disease, or a tick-associated disease.
   27. A method of ameliorating, treating, or reducing the incidence of a cancer/tumor/malignancy in a subject in need thereof, preferably, wherein the method assists or boost the immune system in killing or eradicating cancerous cells, wherein the method comprises administering a therapeutic effective amount of one or more conjugates/repurposed antibodies, synthetic compounds, or antibodies of any one of embodiments 1 to 19 to the subject, preferably one which invokes an active (or achieve a passive) immune response to destroy cancerous cells.
   28. A method of ameliorating, treating, or reducing the incidence of a cancer/tumor/malignancy in a subject in need thereof, preferably, wherein the method assists or boost the immune system in killing or eradicating cancerous cells, wherein the method comprises administering to the subject a therapeutic effect amount of a CAR-T cell, T cell, and/or Tumor Infiltrating Lymphocyte contacted with or bound to an immunotherapeutic compound of any one of embodiments 1 to 19, preferably, wherein the cells invoke an active (or achieve a passive) immune response to destroy cancerous cells.
   29. A method of using a therapeutic effective amount of conjugates/repurposed antibodies, synthetic compounds, or antibodies, of any one of claims 1 to 19, or immunotherapeutic cells (including using autologous and/or heterologous cells or immortalized cell lines) conjugated or contacted with a compound or conjugate of any one of embodiments 1 to 19, in vivo or ex vivo as an adoptive immunotherapies for treating a cancer, in a subject in need thereof, comprising using or administering one or more conjugates/repurposed antibodies, synthetic compounds, antibodies of any one of embodiments 1 to 19 to a subject and/or contacting the immunotherapeutic cells with one or more conjugates/repurposed antibodies, synthetic compounds, antibodies cells, and/or nucleic acids of any one of embodiments 1 to 19.
   30. A composition comprising one or more conjugates/repurposed antibodies, synthetic compounds, and/or antibodies of one of embodiments 1 to 19, and/or CAR-T cells, T cells, or TILs contacted with or conjugated with a compound of any one of embodiments 1 to 19, preferably wherein the composition is a pharmaceutical composition.
   31. An immunotherapeutic compound of anyone of embodiments 1 to 19, or composition of embodiment 30, used in a cancer immunotherapy, wherein the immunotherapeutic compound or composition comprises a repurposed antibody, a fragment thereof, and/or synthetic compound of the disclosure, and/or a CAR-T cell, T cell, and/or Tumor Infiltrating Lymphocyte contacted with or conjugated with a compound of any one of embodiments 1 to 19.
   32. Use of any one of the synthetic compounds of any one of embodiments 1 to 15, conjugates of any one of embodiments 16 to 19, compositions comprising the same, and/or CAR-T cells, T cells, or TILs contacted with or conjugated with a compound of any one of embodiments 1 to 19 in the treatment of a disease or disorder.
   33. The use of claim 32, wherein the disease or disorder is an infectious disease or disorder or a cancer, or any other disease disorder described in the specification.
   34. A synthetic compounds of any one of embodiments 1 to 15, conjugates of any one of embodiments 16 to 19, compositions comprising the same, and/or CAR-T cells, T cells, or TILs contacted with or conjugated with a compound of any one of embodiments 1 to 19 for use in the manufacture of a medicament in the treatment of a disease or disorder, preferably, wherein the disease or disorder is an infectious disease or disorder or a cancer, or any other disease disorder described in the specification.

### BRIEF DESCRIPTION OF DRAWINGS

FIG. 1: Antibody Repurposing example.

FIG. 2A and FIG. 2B examples of “second molecules” comprising synthetic polymers: Technein hits against spike receptor binding domain (SRBD) SARS-COV-2 (16838-89-XX); FIG. 2C: Technein hits against SRBD Sars-Cov-2-biotinylated hits; FIG. 2D: Building blocks of hits (all hits are from DHQ10 library).

FIG. 3A (examples of “first molecules” comprising synthetic polymers): Technein hits (from DAAP2 library) against Herceptin (16989-3-XX) (examples of “first molecules” comprising synthetic polymers); FIG. 3B: Technein hits (from DAAP2 library) against Herceptin (16910-59-XX) and Technein hits (from DHQ11 library) against Herceptin (16989-11-XX) (examples of “first molecules” comprising synthetic polymers); FIG. 3C: Building blocks of hits from DAAP2 library; FIG. 3D: Building blocks of hits from DHQ11 library.

FIG. 4: Repurposing Agents for Anti-Biotin Antibody Against Spike Protein (Technein-linker-Biotin)

FIG. 5A, 5B, 5C, 5D. Repurposing Agents for Anti-alphaGal Antibody Against Spike Protein.

FIG. 6A, 6B: Repurposing Agents for Anti-Herceptin Antibody Against Spike Protein.

FIG. 7: Biotin antibody was repurposed to bind SARS-2 spike protein.

FIG. 8: Biotin antibody promotes immune response in cell-based functional assay for clearing SARS-2 spike protein by ADCP (Antibody-Dependent Cellular Phagocytosis).

FIG. 9: Repurposing of Herceptin (example of an FDA-approved antibody drug).

FIG. 10: Repurposing of α-Gal Antibody (example of an endogenous antibody).

FIG. 11: Binding to both anti-alpha-Gal antibody and SARS-Cov2 antigens (SARS-Cov2 SRBD or Omicron S1/S2 ECD) by the antibody repurposing Techneins in a sandwich enzyme-linked immunosorbent assay (ELISA).

FIG. 12A, 12B, 12C: ADCP assay with the repurposing agent alpha Gal-89-8. Total phagocytic score was determined by the percentage of cells with beads, multiplied by their MFI. FIGS. 12A and 12B: phagocytic scores; FIG. 12C: antigen-specific phagocytic score.

FIG. 13A: Spike-binding by Technein monomer vs. dimer; FIG. 13B: Technein dimer, two identical Techneins each bound to its own PEG molecule, which then converges into a single PEG via a short linker. The latter PEG binds biotin.

FIG. 14: Example of application of antibody repurposing for vaccination against infectious agent.

### DETAILED DESCRIPTION OF THE DISCLOSURE

### Definitions

In order for the present disclosure to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.,” as used herein, are used merely by way of example, without limitation intended, and should not be construed as referring only those items explicitly enumerated in the specification.

The terms “or more,” “at least,” “more than,” and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000, or more than the stated value. Also included is any greater number or fraction in between.

Conversely, the term “no more than” includes each value less than the stated value. In one embodiment, “no more than 100 monomers” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 monomers. Also included is any lesser number or fraction in between.

The terms “plurality,” “at least two,” “two or more,” “at least second,” and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000, or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided. The term “consisting of” excludes any element, step, or ingredient not specified in the claim. In re Gray, 53 F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed disclosure.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. In one embodiment, “about” or “approximately” may mean within one or more than one standard deviation per the practice in the art. “About” or “approximately” may mean a range of up to 10% (i.e., ±10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. In one embodiment, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. In one embodiment, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

A “therapeutically effective amount,” “effective dose,” “effective amount,” or “therapeutically effective dosage” of a therapeutic agent, e.g., Technein, repurposed antibody, small molecules, “agents” described in the specification, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. Such terms may be used interchangeably. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays. Therapeutically effective amounts and dosage regimens may be determined empirically by testing in known in vitro or in vivo (e.g., animal model) systems.

In certain embodiments, the “therapeutically effective amount” in the context of a SARS CoV-2 infection is an amount sufficient to reduce one or more of the following steps of a the life cycle of SARS-COV-2: the docking of the virus particle to a cell, the introduction of viral genetic information into a cell, the expression of viral proteins, the translation of viral RNA, the transcription of viral RNA, the replication of viral RNA, the synthesis of new viral RNA, the production of new virus particles and the release of virus particles from a cell. Such a reduction in any of the foregoing may be by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In some embodiments, the “therapeutically effective amount” in the context of a SARS-COV-2 infection reduces the replication, multiplication or spread of the virus by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In some embodiments, the “therapeutically effective amount” in the context of a SARS-COV-2 infection increases the survival rate of infected subjects by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In each of the foregoing, when a reduction of increase is specified, such reduction of increase may be determined with respect to a subject that has not been treated with an antibody of the disclosure and that has a diagnosed SARS-COV-2 infection.

The term “combination” refers to either a fixed combination in one dosage unit form, or a combined administration where a compound of the present disclosure and a combination partner (e.g., another drug as explained below, also referred to as “therapeutic agent” or “agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time.

A “patient” or a “subject” as used herein includes any human who is afflicted with a heart disease or disorder. The terms “subject” and “patient” are used interchangeably herein.

As used herein, the term “epitope” refers to an antigenic determinant that interacts with (is bound by) a specific antigen binding site in the variable region of an antibody molecule (the paratope). A single antigen (such as, but not limited to, a polypeptide) may have more than one epitope. Thus, different antibodies may bind to different epitopes on an antigen and may have different biological effects depending on which epitope is bound. The term “epitope” also refers to a site on an antigen to which B and/or T cells respond. It also refers to a region of an antigen that is bound by an antibody. Epitopes may be defined as a structural epitope (the portion of the antigenic determinant that is contacted by the CDR loops of an antibody) or a functional epitope (a subset of a structural epitope comprising those energetic residues centrally located in the structural epitope and directly contribute to the affinity of the antibody-epitope interaction). Epitopes may become immunologically available after fragmentation or denaturation of an antigen (a cryptotope). Epitopes may be linear or conformational (composed of non-linear amino acids brought together in a folded three-dimensional structure). Epitopes may include residues that are chemically active surface groupings of molecules such as amino acids, sugar side chains, phosphoryl groups, or sulfonyl groups, and may have specific three-dimensional structural characteristics, and/or specific charge characteristics. An epitope typically includes at least 3 to 15 amino acids.

The term “antibody” is used herein in the broadest sense and encompasses various antibody structures and antibody fragments so long as they exhibit the desired antigen-binding activity and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins may be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known. Examples of antibody structures include, but are not limited to, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab, Fab′, F(ab′)2, and Fv fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”). In one embodiment, the antibody has been previously approved for clinical use by a regulatory agency.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). A single VH or VL domain may be sufficient to confer antigen-binding specificity. Furthermore, antibodies that bind a particular antigen may be isolated using a VH or VL domain from an antibody that binds to the antigen to screen a library of complementary VL or VH domains, respectively. See, e.g., Portolano et al., J. Immunol. 150:880-887 (1993); Clarkson et al., Nature 352:624-628 (1991).

The term “antigen-binding fragment” refers to a molecule other than an intact antibody, such molecule comprises a portion of the intact antibody and binds to the antigen to which the intact antibody binds. Examples of antigen-binding fragments include, but are not limited to, Fv, Fab, Fab′, Fab′-SH, F(ab′)2, diabodies, dAb, linear antibody, single-chain antibodies (e.g., scFv); single-domain antibodies; antigen-binding fragments of bivalent or bispecific antibodies; camelid antibodies; single domain antibodies, maxibodies, minibodies, nanobodies, intrabodies, diabodies, triabodies, tetrabodies, v-NAR and bis-scFv, and other fragments with desired antigen (e.g., SARS-COV spike)-binding ability.

The term “antigen-binding site” refers to the part of an antibody molecule that comprises determinants that form an interface that binds to a polypeptide, or an epitope thereof. With respect to proteins (or protein mimetics), the antigen-binding site typically includes one or more loops (of at least, e.g., four amino acids or amino acid mimics) that form an interface that binds to a polypeptide. Typically, the antigen-binding site of an antibody molecule includes at least one or two CDRs and/or hypervariable loops, or more typically at least three, four, five or six CDRs and/or hypervariable loops.

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as LI, L2 and L3 or HI, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDR.

As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a KD (affinity) for binding the target of at least about 10-4M, 10-5M, 10-6M, 107M, 10-8M, 10-9M, 10-10M, 10-11M, 10 12M, 10-13M, or less, preferably as measured by the method of the Examples (biolayer interferometry (BLI).

The term “antibody repurposing” refers to the change in the target (binding molecule) of an original antibody from a first/original target into a second/new target. Antibody repurposing may be achieved by conjugation of an original antibody to a synthetic compound, which comprises (i) a first molecule that binds to the variable region of the original antibody and (ii) a second molecule that binds to a second/new target, wherein the first molecule and the second molecule are covalently linked directly or through a linker. The synthetic compound acts as a bridge between the original antibody and its new target, thereby repurposing the antibody towards a new target. The second molecule comprises a synthetic polymer, referred to herein as a Technein.

The term “Technein” refers to a specific subset of synthetic polymers made up of covalently linked monomers that are amino acids and monomers that are not amino acids. The amino acids may be, for example, D-amino acids (e.g., D-Glu(tBu); D-Lys), L-amino acids, beta-amino acids, gamma-amino acids, or combinations thereof. Non-limiting examples of monomers that may be used for Techneins may be found in the figures and include PAM, DhqF, DhqB, DhqO, DhqY, DhqE, N-substituted glycines, triazines, pyrimidines, and combinations thereof.

The term “linker” refers to a chemical moiety comprising a covalent bond or a chain of atoms that covalently attaches one molecule to another (e.g., a Technein to another Technein). In various embodiments, linkers include a divalent radical such as an alkyldiyl, an aryldiyl, a heteroaryldiyl, moieties such as: —(CR2) nO (CR2) n-, repeating units of alkyloxy (e.g., polyethylenoxy, PEG, polymethyleneoxy) and alkylamino (e.g., polyethyleneamino); and diacid ester and amides including succinate, succinamide, diglycolate, malonate, and caproamide. In various embodiments, linkers may comprise one or more amino acid residues, such as valine, phenylalanine, Cys, lysine, and homolysine.

The term “synthetic” is generally used herein to refer to compounds or molecules, e.g., compounds described herein, that are not naturally occurring.

As used herein, the term “polypeptide” refers to polymers of amino acids. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures. In some embodiments, the polypeptide is greater than 50 amino acids in length.

As used herein, “peptide” is less than or equal to 50 amino acids long, e.g., about 5, 10, 15, 20, 25, 30, 35, 40, 45, or 50 amino acids long.

As used herein, the terms “Her-2,” “ErbB2,” “c-Erb-B2,” “HER2,” “Her2,” and “neu” are used interchangeably and refer to native HER2, and allelic variants thereof. Unless indicated otherwise, the terms “HER2,” “ErbB2,” “c-Erb-B2,” “HER2,” and “Her2” when used herein refer to the human protein. The gene encoding Her2 is referred to herein as “ErbB2.

The term “aptamer” refers to biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Aptamers may be peptides. Aptamers may also be nucleic acids. Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and Hicke B J, Stephens A W. “Escort aptamers: a delivery service for diagnosis and therapy.” J Clin Invest 2000, 106:923-928). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4). In some embodiments, “a nucleic acid aptamer” refers to single-stranded or double-stranded oligo-DNA, oligo-RNA or oligo-DNA/RNA or any analogue thereof that specifically binds to a target molecule such as a peptide. Advantageously, aptamers display fairly high specificity and affinity for their targets. Aptamer production is described inter alia in U.S. Pat. No. 5,270,163; Ellington & Szostak 1990 (Nature 346:818-822); Tuerk & Gold 1990 (Science 249:505-510); or “The Aptamer Handbook: Functional Oligonucleotides and Their Applications”, by Klussmann, ed., Wiley-VCH 2006, ISBN 3527310592, incorporated by reference herein. The term “photoaptamer” refers to an aptamer that contains one or more photoreactive functional groups that can covalently bind to or crosslink with a target molecule. The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13:132-134).

The term “affinity” as used herein refers to the strength of the sum of all noncovalent interactions between a single binding site of a molecule (e.g., an antibody) and its binding partner (e.g., an antigen). Unless otherwise indicated, as used herein, “binding affinity” refers to the intrinsic binding affinity which reflects a 1:1 interaction between members of a binding pair (e.g., an antibody and an antigen). The affinity of molecule X for its partner Y is generally expressed by the dissociation constant (KD). Methods for determining binding affinity are known in the art, including surface plasmon resonance (e.g., SPR, BIACORE) or similar techniques (e.g., ForteBio; biolayer interferometry (BLI)).

The term “immunotherapeutic agent” refers to a synthetic compound or repurposed antibody (conjugate) of the disclosure (which can be construed as a immunotherapeutic compound or composition.

### Antibody Repurposing

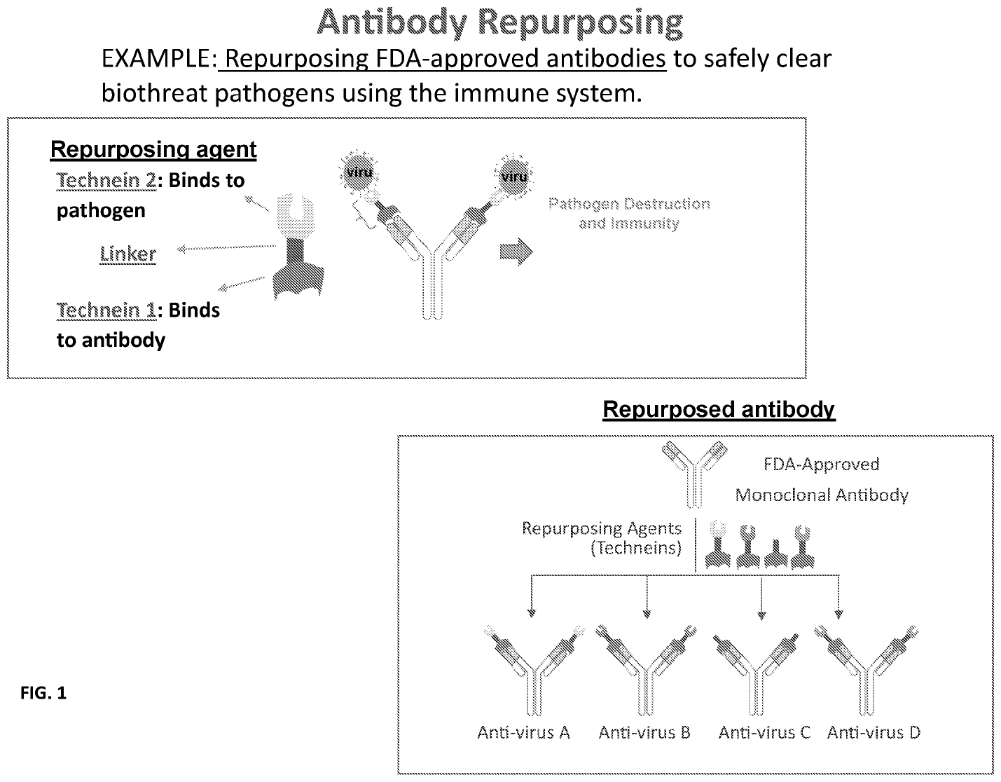

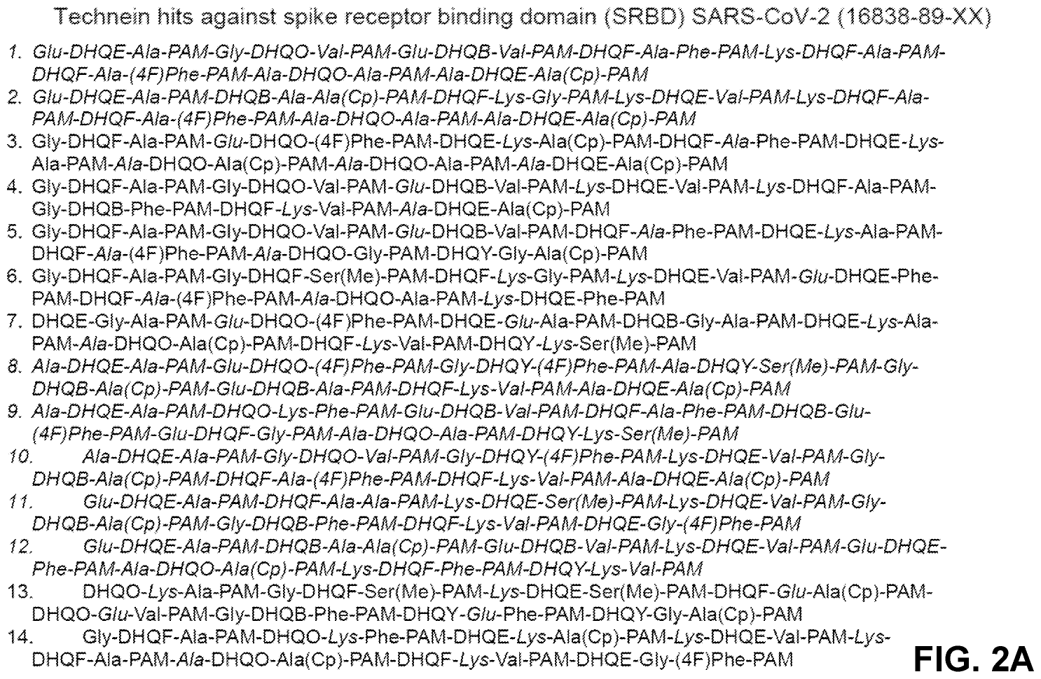

The disclosure relates to a method of repurposing antibodies by changing their target without covalent modifications to their original structure. In one embodiment, this method allows the repurposing of antibodies that have already been approved by health regulatory agencies such as the Food and Drug Administration (FDA), European Medicines Agency (EMA), SwissMedic, China Food and Drug Administration (CFDA), and Japanese Pharmaceuticals and Medical Devices Agency (PMDA) without having to conduct the same degree of extensive clinical testing as that which is applied to newly developed antibodies prior to approval. Antibody repurposing is achieved by altering the target of the antibody. Any antibody has an original target, which is the original molecule which the antibody is designed or produced to bind to, via its complementarity determining regions (CDRs) in its variable region. This disclosure provides a method of altering that original target by non-covalent conjugation of the antibody with a synthetic compound which serves as a bridge between the antibody's variable region (which binds to the original target) and the second, new target. This synthetic compound or bridge is capable of binding the antibody's variable region on one end and the second/new target on the other end. In order to do that, the synthetic compound comprises at least two different molecules, which are linked covalently directly or through a linker. One molecule (“first molecule”) binds to the antibody's variable region. The other molecule (“second molecule”) binds to the second/new target. At least one of these molecules is a synthetic polymer, which, in some embodiments, may be referred to herein as a “Technein.” An antibody repurposing example may be seen in FIG. 1. Antibody repurposing may alter the clinical use of the antibody without covalent modification. In one embodiment, an antibody that is initially approved by health regulatory agencies for its use in cancer treatment may be repurposed for use as a vaccine against an infectious agent. In addition, repurposing of CAR-T cells is also possible by changing their target via the repurposing method disclosed herein.

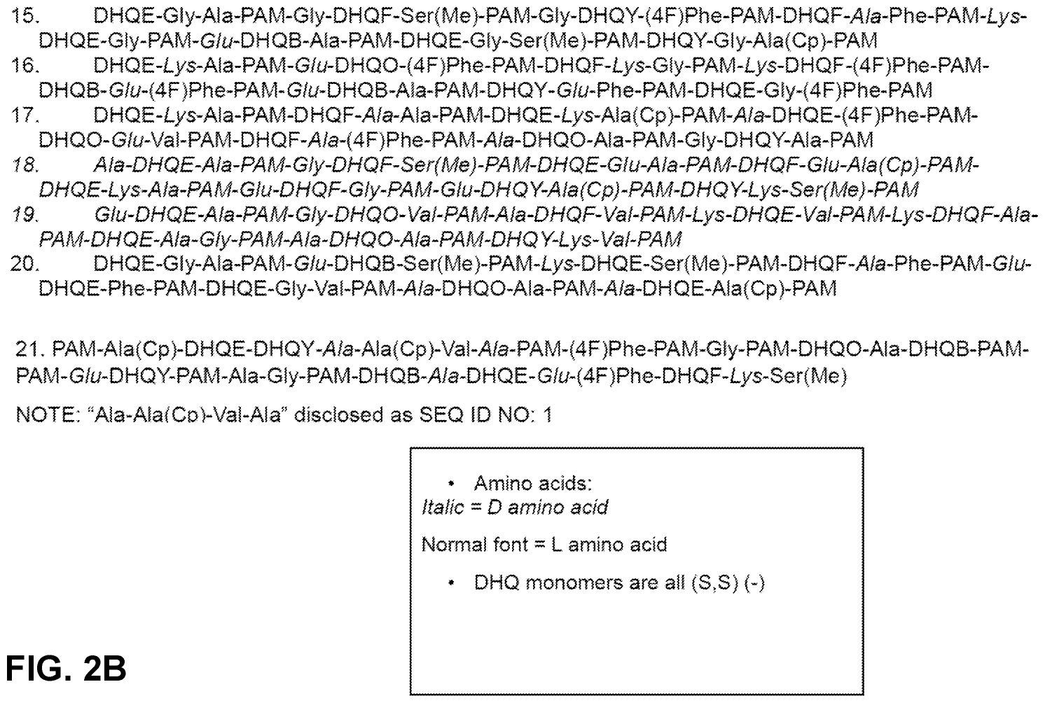

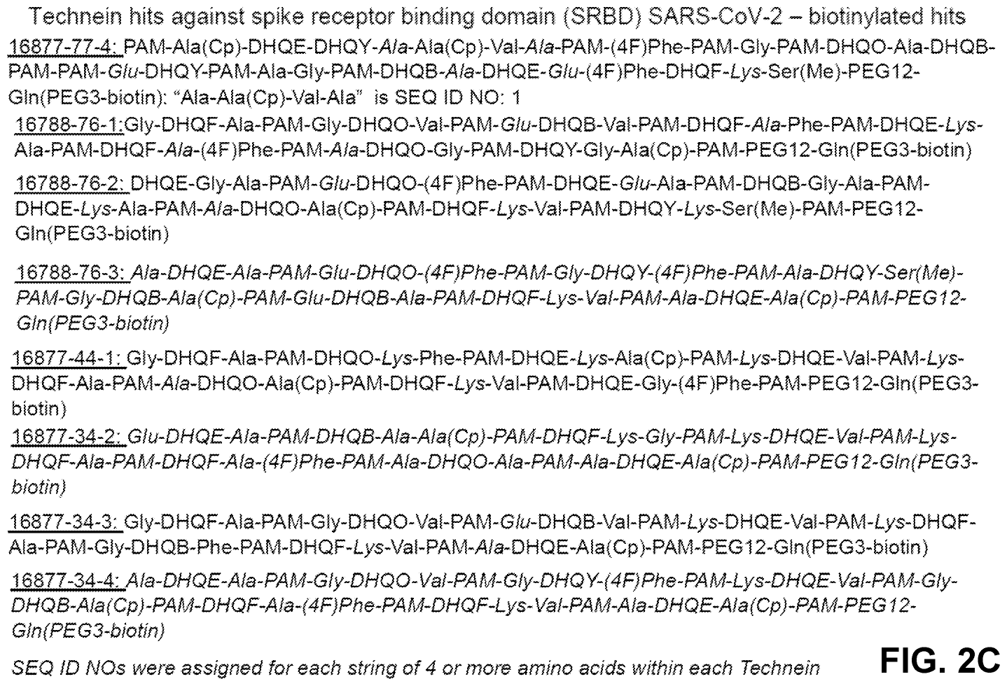

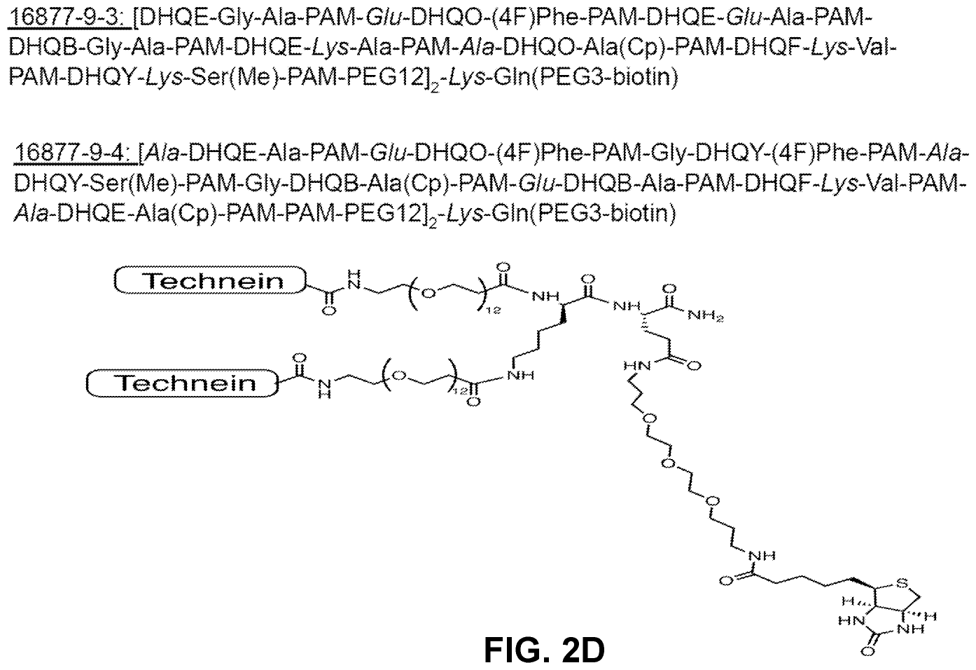

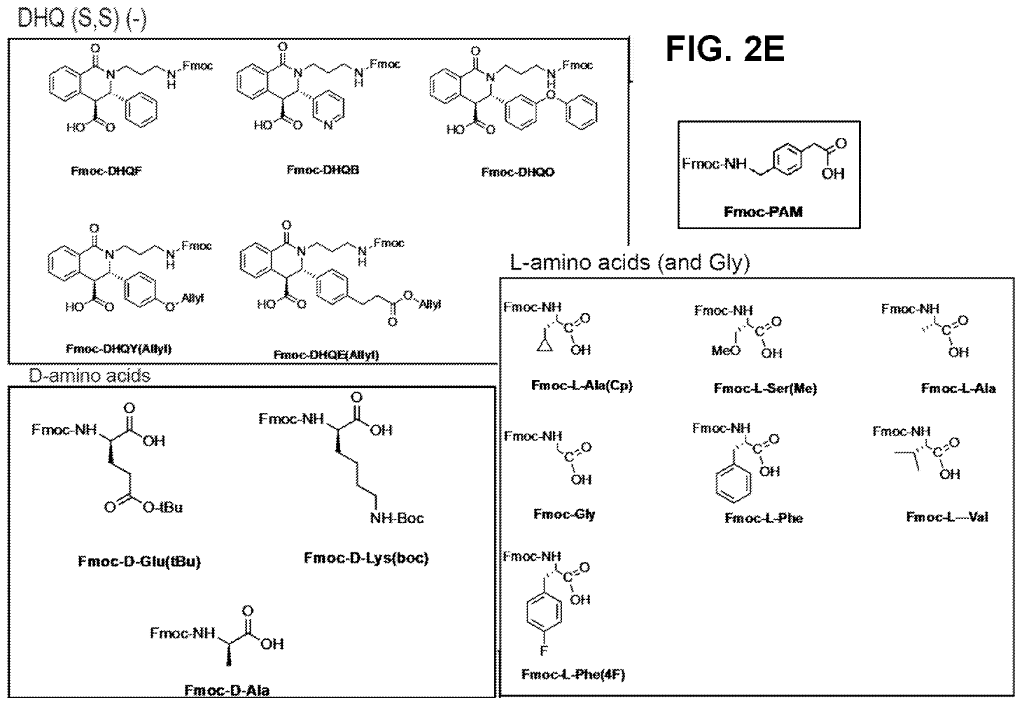

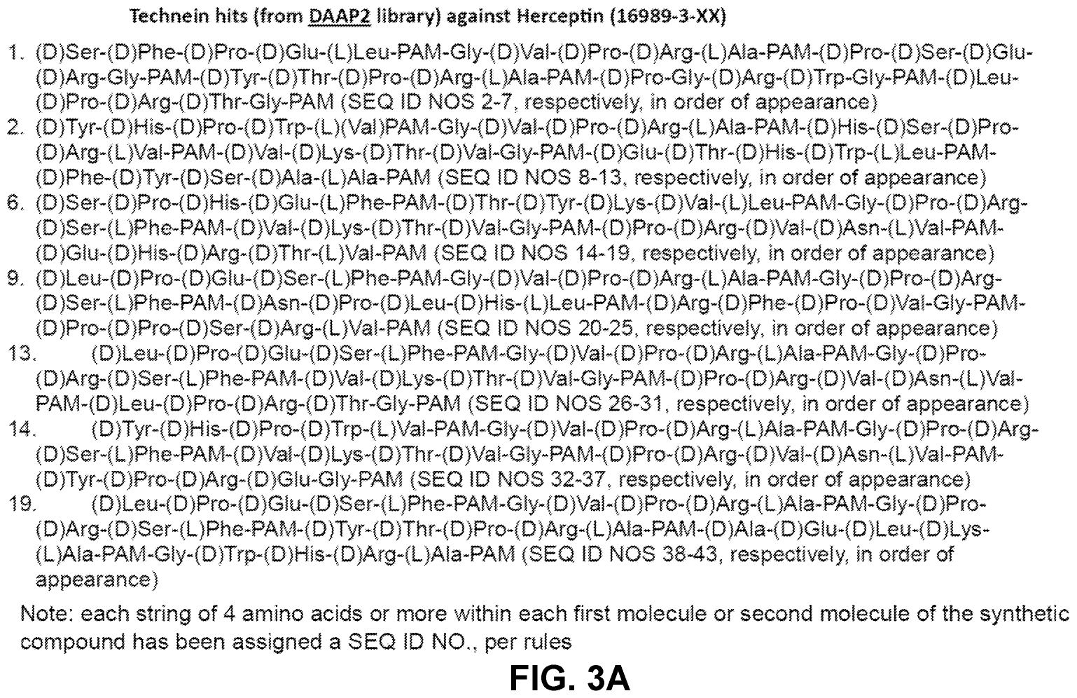

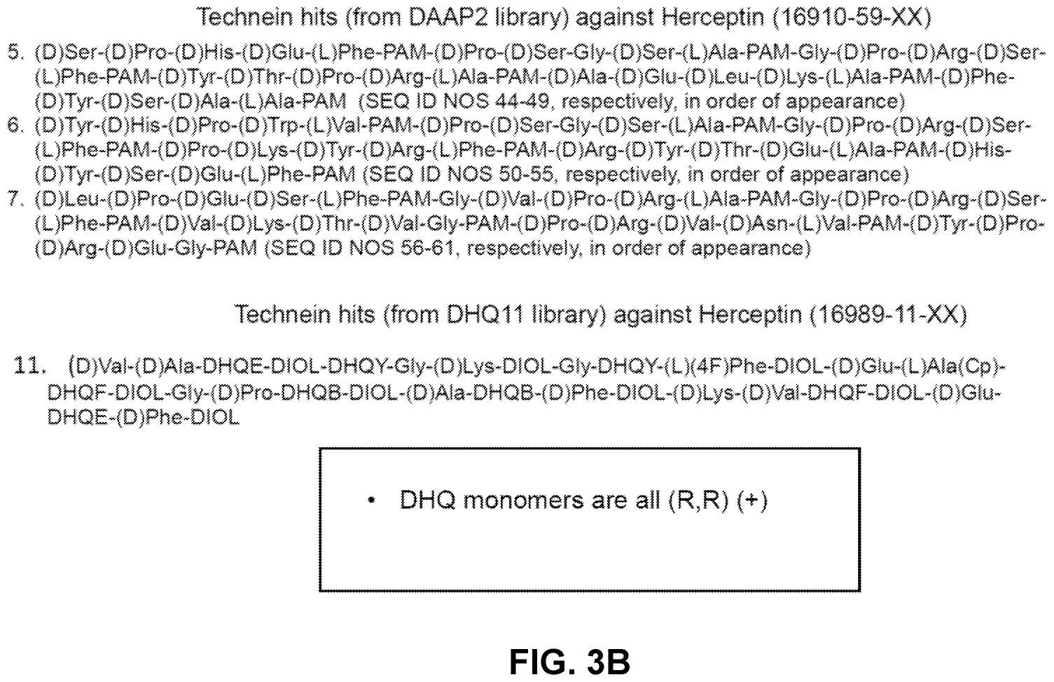

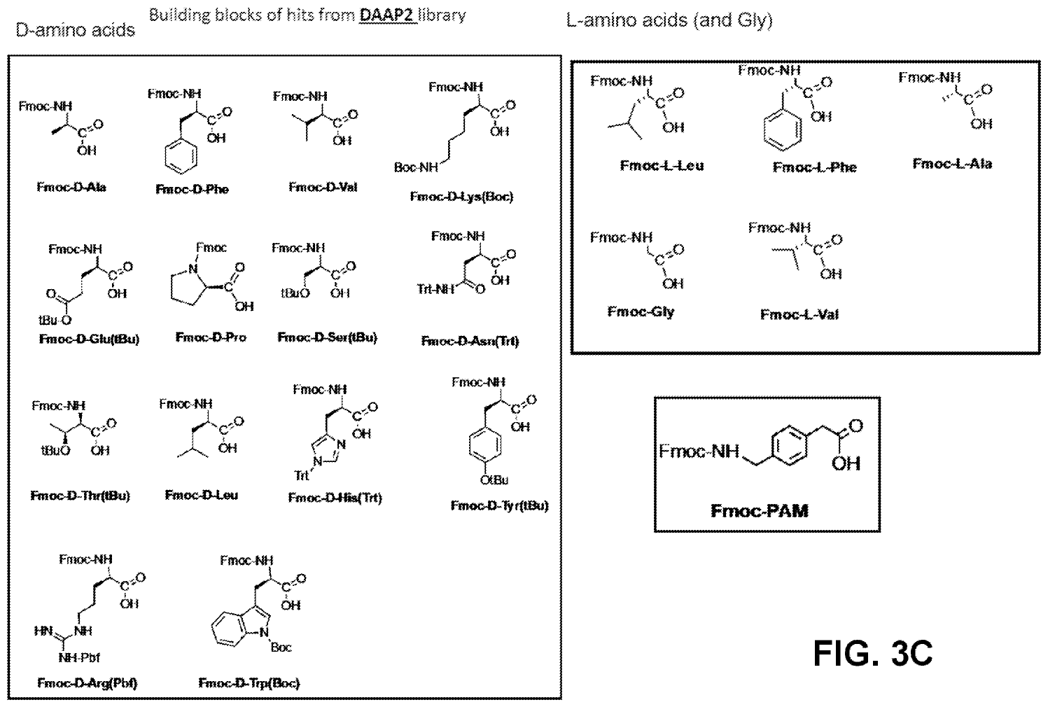

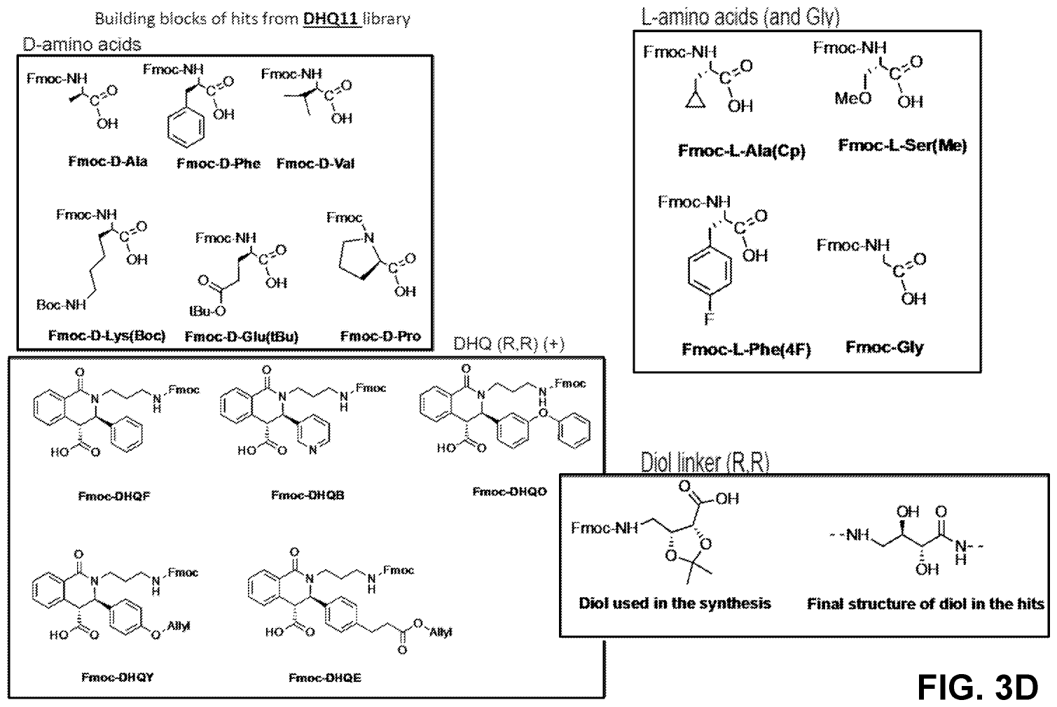

Antibody repurposing leverages the immunologic recruitment efficacy and safety of a previously FDA-approved Ab which took decades to develop and validate for clinical use, with the ability to discover and develop an affinity targeting agent to effectively repurpose the Ab for any desired target, in a matter of months. This approach enables rapid access to Ab based counter-measures without the time and cost of discovery and development, and importantly circumvents the ramp-up time for set up GMP manufacturing of the Ab on a scale suitable for the threat and exposed population level, as the Ab is already being GMP manufactured for its primary approved indication. In contrast, Technein-epitope conjugated agents are relatively easy to manufacture, and a general GMP validated process could be pre-established for rapid response. As suggested above, this approach may be extended beyond Techneins to include any target affinity molecules linked to a synthetic epitope or full antigen of an antibody. This may include small molecules, peptides, scFvs, nucleic acids, and aptamers, and Techneins. In some embodiments, the Technein is selected from those in FIGS. 2 and 3. In some embodiments, the Technein is selected from the Techneins described in PCT/US17/50119 entitled, “MASS SPECTROMETRY DISTINGUISHABLE SYNTHETIC COMPOUNDS, LIBRARIES, AND METHODS THEREOF”, PCT/US15/50306 entitled, “Affinity Reagent and Catalyst Discovery Though Fiber-Optic Array Scanning Technology”, and PCT/US21/55226 entitled, “HIGH AFFINITY NON-NATURAL LIGANDS AGAINST PROTEIN TARGETS”. The contents of which are incorporated by reference in their entirety.

### Synthetic Compounds for Repurposing of Antibodies

In one embodiment, the disclosure provides process of repurposing an antibody. In one embodiment, the process requires the non-covalent conjugation of an original antibody with a synthetic compound capable of acting as a bridge between the original antibody and the new target of the repurposed antibody. In one embodiment, the antibody and the synthetic compound are linked covalently. In one embodiment, the synthetic compound comprises the following parts:
(i) a first molecule that binds to an antibody, or to a target-binding fragment thereof; wherein the antibody originally binds to a first target; and
(ii) a second molecule that binds to a second target;
wherein the first target and the second target are different, and wherein the first molecule and the second molecule are covalently linked, directly or through a linker.

In one embodiment, the first molecule binds to the antibody variable region. In some embodiments, the first molecule comprises the first target or a fragment thereof. In some embodiments, the first molecule comprises an epitope of the antibody to be repurposed. In one embodiment, the first molecule is selected from any one of the targets of the antibodies described in Tables 1 to 3, or an antibody-binding fragment thereof. In some embodiments, the antibody is an endogenous antibody and the first molecule is selected from its natural target. In some embodiments, the first molecule is alpha-Gal. In some embodiments, the first molecule is biotin.

In some embodiments, the first molecule comprises a synthetic polymer. In some embodiments, the synthetic polymer comprises monomers that are amino acids and monomers that are not amino acids. In some embodiments, the synthetic polymers, amino acids, and monomers are selected from those in FIG. 3. In some embodiments, the synthetic polymers are also referred to as Techneins. In some embodiments, the Technein is selected from the Techneins described in PCT/US17/50119 entitled, “MASS SPECTROMETRY DISTINGUISHABLE SYNTHETIC COMPOUNDS, LIBRARIES, AND METHODS THEREOF”, PCT/US15/50306 entitled, “Affinity Reagent and Catalyst Discovery Though Fiber-Optic Array Scanning Technology”, and PCT/US21/55226 entitled, “HIGH AFFINITY NON-NATURAL LIGANDS AGAINST PROTEIN TARGETS,” the contents of all of which are incorporated by reference in their entirety. In one embodiment, the first molecule is selected from small molecules, peptides, scFvs, nucleic acids, and aptamers.

In some embodiments, the second molecule binds to the second target. In one embodiment, the second molecule comprises a synthetic polymer. In some embodiments, the synthetic polymer comprises monomers that are amino acids and monomers that are not amino acids. In some embodiments, the synthetic polymers, amino acids, and monomers are selected from those in FIG. 2. In some embodiments, the synthetic polymers are Techneins. In one embodiment, the first and/or second molecule is selected from small molecules, peptides, scFvs, nucleic acids, and aptamers.

In one embodiment, the first molecule and the second molecule are covalently linked. In one embodiment, the first molecule and the second molecule are directly linked to each other. In one embodiment, the first molecule and the second molecule are linked to each other through a linker.

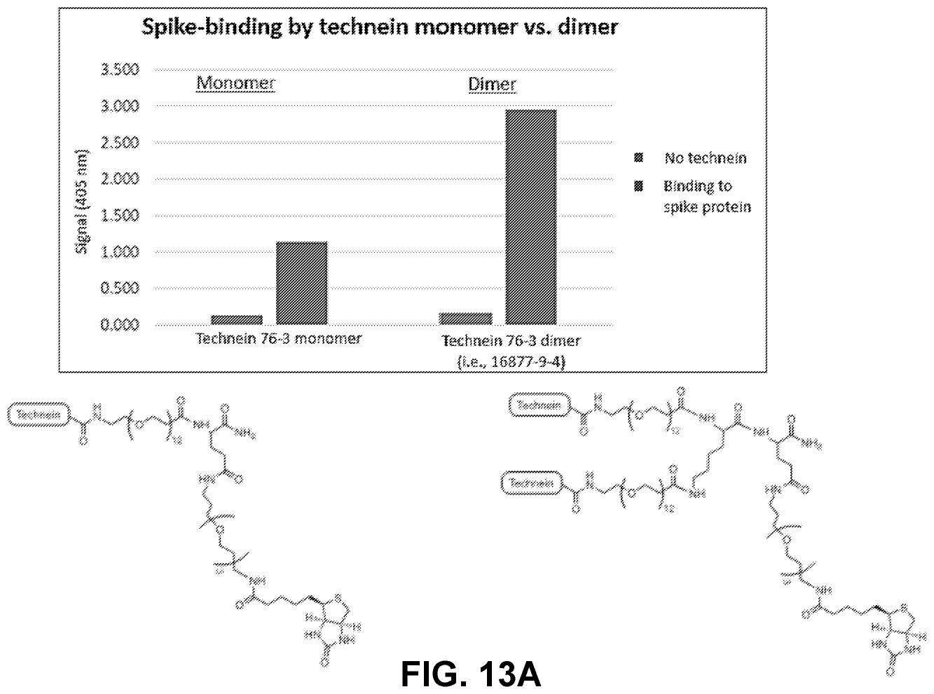

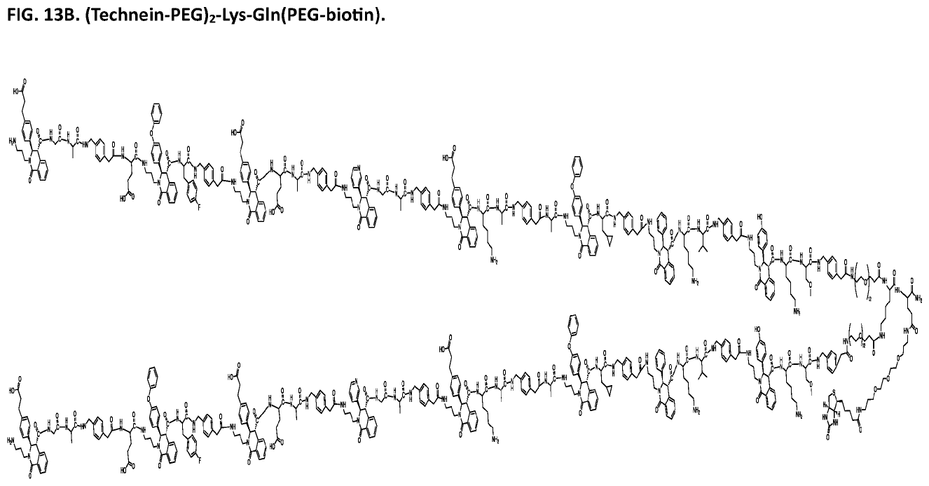

In some embodiments, the synthetic compound comprises one Technein. In some embodiments, the synthetic compound comprises two Techneins, one of which is a first molecule and the other is a second molecule. FIG. 1. In some embodiments, the synthetic compound comprises a first molecule and/or a second molecule each comprising two or more Techneins linked to each other directly or through a linker. In some embodiments, the two Techneins in the first molecule or second molecule are linked to each other as shown in FIG. 13B. In some embodiments, the first molecule comprises a Techenin dimer wherein each monomer is linked to a PEG molecule and wherein the two PEG molecules converge on a Lys-Gln linker, which may be connected to a PEG, which may be linked to a second molecule.

In some embodiments, the linker comprises Polyethylene glycol (PEG), PEG. In some embodiments, the linker comprises PEG, peptides, alkyl chains, cysteines, lysines, glutamines, maleimides, dibenzyclooctanes, or combinations thereof.

### Antibodies

Any antibody may be repurposed using the methods of the disclosure. In some embodiments, the antibody has been previously approved by the Food and Drug Administration (FDA), European Medicines Agency (EMA), SwissMedic, China Food and Drug Administration (CFDA), Japanese Pharmaceuticals and Medical Devices Agency (PMDA), or any other health agency responsible for antibody approval. In some embodiments, the antibody is an endogenous antibody (endogenous to the subject to be treated with the repurposed antibody). In some embodiments, the endogenous antibody is expressed recombinantly and combined with the synthetic compound in vitro.

Examples of antibodies that have been previously approved may be found on health agencies' websites. In some embodiments, the antibody is selected from those in Table 1.

TABLE 1
Exemplary Antibodies for Repurposing Purposes

| International | | | |
|---|---|---|---|
| Non-Proprietary | | Original | |
| Name | Brand Name | Target | Indication First Approved or Reviewed |
| Zimberelimab | | PD-1 | Classical Hodgkin's lymphoma |
| Vedolizumab | Entyvio | α4β7 integrin | Ulcerative colitis, Crohn disease |
| Ustekinumab | Stelara | IL-12/23 | Psoriasis |
| Ublituximab | BRIUMVI | CD20 | Multiple sclerosis |
| Tremelimumab | Imjudo | CTLA-4 | Antineoplastic; liver cancer |
| Trastuzumab | (Pending) | HER2 | Breast cancer |
| duocarmazine | | | |
| Trastuzumab | Herceptin | HER2 | Breast cancer |
| Tralokinumab | Adtralza | IL-13 | Atopic dermatitis |
| Tositumomab- | Bexxar | CD20 | Non-Hodgkin Lymphoma |
| l131 | | | |
| Toripalimab | Tuoyi | PD-1 | Nasopharyngeal carcinoma |
| Tocilizumab | RoActemra, | IL-6R | Rheumatoid arthritis |
| | Actemra | | |
| Tixagevimab, | Evusheld | SARS-CoV-2 | COVID-19 |
| cilgavimab | | | |
| Tisotumab | TIVDAK | Tissue factor | Cervical cancer |
| vedotin, | | | |
| tisotumab | | | |
| vedotin-tftv | | | |
| Tislelizumab | (Pending) | PD-1 | Esophageal squamous cell carcinoma |
| Tildrakizumab | Ilumya | IL-23 p19 | Plaque psoriasis |
| (tildrakizumab- | | | |
| asmn) | | | |
| Tezepelumab | Tezspire | Thymic | Severe asthma |
| | | stromal | |
| | | lymphopoietin | |
| Teprotumumab | Tepezza | IGF-1R | Thyroid eye disease |
| (teprotumumab- | | | |
| trbw) | | | |
| Teplizumab | TZIELD | CD3 | Type 1 diabetes |
| Teclistamab | TECVAYLI | BCMA, CD3 | Multiple myeloma |
| Tebentafusp | KIMMTRAK | gp100, CD3 | Metastatic uveal melanoma |
| Talquetamab | (Pending) | G protein- | Multiple myeloma |
| | | coupled | |
| | | receptor 5D, | |
| | | CD3 | |
| Tagitanlimab | (Pending) | PD-L1 | Nasopharyngeal carcinoma, solid tumor |
| Tafolecimab | (Pending) | PCSK9 | Primary hypercholesterolemia (including |
| | | | heterozygous familial hypercholesterolemia |
| | | | and non-familial hypercholesterolemia) and |
| | | | mixed dyslipidemia |
| Tafasitamab | Monjuvi, | CD19 | Diffuse large B-cell lymphoma |
| (tafasitamab-cxix) | Minjuvi | | |
| Sutimlimab, | Enjaymo | C1s | Cold agglutinin disease |
| sutimlimab-jome | | | |
| Sugemalimab | Cejemly ® | PD-L1 | Non-small cell lung cancer |
| Spesolimab | SPEVIGO ® | IL-36 receptor | Generalized pustular psoriasis |
| Sotrovimab | Xevudy | SARS-CoV-2 | COVID-19 |
| Socazolimab | (Pending) | PD-L1 | Cervical cancer |
| Sintilimab | Tyvyt | PD-1 | Non-small cell lung cancer |
| Siltuximab | Sylvant | IL-6 | Castleman disease |
| Serplulimab | HANSIZHUANG | PD-1 | Microsatellite instability-high solid tumors |
| Secukinumab | Cosentyx | IL-17a | Psoriasis |
| Satralizumab | Enspryng | IL-6R | Neuromyelitis optica spectrum disorder |
| (satralizumab- | | | |
| mwge) | | | |
| Sarilumab | Kevzara | IL-6R | Rheumatoid arthritis |
| Sacituzumab | TRODELVY | TROP-2 | Triple-neg. breast cancer |
| govitecan | | | |
| (sacituzumab | | | |
| govitecan-hziy) | | | |
| Rozanolixizumab | (Pending) | FcRn | Generalized myasthenia gravis |
| Romosozumab | Evenity | Sclerostin | Osteoporosis in postmenopausal women at |
| (romosozumab- | | | increased risk of fracture |
| aqqg) | | | |
| Rituximab | MabThera, | CD20 | Non-Hodgkin lymphoma |
| | Rituxan | | |
| Risankizumab | Skyrizi | IL-23 p19 | Plaque psoriasis |
| (risankizumab- | | | |
| rzaa) | | | |
| Ripertamab | Anpingxi | CD20 | Non-Hodgkin's lymphoma |
| Retifanlimab | Zynyz | PD-1 | Merkel cell carcinoma |
| Reslizumab | Cinqaero, | IL-5 | Asthma |
| | Cinqair | | |
| Relatlimab | Opdualag | LAG-3 | Melanoma |
| | (relatlimab + | | |
| | nivolumab | | |
| | combo) | | |
| Regdanvimab | Regkirona | SARS-CoV-2 | COVID-19 |
| Raxibacumab | (Pending) | B. anthrasis | Anthrax infection |
| | | PA | |
| Ravulizumab | Ultomiris | C5 | Paroxysmal nocturnal hemoglobinuria |
| (ravulizumab- | | | |
| cwvz) | | | |
| Ranibizumab | Lucentis | VEGF | Macular degeneration |
| Ramucirumab | Cyramza | VEGFR2 | Gastric cancer |
| Racotumomab | Vaxira ® | GM3 | Non-small cell lung cancer |
| Pucotenlimab | Puyouheng | PD-1 | Metastatic microsatellite instability-high |
| | | | (MSI-H) or mismatch repair deficient (dMMR) |
| | | | advanced solid tumors, |
| Prolgolimab | Forteca | PD-1 | Melanoma |
| Polatuzumab | Polivy | CD79b | Diffuse large B-cell lymphoma |
| vedotin | | | |
| (polatuzumab | | | |
| vedotin-piiq) | | | |
| Pertuzumab | Perjeta | HER2 | Breast Cancer |
| Penpulimab | ANNIKO | PD-1 | Metastatic nasopharyngeal carcinoma |
| Pembrolizumab | Keytruda | PD1 | Melanoma |
| Panitumumab | Vectibix | EGFR | Colorectal cancer |
| Palivizumab | Synagis | RSV | Prevention of respiratory syncytial virus |
| | | | infection |
| Pabinafusp alfa | IZCARGO | Transferrin | Mucopolysaccharidosis type II |
| | | receptor | |
| Ozoralizumab | Nanozora ® | TNF, albumin | Rheumatoid arthritis |
| Ormutivimab | (Pending) | Rabies virus | Post-exposure prophylaxis of rabies |
| | | surface | |
| | | glycoprotein 4 | |
| Omburtamab | (Pending) | B7-H3 | Central nervous system/leptomeningeal |
| | | (CD276) | metastasis from neuroblastoma |
| Omalizumab | Xolair | IgE | Asthma |
| Olokizumab | ARTLEGIA | IL-6 | Rheumatoid arthritis |
| Olaratumab | Lartruvo | PDGFRα | Soft tissue sarcoma |
| Ofatumumab | Arzerra | CD20 | Chronic lymphocytic leukemia |
| Ocrelizumab | OCREVUS | CD20 | Multiple sclerosis |
| Obinutuzumab | Gazyva, | CD20 | Chronic lymphocytic leukemia |
| | Gazyvaro | | |
| Obiltoxaximab | Anthim | B. anthrasis | Prevention of inhalational anthrax |
| | | PA | |
| Nivolumab | Opdivo | PD1 | Melanoma, non-small cell lung cancer |
| Nirsevimab | Beyfortus | RSV | Respiratory syncytial virus infection |
| Nimotuzumab | TheraCIM, | EGFR | Head and neck cancer |
| | BIOMAB-EGFR | | |
| Netakimab | Efleira | IL-17 | Plaque psoriasis |
| Nemolizumab | Mitchga | IL-31 receptor | Pruritus with atopic dermatitis |
| | | alpha | |
| Necitumumab | Portrazza | EGFR | Non-small cell lung cancer |
| Nebacumab | Centoxin | Endotoxin | Gram-negative sepsis |
| Naxitamab-gqgk | DANYELZA | GD2 | High-risk neuroblastoma and refractory |
| | | | osteomedullary disease |
| Natalizumab | Tysabri | a4 integrin | Multiple sclerosis |
| Narsoplimab | (Pending) | MASP-2 | Hematopoietic stem cell transplant- |
| | | | associated thrombotic microangiopathies |
| Narlumosbart | (Pending) | RANK-L | Giant cell tumor of bone |
| Muromonab-CD3 | Orthoclone | CD3 | Reversal of kidney transplant rejection |
| | Okt3 | | |
| Moxetumomab | Lumoxiti | CD22 | Hairy cell leukemia |
| pasudotox | | | |
| (moxetumomab | | | |
| pasudotox-tdfk) | | | |
| Mosunetuzumab | Lunsumio | CD20, CD3 | Follicular lymphoma |
| Mogamulizumab | Poteligeo | CCR4 | Mycosis fungoides or Sézary syndrome |
| (mogamulizumab- | | | |
| kpkc) | | | |
| Mirvetuximab | ELAHERE | Folate | Ovarian cancer |
| soravtansine | | receptor | |
| | | alpha | |
| Mirikizumab | Omvoh | IL-23p19 | Ulcerative colitis |
| Mepolizumab | Nucala | IL-5 | Severe eosinophilic asthma |
| Margetuximab- | MARGENZA | HER2 | HER2+ metastatic breast cancer |
| cmkb | | | |
| Loncastuximab | Zynlonta | CD19 | Diffuse large B-cell lymphoma |
| tesirine | | | |
| Levilimab | Ilsira | IL-6 receptor | Inflammation due to COVID-19 infection |
| Lecanemab | Leqembi | Amyloid beta | Alzheimer's disease |
| | | protofibrils | |
| Lebrikizumab | (Pending) | IL-13 | Atopic dermatitis |
| Lanadelumab | Takhzyro | Plasma | Hereditary angioedema attacks |
| (lanadelumab- | | kallikrelin | |
| flyo) | | | |
| Ixekizumab | Taltz | IL-17a | Psoriasis |
| Itolizumab | Alzumab | CD6 | Psoriasis |
| Isatuximab | Sarclisa | CD38 | Multiple myeloma |
| (isatuximab-irfc) | | | |
| Ipilimumab | Yervoy | CTLA-4 | Metastatic melanoma |
| Inotuzumab | BESPONSA | CD22 | Acute lymphoblastic leukemia |
| ozogamicin | | | |
| Infliximab | Remicade | TNF | Crohn disease |
| Inetetamab | Cipterbin | HER2 | HER2-positive metastatic breast cancer |
| Inebilizumab | Uplizna | CD19 | Neuromyelitis optica spectrum disorders |
| (inebilizumab- | | | |
| cdon) | | | |
| Idarucizumab | Praxbind | Dabigatran | Reversal of dabigatran-induced |
| | | | anticoagulation |
| Ibritumomab | Zevalin | CD20 | Non-Hodgkin lymphoma |
| tiuxetan | | | |
| Ibalizumab | Trogarzo | CD4 | HIV infection |
| (ibalizumab-uiyk) | | | |
| Guselkumab | TREMFYA | IL-23 p19 | Plaque psoriasis |
| Golimumab | Simponi | TNF | Rheumatoid and psoriatic arthritis, ankylosing |
| | | | spondylitis |
| Glofitamab | COLUMVI ® | CD20, CD3e | Diffuse large B-cell lymphoma |
| Geptanolimab | (Pending) | PD-1 | Peripheral T cell lymphoma |
| (Genolimzumab) | | | |
| Gemtuzumab | Mylotarg | CD33 | Acute myeloid leukemia |
| ozogamicin | | | |
| Galcanezumab | Emgality | CGRP | Migraine prevention |
| (galcanezumab- | | | |
| gnlm) | | | |
| Fremanezumab | Ajovy | CGRP | Migraine prevention |
| (fremanezumab- | | | |
| vfrm) | | | |
| Faricimab, | Vabysmo | VEGF-A, Ang-2 | Neovascular age-related macular |
| faricimab-svoa | | | degeneration, diabetic macular edema |
| Evolocumab | Repatha | PCSK9 | High cholesterol |
| Evinacumab | Evkeeza | Angiopoietin- | Homozygous familial hypercholesterolemia |
| | | like 3 | |
| Erenumab | Aimovig | CGRP | Migraine prevention |
| (erenumab-aooe) | | receptor | |
| Eptinezumab | VYEPTI | CGRP | Migraine prevention |
| (eptinezumab- | | | |
| jjmr) | | | |
| Epcoritamab | (Pending) | CD20, CD3 | Diffuse large B-cell lymphoma |
| Envafolimab | ENWEIDA | PD-L1 | Microsatellite instability-high or deficient |
| | | | MisMatch Repair advanced solid tumors |
| Enfortumab | Padcev | Nectin-4 | Urothelial cancer |
| vedotin | | | |
| (enfortumab | | | |
| vedotin-ejfv) | | | |
| Emicizumab | Hemlibra | Factor Ixa, X | Hemophilia A |
| Emapalumab | Gamifant | IFNg | Primary hemophagocytic lymphohistiocytosis |
| (emapalumab- | | | |
| lzsg) | | | |
| Elranatamab | (Pending) | BCMA, CD3 | Multiple myeloma |
| Elotuzumab | Empliciti | SLAMF7 | Multiple myeloma |
| Efalizumab | Raptiva | CD11a | Psoriasis |
| Edrecolomab | Panorex | EpCAM | Colon cancer |
| Eculizumab | Soliris | C5 | Paroxysmal nocturnal hemoglobinuria |
| Durvalumab | IMFINZI | PD-L1 | Bladder cancer |
| Dupilumab | Dupixent | IL-4R α | Atopic dermatitis |
| Dostarlimab | Jemerli | PD-1 | Endometrial cancer |
| Donanemab | (Pending) | Amyloid beta, | Alzheimer's disease |
| | | N3pG (N- | |
| | | terminal | |
| | | truncated) | |
| Docaravimab and | Twinrab | Rabies virus | Rabies exposure |
| Miromavimab | | | |
| Disitamab | Aidixi | HER2 | Gastric cancer |
| vedotin | | | |
| Dinutuximab | Unituxin | GD2 | Neuroblastoma |
| Denosumab | Prolia | RANK-L | Bone Loss |
| Daratumumab | Darzalex | CD38 | Multiple myeloma |
| Daclizumab | Zinbryta; | CD25 | Multiple sclerosis#; prevention of kidney |
| | Zenapax | | transplant rejection# |
| Crovalimab | (Pending) | Complement | Atypical hemolytic uremic syndrome |
| | | C5 | |
| Crizanlizumab | Adakveo | CD62 (aka P- | Sickle cell disease |
| (crizanlizumab- | | selectin) | |
| tmca) | | | |
| Cosibelimab | (Pending) | PD-L1 | Squamous cell carcinoma |
| Concizumab | (Pending) | Tissue factor | Hemophilia A or B |
| | | pathway | |
| | | inhibitor | |
| Cetuximab | Akalux ® IV | EGFR | Head and neck cancer |
| saratolacan | Infusion | | |
| Cetuximab | Erbitux | EGFR | Colorectal cancer |
| Certolizumab | Cimzia | TNF | Crohn disease |
| pegol | | | |
| Cemiplimab | Libtayo | PD-1 | Cutaneous squamous cell carcinoma |
| (cemiplimab-rwlc) | | | |
| Catumaxomab | Removab | EPCAM, CD3 | Malignant ascites |
| Casirivimab + | REGEN-COV, | SARS-CoV-2 | COVID-19 |
| imdevimab | Ronapreve | | |
| Caplacizumab | Cablivi | von | Acquired thrombotic thrombocytopenic |
| (caplacizumab- | | Willebrand | purpura |
| yhdp) | | factor | |
| Canakinumab | Ilaris | IL-1β | Muckle-Wells syndrome |
| Camrelizumab | AiRuiKa | PD-1 | Hodgkin's lymphoma |
| Cadonilimab | [symbol] ® | PD-1, CTLA4 | Cervical cancer |
| Burosumab | Crysvita | FGF23 | X-linked hypophosphatemia |
| (burosumab- | | | |
| twza) | | | |
| Brolucizumab | Beovu | VEGF-A | Neovascular age- |
| (brolucizumab- | | | related macular degeneration |
| dbll) | | | |
| Brodalumab | Siliq, LUMICEF, | IL-17R | Plaque psoriasis |
| | Kyntheum | | |
| Brentuximab | Adcetris | CD30 | Hodgkin lymphoma, systemic anaplastic large |
| vedotin | | | cell lymphoma |
| Blinatumomab | Blincyto | CD19, CD3 | Acute lymphoblastic leukemia |
| Bimekizumab | Bimzelx | IL-17A and IL- | Psoriasis |
| | | 17F | |
| | | (overlapping | |
| | | binding site) | |
| Bezlotoxumab | Zinplava | Clostridium | Prevention of Clostridium difficile infection |
| | | difficile | recurrence |
| | | enterotoxin B | |
| Bevacizumab | Avastin | VEGF | Colorectal cancer |
| Benralizumab | Fasenra | IL-5R α | Asthma |
| Belimumab | Benlysta | BLyS | Systemic lupus erythematosus |
| Belantamab | BLENREP | B-cell | Multiple myeloma |
| mafodotin | | maturation | |
| (belantamab | | antigen | |
| mafodotin-blmf) | | | |
| Basiliximab | Simulect | IL-2R | Prevention of kidney transplant rejection |
| Avelumab | Bavencio | PD-L1 | Merkel cell carcinoma |
| Atoltivimab, | Inmazeb | Ebola virus | Ebola virus infection |
| maftivimab, and | | | |
| odesivimab-ebgn | | | |
| Atezolizumab | Tecentriq | PD-L1 | Bladder cancer |
| Ansuvimab-zykl | Ebanga | Ebola virus | Ebola virus infection |
| | | glycoprotein | |
| Anifrolumab, | Saphnelo | IFNAR1 | Systemic lupus erythematosus |
| anifrolumab-fnia | | | |
| Amubarvimab + | | SARS-CoV-2 | SARS-CoV-2 infection |
| Romlusevimab | | | |
| Amivantamab | RYBREVANT | EGFR, cMET | NSCLC w/EGFR exon 20 insertion mutations |
| Alirocumab | Praluent | PCSK9 | High cholesterol |
| Alemtuzumab | Lemtrada; | CD52 | Multiple sclerosis; chronic myeloid leukemia# |
| | MabCampath, | | |
| | Campath-1H | | |
| Aducanumab | ADUHELM | Amyloid beta | Alzheimer's disease |
| Ado-trastuzumab | Kadcyla | HER2 | Breast cancer |
| emtansine | | | |
| Adebrelimab | (Pending) | PD-L1 | Small cell lung cancer |
| Adalimumab | Humira | TNF | Rheumatoid arthritis |
| Abciximab | Reopro | GPIIb/IIIa | Prevention of blood clots in angioplasty |
| [fam-]trastuzumab | Enhertu | HER2 | HER2+ metastatic breast cancer |
| deruxtecan, (fam- | | | |
| trastuzumab | | | |
| deruxtecan-nxki) | | | |
| | RabiShield | Rabies virus G | Rabies exposure |
| | | glycoprotein | |
| Pozelimab | (Pending) | Complement | CHAPLE disease |
| | | 5 | |
| Enlonstobart | Enshuxing | PD-1 | Cervical cancer |

In some embodiments, the antibody is selected from Trastuzumab (Herceptin), Adalimumab (Humira), Bevacizumab (Avastin), Rituximab (Rituxan/MabThera), Infliximab and (Remicade). In some embodiments, the antibody is Herceptin. In some embodiments, the antibody is selected from an endogenous anti-alpha-Galactosidase (Gal) antibody. Alpha-Gal may be also referred to as α-Gal and α-Gal herein and in the art.

Chimeric antigen receptor T-cell (CAR-T) therapies also rely on antibody-like molecules for their mechanism of action. Accordingly, antibody repurposing may also be used to repurpose CAR-T cells by altering their target in a similar manner. Examples of CAR-T cell treatments that may be repurposed include, but are not limited to, those in Table 2.

TABLE 2
Exemplary FDA-Approved CAR T-cell therapies

| | Brand | Target | | |
|---|---|---|---|---|
| Generic Name | Name | Antigen | Targeted Disease | Patient Population |
| Tisagenlecleucel | Kymriah | CD19 | B-cell acute | Children and young adults |
| | | | lymphoblastic | with refractory or relapsed |
| | | | leukemia (ALL) | B-cell ALL |
| | | | B-cell non-Hodgkin | Adults with relapsed or |
| | | | lymphoma (NHL) | refractory B-cell NHL |
| Axicabtagene ciloleucel | Yescarta | CD19 | B-cell non-Hodgkin | Adults with relapsed or |
| | | | lymphoma (NHL) | refractory B-cell NHL |
| | | | Follicular lymphoma | Adults with relapsed or |
| | | | | refractory follicular |
| | | | | lymphoma |
| Brexucabtagene | Tecartus | CD19 | Mantle cell | Adults with relapsed or |
| autoleucel | | | lymphoma (MCL) | refractory MCL |
| | | | B-cell acute | Adults with refractory or |
| | | | lymphoblastic | relapsed B-cell ALL |
| | | | leukemia (ALL) | |
| Lisocabtagene maraleucel | Breyanzi | CD19 | B-cell non-Hodgkin | Adults with relapsed or |
| | | | lymphoma (NHL) | refractory B-cell NHL |
| Idecabtagene vicleucel | Abecma | BCMA | Multiple myeloma | Adults with relapsed or |
| | | | | refractory multiple |
| | | | | myeloma |

In some embodiments, the CAR-T cells originally target CD19, CD20, or BCMA.

TABLE 3
Non-comprehensive list of ongoing CAR-T cell therapy studies in solid organ malignancies

| | | Recruiting | | |
|---|---|---|---|---|
| Cancer type | NCT number | status | Brief title | Cell target |
| Brain Cancers | NCT02208362 | Ongoing Phase | Genetically Modified T-cells in | IL13Ra2 |
| (Glioblastoma) | | I | Treating Patients with Recurrent or | |
| | | | Refractory Malignant Glioma | |
| | NCT03726515 | Ongoing Phase | CART-EGFRvIII + Pembrolizumab in | EGFRvIII |
| | | I | GBM | |
| | NCT01454596 | Completed | CAR T Cell Receptor | EGFRvIII |
| | | recruiting/ | Immunotherapy Targeting EFGRvIII | |
| | | phase I | for Patients with Malignant Gliomas | |
| | | | Expressing EGFRvIII | |
| | NCT01109095 | Completed | CMV-specific Cytotoxic T | HER2 |
| | | recruiting/ | Lymphocytes Expressing CAR | |
| | | phase I | Targeting HER2 in Patients with | |
| | | | GBM | |
| Gastrointestinal | NCT01373047 | Completed | CEA-Expressing Liver Metastases | CEA |
| Cancers | | recruiting/ | Safety Study of Intrahepatic | |
| | | phase I | Infusions of Anti-CEA Designer T | |
| | | | Cells | |
| | NCT03682744 | Ongoing phase | CAR-T Intraperitoneal Infusions for | CEA |
| | | I | CEA-Expressing Adenocarcinoma | |
| | | | Peritoneal Metastases or Malignant | |
| | | | Ascites (IPC) | |
| | NCT01897415 | Completed | Autologous Redirected RNA Meso | Mesothelin |
| | | recruiting/ | CAR T Cells for Pancreatic Cancer | |
| | | phase | | |
| | NCT03323944 | Ongoing phase | CAR T Cell Immunotherapy for | Mesothelin |
| | | I | Pancreatic Cancer | |
| | NCT03159819 | Ongoing phase | Clinical Study of CAR-CLD18 T Cells | Claudin |
| | | I | in Patients with Advanced Gastric | 18.2 |
| | | | Adenocarcinoma and Pancreatic | |
| | | | Adenocarcinoma | |
| | NCT02744287 | Ongoing phase | Safety and Activity Study of PSCA- | PSCA |
| | | I | Targeted CAR-T Cells (BPX-601) in | |
| | | | Subjects with Selected Advanced | |
| | | | Solid Tumors | |
| Renal Cancer | N/A | Preclinical | | Carboxy- |
| | | | | anhydrase |
| | | | | IX (CAIX) |
| | NCT03393936 | Ongoing phase | Safety and Efficacy of CCT301 CAR-T | AXL |
| | | I | in Adult Subjects with Recurrent or | |
| | | | Refractory Stage IV Renal Cell | |
| | | | Carcinoma | |
| Prostate Cancer | NCT03089203 | Ongoing phase | CART-PSMA-TGFβRDN Cells for | PSMA |
| | | I | Castrate-Resistant Prostate Cancer | |
| | NCT03873805 | Ongoing phase | PSCA-CAR T Cells in Treating | PSCA |
| | | I | Patients With PSCA+ Metastatic | |
| | | | Castration Resistant Prostate Cancer | |
| Ovarian Cancer | NCT03585764 | Ongoing phase | MOv19-BBz CAR T Cells in aFR | Folate |
| | | I | Expressing Recurrent High Grade | receptor- |
| | | | Serous Ovarian, Fallopian Tube, or | alpha |
| | | | Primary Peritoneal | |
| | NCT02498912 | Ongoing phase | Cyclophosphamide Followed by | MUC16 |
| | | I | Intravenous and Intraperitoneal | |
| | | | Infusion of Autologous T Cells | |
| | | | Genetically Engineered to Secrete | |
| | | | IL-12 and to Target the MUC16ecto | |
| | | | Antigen in Patients with Recurrent | |
| | | | MUC16ecto+ Solid Tumors | |
| | NCT02792114 | Ongoing phase | T-Cell Therapy for Advanced Breast | Mesothelin |
| | | I | Cancer | |
| | NCT02442297 | Ongoing phase | T Cell Expressing HER2-specific | HER2 |
| | | I | Chimeric Antigen Receptors (CAR) | |
| | | | for Patients with HER2-Positive CNS | |
| | | | Tumors | |
| | NCT03696030 | Ongoing phase | HER2-CAR T Cells in Treating | HER2 |
| | | I | Patients with Recurrent Brain or | |
| | | | Leptomeningeal Metastases | |
| | NCT04020575 | Ongoing phase | Autologous huMNC2-CAR44 T Cells | MUC1 |
| | | I | for Breast Cancer Targeting Cleaved | |
| | | | Form of MUC1 | |
| Thoracic Cancer | NCT02414269 | Ongoing phase | Malignant Pleural Disease Treated | Mesothelin |
| | | I | with Autologous T Cells Genetically | |
| | | | Engineered to Target the Cancer- | |
| | | | Cell Surface Antigen Mesothelin | |
| | NCT0305429 | Ongoing phase | CAR T Cells in Mesothelin | Mesothelin |
| | | I | Expressing Cancers | |
| | NCT02706392 | Ongoing phase | Genetically Modified T-Cell Therapy | ROR1 |
| | | I | in Treating Patients with Advanced | |
| | | | ROR1+ Malignancies | |

### Targets

The first/original target and the second/new target may be any target. The first target determines the antibody, and it is generally irrelevant what the target is because the antibody's ability to recognize it and bind it will be eliminated by the binding of the synthetic compound to its variable region. The first or original targets of some of the antibodies that may be used according to this disclosure are listed, but are not limited to, in Tables 1-3. The antibody may be recognized by any first molecule. Examples of first molecules are described above. When the first molecule is a Technein, a Technein must be obtained that binds to the antibody. Once a second target is chosen, the next step is to obtain a Technein that specifically binds to it. In one embodiment, the Technein is identified by screening a library of Techneins. For some antibodies, the first molecule component of the synthetic compound of the disclosure does not need to be a Technein but may be the antibody's natural target/ligand, or a fragment thereof (e.g., an epitope). In one embodiment, if the antibody to be repurposed is an anti-biotin antibody, the first molecule of the synthetic compound may comprise biotin. If the antibody is an anti-alpha-Gal antibody, the first molecule may comprise alpha-Gal.

The second target dictates the new purpose for the repurposed antibody. A second molecule must be identified that binds to the second target and may be linked to the first molecule, directly or through a linker. In some embodiments, the second molecule is a Technein. In some embodiments, the Technein is identified by screening a library of Techneins for those that bind to the second target.

The targets may be any type of biologic molecule including, for example, simple intermediary metabolites, sugars, lipids, and hormones as well as macromolecules such as complex carbohydrates, phospholipids, nucleic acids, and proteins. Some exemplary categories of targets include, but are not limited to, viral antigens, bacterial antigens, fungal antigens, tick antigens, protozoa and other parasitic antigens, tumor antigens, antigens involved in autoimmune disease, allergy and graft rejection, and other miscellaneous antigens. In one embodiment, the target is selected from microbial antigens, such as viral, fungal, or bacterial; or therapeutic antigens such as antigens associated with cancerous cells or growths, or autoimmune disorders. In some embodiments, the target is selected from a small molecule, nucleotide, polynucleotide, peptide, polypeptide, protein, lipid, carbohydrate, other immunogenic molecules, and a combination thereof. Exemplary diseases, pathogens, pathogen polypeptides, and disease-associated polypeptides are known and additional targets may be easily identified by one of ordinary skill in the art. First targets and second targets may be selected from the same pool of molecules. Thus, any reference to a second target is also an example of a first target. The first target and the second target of the synthetic compounds of the disclosure must be different in order for the antibody to be repurposed.

In some embodiments, the repurposed antibodies may be used as immunotherapeutics for vaccination against pathogens, or for the treatment of resulting infections, including persistent viral infections. The pathogens may include bacteria, protozoans, viruses, and prions, and other prion-like particles that cause diseases and disorders. Bacterial pathogens include, for example, species of Escherichia, Klebsiella, Staphylococcus, Acinetobacter, and Pseudomonas, particularly drug-resistant species and strains. Among the bacteria, are strains/species of Salmonella, such as S. typhimurium. Among the virus are retroviridae (e.g., HIV, including a HIV Fusion Peptide antigen), Orthomyxoviridae, Paramyxoviridae, Arenaviridae, 5 Filoviridae, and/or Coronaviridae (e.g., a SARS-COV, SARS-COV-2 Fusion Peptide, and/or PEDV).

Non-limiting examples of viral antigens that may be chosen as second targets include, antigens from Coronaviridae viruses (Severe Acute Respiratory Syndrome coronavirus (SARS-CoV), Middle East respiratory syndrome-related coronavirus (MERS-COV), and the Severe Acute Respiratory Syndrome Coronavirus 2 (SARS-COV-2), retroviral antigens such as retroviral antigens the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, for example, hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpl, gpll, and other varicella zoster viral antigen components; Japanese encephalitis viral antigens such as proteins E, M-E, M-E-NS 1, NS 1, NS 1-NS2A, 80% E, and other Japanese encephalitis viral antigen components; rabies viral antigens such as rabies glycoprotein, rabies nucleoprotein and other rabies viral antigen components. See Fundamental Virology, Second Edition, e's. Fields, B. N. and Knipe, D. M. (Raven Press, New York, 1991) for additional examples of viral antigens.

In some embodiments, the target is a viral protein from the Severe Acute Respiratory Syndrome coronavirus (SARS-COV), Middle East respiratory syndrome-related coronavirus (MERS-COV), or the Severe Acute Respiratory Syndrome Coronavirus 2 (SARS-COV-2). These three viruses mediate viral invasion primarily through the binding of spike protein (S protein) to host cell receptors and determine viral tissues or host tropism. The host cell receptor protein for SARS-COV-2 is angiotensin-converting enzyme 2 (ACE2). The spike protein (S protein) binds to the ACE2 receptor and is cleaved by the host protease into the S1 polypeptide containing the Receptor binding domain (SARS-COV-2 RBD) and the S2 polypeptide responsible for mediating the fusion of the virus with the cell membrane and thus invading the host. In one embodiment of the disclosure, the coronavirus SARS-COV-2 spike protein (SARS-COV-2 S protein), its extracellular region, the S1 subunit or the receptor binding region; are used as targets. In one embodiment, the target protein is from the virus envelope (E), membrane (M), or nucleocapsid (N). In some embodiments, the coronavirus is a variant of SARS-COV-2 selected from the group consisting of the Alpha, Beta, Gamma, Delta, and Epsilon variants.

In some embodiments, the virus is an orthomyxoviridae virus, such as Influenza virus A, B and C infections rely primarily on two envelope proteoglycans: haemagglutinin (HA) and neuraminidase (NA), which are responsible for viral attachment and cellular invasion into viral particles. Influenza virus infection is triggered by attachment of the haemagglutinin (HA) protein to sialic acid-containing cell receptors (glycoproteins and glycolipids) on the surface of the virion. The neuraminidase (NA) protein mediates the processing of the sialic acid receptor and viral invasion of cells is dependent on HA-dependent receptor-mediated cytokinesis. In one embodiment of the disclosure, the influenza A H5N1 haemagglutinin (HA) protein is used as an antigen, and the influenza B haemagglutinin protein (HA1 subunit) can also be used as a target.

In some embodiments, the virus is a Filoviridae virus, which may be represented by the ebolaviruses of the genus Ebolavirus and the marburgviruses of the genus Marburgvirus. The only protein present on the surface of Ebola viruses is the glycoprotein (GP). The trimer of GP1,2 which forms the surface spike of the virus and is composed of two subunits, GP1 and GP2, linked by disulfide bonds. GP1 is known to mediate viral attachment to host cells and GP2 is involved in membrane fusion. In one embodiment, the Ebola virus glycoprotein (GP) is selected as a target, such as the GP extracellular structural domain, subunit GP proteins (GP1 and/or GP2).

In some embodiments, the virus is a Flaviviridae virus, whose family of viruses mainly includes the genera Flavivirus, Pestivirus, Pegivirus and Hepacivirus, wherein the Flaviviridae include Zika virus (ZIKV), Dengue fever (DV), West Nile virus, Japanese encephalitis virus and yellow fever virus. The Hepacivirus includes hepatitis C virus (HCV). The flavivirus envelope protein plays an important role in host cell virus infection, mediating the entry of the virus into the host cell. It consists of three separate structural envelope domains I, II and III (EDI, EDII and EDIII). EDI is a structural central domain of the envelope protein that stabilizes the overall orientation of the protein, and glycosylation sites in EDI are associated with viral production, pH sensitivity and neuroinvasiveness. EDII plays an important role in membrane fusion due to the immunological advantages of fusion loop epitopes and envelope dimeric epitopes. In addition, EDIII is a major target for neutralizing antibodies. The Zika virus envelope protein (“E” or “EP”) consists of three distinct structural domains. E structural domain I (E-DI) is the central structural domain that organizes the entire E protein structure. E structural domain II (E-DII) is formed by two extended loops that protrude from E-DI and are located in pockets at E-DI and E structural domain III (E-DIII). E-DIII is an immunoglobulin-like structural domain, which forms small protrusions on the surface of otherwise smooth, spherical, mature viral particles and is thought to interact with cellular receptors on target cells. In one embodiment, Zika virus envelope protein E-DIII is selected as the target. In one embodiment, the hepatitis C virus envelope glycoprotein E1 and/or E2 is selected as a target.

In some embodiments, the virus is HCV. The HCV RNA genome encodes a single multimeric protein that cleaves into three structural proteins (core, glycoproteins E1 and E2) and seven non-structural proteins (p7, NS2, NS3, NS4A, NS4B, NS5A and NS5B) upon translation or post-translation. The envelope proteins, glycoproteins E1 and E2, form heterodimers and constitute the viral envelope proteins, which play an important role in mediating viral entry and morphogenesis when the virus enters the host cell. The hepatitis C virus envelope proteoglycans bind to specific proteins on the surface of the host hepatocyte to initiate the entry process. This process involves a large number of host receptors/co-receptors. Wherein, E2 is the major HCV envelope proteoglycan and interacts directly with the receptor/co-receptor. It has long been thought that E1 does not interact directly with the host receptor during this process, but rather that it elicits membrane fusion in conjunction with E2 by maintaining a functional E2 conformation required for receptor binding. In some embodiments, the second target is E1 or E2.

Non-limiting examples of bacterial antigens which may be used as second targets include, but are not limited to, gonorrhea bacterial antigens; pertussis bacterial antigens such as pertussis toxin, filamentous hemagglutinin, pertactin, FIM2, FIM3, adenylate cyclase and other pertussis bacterial antigen components; diptheria bacterial antigens such as diptheria toxin or toxoid and other diphtheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; hemophilus influenza bacterial antigens such as capsular polysaccharides and other hemophilus influenza bacterial antigen components; anthrax bacterial antigens such as anthrax protective antigen and other anthrax bacterial antigen components; rickettsiae bacterial antigens such as romps and other rickettsiae bacterial antigen component. Also included with the bacterial antigens described herein are any other bacterial, mycobacterial, mycoplasmal, rickettsial, or chlamydial antigens. In other embodiments, the second targets bind to bacteria selected from Escherichia coli, Pseudomonas bacteria, Staphylococcal bacteria, Enterobacteriaceae bacteria, Streptococcus bacteria, Haemophilus influenzae, Leptospira interrogans, Legionella bacteria, Mycobacterium tuberculosis, Candida albicans bacteria, Acinetobacter baumannii bacteria, Stenotrophomonas maltophilia bacteria, Clostridium difficile bacteria, Enterococcus bacteria, Klebsiella pneumoniae bacteria, Necrotizing fascitis bacteria, Corynebacterium bacteria, Helicobacter pylori bacteria, Campylobacter bacteria, Salmonellae bacteria, Neisseria gonorrhoeae bacteria, Haemophilus influenza bacteria, Shigella bacteria, and a combination thereof.

Exemplary fungal antigens that may be used as second targets include, but are not limited to, Candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components. Other fungi targets may be from fungal infection comprising Alternaria alternata, Aspergillus fumigatus, Aspergillus niger, Aspergillus flavus, Aspergillus nidulans, Aspergillus paraciticus, Candida albicans, Candida dubliniensis, Candida famata, Candida glabrata, Candida guilliermondii, Candida haemulonii, Candida kejyr, Candida krusei, Candida lusitaniae, Candida norvegensis, Candida parapsilosis, Candida tropicalis, Candida viswanathii, Epidermophyton floccosum, Fusarium graminearum, Fusarium oxysporum, Fusarium solani, Fusarium monoliforme, Trychophyton rubrum, Trychophyton mentagrophytes, Trychophyton inter digitales, Trychophyton tonsurans, Cryptococcus neoformans, Cryptococcus gattii, Cryptococcus grubii, Colletotrichum graminicola, Microsporum canis, Microsporum gypseum, Penicillium marneffei, Tricosporon beigelii, Trichosporon asahii, Trichosporon inkin, Trichosporon asteroides, Trichosporon cutaneum, Trichosporon domesticum, Trichosporon mucoides, Trichosporon ovoides, Trichosporon pullulans, Trichosporon loubieri, Trichosporon japonicum, Scedosporium apiospermum, Scedosporium prolifwans, Paecilomyces variotii, Paecilomyces lilacinus, Acremonium stricutm, Cladophialophora bantiana, Wangiella dermatitidis, Ramichloridium obovoideum, Chaetomium atrobrunneum, Dactlaria gallopavum, Bipolaris spp, Exserohilum rostratum, Absidia corymbifera, Apophysomyces elegans, Mucor indicus, Rhizomucor pusillus, Rhizopus oryzae, Cunninghamella bertholletiae, Cokeromyces recurvatus, Saksenaea vasiformis, Syncephalastrum racemosum, Basidiobolus ranarum, Conidiobolus coronatus Conidiobolus incongruus, Blastomyces dermatitidis, Coccidioides immitis, Coccidioides posadasii, Histoplasma capsulatum, Paracoccidioides brasiliensis, Pseudallescheria boydii, Sporothrix schenckii, Alternaria brassicicola, Alternaria alternata, Aspergillus nidulans, Botrytis cinerea, Cercospora beticola, Cercospora zeae maydis, Cochliobolus heterostrophus, Exserohilum turcicum, Fusarium culmorum, Fusarium oxysporum, Fusarium oxysporum f. sp. dianthi, Fusarium solani, Fusarium pseudograminearum, Fusarium verticilloides, Gaeumannomyces graminis var. tritici, Plasmodiophora brassicae, Sclerotinia sclerotiorum, Stenocarpella (Diplodia) maydis, Thielaviopsis basicola, Verticillium dahliae, Ustilago zeae, Puccinia sorghi, Macrophomina phaseolina, Phialophora gregata, Diaporthe phaseolorum, Cercospora sojina, Phytophthora sojae, Rhizoctonia solani, Phakopsora pachyrhizi, Alternaria macrospora, Cercospora gossypina, Phoma exigua, Puccinia schedonnardii, Puccinia cacabata, Phymatotrichopsis omnivora, Fusarium avenaceum, Alternaria brassicae, Alternaria raphani, Erysiphe graminis (Blumeria graminis), Septoria tritici, Septoria nodorum, Mycosphaerella zeae, Rhizoctonia cerealis, Ustilago tritici, Puccinia graminis, Puccinia triticina, Tilletia indica, Tilletia caries, Tilletia controversa, Alternaria solani, Alternaria brassicae, Alternaria brassicola, Monolinia fructicola, Venturia inaequalis, Cladosporum carpophilum, Botryosphaeria obtuse, Monilinia vaccinia-corymbosi, Sclerotinia homoeocarpa, Podosphaera xanthii, Podosphaera fuliginea, Erysiphe cichoracearum, Blumeria graminis f. sp. Tritici, blumeria graminis f. sp. Hordei, Microsphaera diffusa, Erysiphe necator, Leveillula Taurica, Podosphaera leucotricha, Podosphaera aphanis, Sawadaea tulasnei, Erysiphe berberidis, Golovinomyces orontii, Peronospora belbahrii, Pseudoperonospora cubensis, Plasmopara viticola, Pseudoperonospora humuli, Peronospora manshurica, Plasmopara halstedii, Phytopthora capcisi, Phytopthora infestans, Phytopthora cinnamomic, Phytopthora sojae, Phytopthora agathidicida, Phytopthora cactorum, Phytopthora citricola, Phytopthora fragariae, Phytopthora kernoviae, Phytopthora lateralis, Phytopthora megakarya, Phytopthora multivora, Phytopthora nicotianae, Phytopthora palmivora, Phytopthora ramorum, Phytopthora quercina, and a combination thereof.

Exemplary parasite antigens that may be used as second targets include, but are not limited to, Plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasma antigen components; schistosomae antigens such as glutathione-S-transferase, paramyosin, and other schistosomal antigen components; Leishmania major and other leishmaniae antigens such as gp63, lipophosphoglycan and its associated protein and other leishmanial antigen components; and Trypanosoma cruzi antigens such as the 75-77 kDa antigen, the 56 kDa antigen, and other trypanosomal antigen components.

Exemplary tick antigens that may be used as second targets include, but are not limited to those from the following ticks: haematophagous arthropod such as a tick selected from the group Ixodes, Bothriocrotoninae, Amblyomminae, Haemaphysalinae, Rhipicephalinae (including Hyalomminae), Nuttalliellidae, Argasinae, Otobinae, Antricolinae, Nothhoaspinae and Ornithodorinae; a black-legged tick from the species Ixodes, American Dog Tick (Dermacentor variabillis), Brown Dog Tick (Rhipicephalus sanguineus), or Lone Star Tick (Amblyomma americanum); black-legged tick selected from the group consisting of Ixodes scapulari, Ixodes pacificus, Ixodes ricinus and Ixodes persulcatus.

In some embodiments, the second target is a tumor cell antigen, which may be any molecule whose expression is limited to, or overexpressed in, cancer cells. Numerous tumor associated antigens have been identified and described in the literature and thus they are not all listed herein. In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

In some embodiments, the second target is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvlll), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFI)-I, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin and the A1 domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), as well as any derivate or variant of these surface antigens; differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, pl5; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7; TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, TPS; GPC2, CD276, Delta-like protein ligand 3 (DLL3), NY-ESO-1, melanoma associated antigen 4; survivin protein, synovial sarcoma X breakpoint protein 2, CD3, epidermal growth factor receptor (EGFR), erbb2 tyrosine kinase receptor, HER2, CEA, CD66, CD66e, ROR1, ntrkr1 tyrosine kinase receptor, GPC3, mesothelin, glutamate carboxypeptidase II, PMSA, PD-L1, folate receptor alpha, PSCA, Mucin 1, HLA antigen (such as HLA class I antigen A-2 alpha, HLA class I antigen A-11 alpha, and HLA class II antigen), c-Met, hepatocyte growth factor receptor, K-Ras GTPase (KRAS), IL-15 receptor, Kit tyrosine kinase, PDGF receptor beta, RET tyrosine kinase receptor; Raf 1 protein kinase, Raf B protein kinase, thymidylate synthase, topoisomerase II, Brachyury protein, Flt3 tyrosine kinase, VEGF, VEGF receptor (VEGF-1 receptor, VEGF-2 receptor, and VEGF-3 receptor), estrogen receptor, neoantigen, human papillomavirus E6, and heat shock protein.

### Antibody-Dependent Cellular Phagocytosis in Immune Responses Triggered by Repurposed Antibodies

In some embodiments, passively infused repurposed antibodies may be used in anti-toxin, anti-viral, anti-cancer, and anti-inflammatory treatments. Without wanting to be bound by theory, repurposed antibodies may exert their functions via a multitude of mechanisms. In some embodiments, the repurposed antibodies work by target neutralization, which mainly depends on interaction of the second-target-binding molecule (e.g., Technein) with the second target and are therefore predominantly Fc domain independent. Other embodiments, including antibody-dependent cell-mediated cytotoxicity (ADCC) and antibody-dependent cellular phagocytosis (ADCP), require interactions between the repurposed antibody Fc domain with other proteins or immune effector cells via recognition by Fc receptors. These Fc receptor-dependent antibody functions provide a direct link between the innate and adaptive immune systems, harnessing the potent anti-pathogen functions of the innate immune system, and overcoming its inherent limited pattern recognition capacity by utilizing the diversity and specificity of the adaptive immune response. Fc receptor-dependent antibody functions are important components of the immune response that provide mechanisms for clearance of infected host cells, immune complexes, or opsonized pathogens. Fc receptor-dependent antibody functions are also involved in activation of downstream adaptive immune responses by facilitating antigen presentation or by stimulating the secretion of inflammatory mediators.

In one embodiment, the Fc receptor-dependent function of antibody-dependent cellular phagocytosis (ADCP) provides mechanisms for using the repurposed antibodies in the clearance of pathogens (e.g., virus) and pathogen-infected cells, as well as for stimulation of downstream adaptive immune responses by facilitating antigen presentation, or by stimulating the secretion of inflammatory mediators. In some embodiments, repurposed-antibody-mediated ADCP may be a mechanism of both inhibiting pathogen infection, such as viral infection.

In some embodiments, the antibody is of IgG1 isotype. In some embodiments, the antibody is of IgG2 isotype. In some embodiments, the antibody is of IgG3 isotype. In some embodiments, the antibody is of IgG4 isotype. In some embodiments, the antibody is of IgA1 isotype. In some embodiments, the antibody is of IgA2 isotype. For the human IgG isotype, IgG3 has the highest affinity for most of the type I FcγRs, followed by IgG1, then IgG4, then IgG2. In contrast, subclass is not a predominant source of regulation for ADCP by IgA, as human FcαR has been demonstrated to have similar affinity for IgA1, and IgA2. In some embodiments, the antibody is of the IgA or IgM isotype.

In some embodiments, the antibody is glycosylated. For IgG antibodies, there are 36 possible glycoforms and 4 different subclasses, yielding a total of 144 possible unique Fc regions. Any of these Fc regions may be used in the antibodies of the disclosure.

In some embodiments, the Technein is particularly selected for salient binding to epitopes that engage a repurposed antibody particularly effectively at inducing ADCP. In one embodiment, for ADCP of virions the epitope may be present on the surface of the virions, while for ADCP of infected cells any epitope expressed on the surface of a virus-infected cell may be a potential target. In one embodiment, the Technein is selected for its activity in supporting ADCP of pathogen (e.g., viral) particles. In one embodiment, the Technein is selected for its activity in supporting ADCP of pathogen-infected cells.

In some embodiments, the original antibody is known to mediate ADCP. In some embodiments, the antibody is trastuzumab, rituximab, cetuximab, or ipilimumab.

### Methods of Assessing ADCP

In some embodiments, the ability of the repurposed antibody to mediate ADCP is assayed by the method described in the Examples. In other embodiments, the method is as described in Guillaume Beaudoin-Bussières, Jonathan Richard, Jérémie Prévost, Guillaume Goyette, Andres Finzi, A new flow cytometry assay to measure antibody-dependent cellular cytotoxicity against SARS-COV-2 Spike-expressing cells, STAR Protocols, Volume 2, Issue 4, 2021, 100851. In other embodiment, the method is as described in Duchemin M, Tudor D, Cottignies-Calamarte A, Bomsel M. Antibody-Dependent Cellular Phagocytosis of HIV-1-Infected Cells Is Efficiently Triggered by IgA Targeting HIV-1 Envelope Subunit gp41. Front Immunol. 2020 Jun. 9; 11:1141. doi: 10.3389/fimmu.2020.01141. PMID: 32582208; PMCID: PMC7296124. These references are incorporated herein in their entirety by reference.

### Antibody-Dependent Cellular Cytotoxicity (ADCC)

Antibody-dependent cellular cytotoxicity (ADCC), also called antibody-dependent cell-mediated cytotoxicity, is an immune mechanism through which Fc receptor-bearing effector cells may recognize and kill antibody-coated target cells expressing tumor- or pathogen-derived antigens on their surface. In one embodiment, the antibody is selected for repurposing for its ability to trigger ADCC against tumor cells. In other embodiments, the antibody is selected for repurposing for its ability to trigger ADCC against pathogen-derived antigens on the surface of infected cells. In one embodiment, the original antibody is not capable of ADCC but the repurposed antibody is capable of mediating ADCC. In some embodiments, both antibodies are capable of mediating ADCC.

In some embodiments, the original antibody has been shown to enhance ADCC. Examples of FDA-approved antibodies known to mediate ADCC include trastuzumab (anti-HER2), rituximab (anti-CD20), cetuximab (anti-EGFR), avelumab (anti-PD-L1), ocrelizumab (anti-CD20), Obinutuzumab (anti-CD20), mogamulizumab (anti-CCR4), margetuximab (anti-HER2), ublituximab (anti-CD20).

### Measuring ADCC

In some embodiments, the ability of the repurposed antibody to mediate ADCC is measured by the method described in the Examples. In some other embodiments, ADCC is measured by other methods, selected from labeling of target cells with fluorescent dyes, which allows for sensitive determination of cytotoxicity via flow cytometry (Radošević, K, Garritsen, H, Van Graft, M et al. A simple and sensitive flow cytometric assay for the determination of the cytotoxic activity of human natural killer cells. J Immunol Methods 1968; 135:81-9.); the VITAL assay, which was developed to measure cytotoxicity of multiple target populations simultaneously both in vitro and in vivo (Hermans, I, Silk, J, Yang, J et al. The VITAL assay: a versatile fluorometric technique for assessing CTL- and NKT-mediated cytotoxicity against multiple targets in vitro and in vivo. J Immunol Methods 2004; 285:25-40); the 51Cr release assay but replace radioisotopes with natural cell products; combining flow cytometric and labeling-based techniques for high-throughput single-cell computer image analysis (Welter, A, Sundararaman, S, Li, R et al. High-throughput GLP-capable target cell visualization assay for measuring cell-mediated cytotoxicity. Cells 2018; 7:35); genetically modified target cells to express a reporter protein for a standardized assay (Rossignol, A, Bonnaudet, V, Clémenceau, B et al. A high-performance, non-radioactive potency assay for measuring cytotoxicity: a full substitute of the chromium-release assay targeting the regulatory-compliance objective. MAbs 2017; 9:521-35). Alternatively, as cells are killed they release lactate dehydrogenase and other proteases that may be quantified by supplying fluorogenic substrates in order to more accurately assess cytotoxicity without the need to perform any labeling or manipulation of target cells (Hassenrück, F, Knödgen, E, Göckeritz, E et al. Sensitive detection of the natural killer cell-mediated cytotoxicity of anti-CD20 antibodies and its impairment by B-cell receptor pathway inhibitors. BioMed Res Int 2018; 2018:1-9; Niles, A, Moravec, R, Eric Hesselberth, P et al. A homogeneous assay to measure live and dead cells in the same sample by detecting different protease markers. Anal Biochem 2007; 366:197-206).

Target Blocking and/or Neutralization

In some embodiments, the repurposed antibody is a blocking antibody. In some embodiments, the repurposed antibody is a neutralizing antibody. In some embodiments, a blocking antibody does not trigger a reaction when it binds its target but prevents other molecules (e.g., another antibody) from interfering with the target. In some embodiments, a blocking antibody binds its target and directly interferes with its function, such as blocking cell adhesion or receptor-ligand binding. In some embodiments, a neutralizing antibody binds its target and negates its downstream cellular effects, such as cell proliferation or chemotaxis. In some embodiments, the neutralizing antibody is an endogenous antibody that is produced naturally by the body as part of its immune response to a drug or pathogen. In some embodiments, the antibody (original and/or repurposed antibody) is a neutralizing and/or blocking antibody.

In some embodiments, the original and/or repurposed antibody is a blocking antibody. Non-limiting examples of blocking antibodies include anti-PD-L, anti-PD-L1, and anti-CTLA4 antibodies, including nivolumab, pembrolizumab, ipilimumab, tremelimumab, In some embodiments, the blocking antibody is a complement-blocking antibody. In some embodiments, the antibody binds to and sequesters a plasma protein or a plasma drug. In one embodiment, the antibody is selected from adalimumab, certolizumab pegol, golimumab, infliximab, which bind Tumor Necrosis Factor or bevacizumab, which binds VEGF.

In one embodiment, the disclosure provides neutralizing antibodies, which may be used for virus clearance and to achieve protection against multiple virus They may achieve this in several ways, including interfering with virion binding to receptors, blocking virus uptake into host cells, and preventing uncoating of viral genomes in endosome or causing aggregation of virus particles. In one embodiment, the neutralizing antibody blocks the interaction of the target with its natural receptor. In some embodiments, the neutralizing antibody binds to a virus in a manner that blocks viral infection. In some embodiments, the neutralizing antibody binds to a viral capsid in a manner that inhibits uncoating of the virus genome. In some embodiments, the neutralizing antibody binds to the virus in a manner that it creates viral complexes that may be destroyed by phagocytes.

Accordingly, in some embodiments, an immunotherapeutic agent/compound or composition of the disclosure (i.e., synthetic compound, repurposed antibody, and their compositions) may work in cancer by invoking an immune response to destroy or reduce the incidence of cancer cell growth. In some embodiments, the immunotherapeutic agents of the disclosure may be used in any of the following applications:

Methods to treat or reduce the incidence of a cancer using an immunotherapeutic compound or composition of the disclosure.

In some embodiments, these claims encompass a method of ameliorating, treating, or reducing the incidence of a malignancy in a human subject wherein the steps of the method assist or boost the immune system in eradicating cancerous cells. Examples include:
(a) A method of ameliorating, treating, or reducing the incidence of a cancer/tumor/malignancy in a human subject wherein the steps of the method assist or boost the immune system in eradicating cancerous cells, wherein the method comprises administering a therapeutic effect amount of the antibodies, synthetic compounds, cells, and/or nucleic acids of the disclosure, which that invoke an active (or achieve a passive) immune response to destroy cancerous cells;
b) Methods drawn to the co-administration of biological adjuvants (for example, interleukins, cytokines, Bacillus Calmette-Guerin, monophosphoryl lipid A, etc.) in combination with conventional therapies for treating cancer such as chemotherapy, radiation, or surgery;
c) Methods drawn to the administration of any vaccine, wherein the vaccine works by activating the immune system to destroy or reduce the incidence of cancer cell growth, wherein the vaccine comprises a therapeutic effect amount of the antibodies, synthetic compounds, cells, and/or nucleic acids of the disclosure; and
(d) A method of using a therapeutic effect amount of the antibodies, synthetic compounds, cells, and/or nucleic acids of the disclosure in vivo, ex vivo, as an adoptive immunotherapies for treating a cancer, including using autologous and/or heterologous cells or immortalized cell lines in a subject in need thereof, comprising using or administering the antibodies, synthetic compounds, cells, and/or nucleic acids of the disclosure to a subject and/or contacting the immunotherapeutic cells with a synthetic compound of the disclosure.

(ii) An immunotherapeutic compound or composition of the disclosure, used in a cancer immunotherapy, wherein the compound or composition comprises a repurposed antibody, a fragment thereof, and/or synthetic compound of the disclosure, or a CAR-T, T-cell, or TIL contacted with or conjugated with a compound of the disclosure.

### Indications

As already discussed above, the repurposed antibodies may be used for a variety of indications. In some embodiments, the indications are selected from infectious diseases, cancer, hematologic malignancies, autoimmune disorders, hypercholesterolemia, asthma, osteoporosis, inflammatory bowel disease, allograft rejection, drug reversal. In some embodiments, the repurposed antibody and the original antibody may be used for the same indication, despite having a different target. In some embodiments, the repurposed antibody has gained the ability to bind to, and have the same function as, an antibody selected from those of Tables 1 to 3, because it has been repurposed to bind the same target, even if its original indication was completely different.

In some embodiments, the repurposed antibody is prepared in vitro by combining the original antibody and the synthetic compound(s) of the disclosure in vitro. In some embodiments, the repurposed antibody is prepared in vivo, by administration of the original antibody before, after, and/or concurrently with the synthetic compound(s) of the disclosure.

The repurposed antibodies and/or their components (e.g., antibody, first molecule, second molecule, and synthetic compound) may be administered by any route. In some embodiments, the repurposed antibodies and/or their components are administered via the same route. In some embodiments, the repurposed antibodies and/or their components are administered orally, intravenously, subcutaneously, and/or intramuscularly.

In one embodiment, the repurposed antibodies of the disclosure may be used in treating infections (e.g., viral, bacterial, fungal, etc). In other embodiments, the repurposed antibodies of the disclosure may be used to treat one of more of the following diseases or disorders: Amyotrophic Lateral Sclerosis; endotoxemia; atherosclerotic vascular disease or coronary, cancer (listed elsewhere); rheumatoid arthritis; artery disease; stent restenosis; carotid metabolic disease; stroke; acute myocardial infarction; heart failure; peripheral arterial disease; limb ischemia; vein graft failure; AV fistula failure; Crohn's disease; ulcerative colitis; ileitis and enteritis; vaginitis; psoriasis and inflammatory dermatoses such as dermatitis; eczema; atopic dermatitis; allergic contact dermatitis; urticaria; vasculitis; spondyloarthropathies; scleroderma; respiratory allergic diseases such as asthma; allergic rhinitis; hypersensitivity lung diseases; arthritis (e.g. rheumatoid and psoriatic); eczema; psoriasis; osteoarthritis; multiple sclerosis; systemic lupus erythematosus; diabetes mellitus; glomerulonephritis; graft rejection (including allograft rejection and graft-v-host disease) or rejection of an engineered tissue; infectious diseases; myositis; inflammatory CNS disorders; stroke; closed-head injuries; neurodegenerative diseases; Alzheimer's disease; encephalitis; meningitis; osteoporosis; gout; hepatitis; hepatic veno-occlusive disease (VOD); hemorrhagic cystitis; nephritis; sepsis; sarcoidosis; conjunctivitis; otitis; chronic obstructive pulmonary disease; sinusitis; Bechet's syndrome; graft-versus-tumor effect; mucositis; appendicitis; ruptured appendix; peritonitis; aortic valve disease; mitral valve disease; Rett's syndrome; tuberous sclerosis; phenylketonuria; Smith-Lemli-Opitz syndrome and fragile X syndrome; Parkinson's disease; Aicardi-Goutières Syndrome; Alexander Disease; Allan-Hemdon-Dudley Syndrome; POLG-Related Disorders; Alpha-Mannosidosis (Type II and III); Alström Syndrome; Angelman Syndrome; Ataxia-Telangiectasia; Neuronal Ceroid-Lipofuscinoses; Beta-Thalassemia; Bilateral Optic Atrophy and (Infantile) Optic Atrophy Type 1; Retinoblastoma (bilateral); Canavan Disease; Cerebrooculofacioskeletal Syndrome 1 [COFS1]; Cerebrotendinous Xanthomatosis; Cornelia de Lange Syndrome; MAPT-Related Disorders; Genetic Prion Diseases; Dravet Syndrome; Early-Onset Familial Alzheimer Disease; Friedreich Ataxia [FRDA]; Fryns Syndrome; Fucosidosis; Fukuyama Congenital Muscular Dystrophy; Galactosialidosis; Gaucher Disease; Organic Acidemias; Hemophagocytic Lymphohistiocytosis; Hutchinson-Gilford Progeria Syndrome; Mucolipidosis II; Infantile Free Sialic Acid Storage Disease; PLA2G6-Associated Neurodegeneration; Jervell and Lange-Nielsen Syndrome; Junctional Epidermolysis Bullosa; Huntington Disease; Krabbe Disease (Infantile); Mitochondrial DNA-Associated Leigh Syndrome and NARP; Lesch-Nyhan Syndrome; LIS1-Associated Lissencephaly; Lowe Syndrome; Maple Syrup Urine Disease; MECP2 Duplication Syndrome; ATP7A-Related Copper Transport Disorders; LAMA2-Related Muscular Dystrophy; Arylsulfatase A Deficiency; Mucopolysaccharidosis Types I; II or III; Peroxisome Biogenesis Disorders; Zellweger Syndrome Spectrum; Neurodegeneration with Brain Iron Accumulation Disorders; Acid Sphingomyelinase Deficiency; Niemann-Pick Disease Type C; Glycine Encephalopathy; ARX-Related Disorders; Urea Cycle Disorders; COL1A½-Related Osteogenesis Imperfecta; Mitochondrial DNA Deletion Syndromes; PLP1-Related Disorders; Perry Syndrome; Phelan-McDermid Syndrome; Glycogen Storage Disease Type II (Pompe Disease) (Infantile); MAPT-Related Disorders; MECP2-Related Disorders; Rhizomelic Chondrodysplasia Punctata Type 1; Roberts Syndrome; Sandhoff Disease; Schindler Disease-Type 1; Adenosine Deaminase Deficiency; Smith-Lemli-Opitz Syndrome; Spinal Muscular Atrophy; Infantile-Onset Spinocerebellar Ataxia; Hexosaminidase A Deficiency; Thanatophoric Dysplasia Type 1; Collagen Type VI-Related Disorders; Usher Syndrome Type I; Congenital Muscular Dystrophy; Wolf-Hirschhom Syndrome; Lysosomal Acid Lipase Deficiency; and Xeroderma Pigmentosum.

### Pharmaceutical Compositions and Formulations

Provided herein are compositions comprising an original antibody and/or a synthetic molecule of the disclosure (or a part thereof), a repurposed antibody or a target-binding fragment thereof, as described herein, and another component, such as a carrier. Also provided are pharmaceutical compositions/therapeutic formulations comprising an original antibody and/or a synthetic molecule of the disclosure (or a part thereof), a repurposed antibody or a target-binding fragment thereof, as described herein, and an excipient and/or diluent. In one embodiment, the carrier is not a naturally existing compound. In one embodiment, the excipient is not a naturally existing compound. In another embodiment, the diluent is not a naturally existing compound. In another embodiment, the formulation comprising the repurposed antibody or a target-binding fragment thereof, as described herein, does not contain a naturally existing compound, except, optionally, water. It will be apparent to those persons skilled in the art that certain carriers, excipients, or diluents may be more preferable depending upon, for instance, the route of administration and concentration of antibody being administered.

In one embodiment, therapeutic formulations of the original antibody and/or a synthetic molecule of the disclosure (or a part thereof), a repurposed antibody or a target-binding fragment thereof used in accordance with the present disclosure are prepared for storage and/or administration by mixing an original antibody and/or a synthetic molecule of the disclosure (or a part thereof), a repurposed antibody or a target-binding fragment thereof having the desired degree of purity with optional pharmaceutically acceptable carriers, diluents, excipients or stabilizers (Remington's Pharmaceutical Sciences 16th edition, Osol, A. Ed. (1980)), in the form of lyophilized formulations or aqueous solutions. In one embodiment, acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN, PLURONICS or polyethylene glycol (PEG). Examples of lyophilized antibody formulations are described in WO 97/04801, expressly incorporated herein by reference.

In a further embodiment, the formulation further comprises a surfactant. The surfactant may, for example, be selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof. C6-C12 (eg. oleic acid and caprylic acid), acylcarnitines and derivatives, N alpha.-acylated derivatives of lysine, arginine or histidine, or side-chain acylated derivatives of lysine or arginine, Nalpha.-acylated derivatives of dipeptides comprising any combination of lysine, arginine or histidine and a neutral or acidic amino acid, Nalpha-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N, N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl .beta.-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. In one embodiment, the surfactant is not a naturally existing compound. Each one of these specific surfactants constitutes an alternative embodiment of the disclosure.

One embodiment provides for stable formulations of an original antibody and/or a synthetic molecule of the disclosure (or a part thereof), a repurposed antibody or a target-binding fragment thereof, which comprise preferably a phosphate buffer with saline or a chosen salt, as well as preserved solutions and formulations containing a preservative, as well as multi-use preserved formulations suitable for pharmaceutical or veterinary use, comprising at least one the antibodies and/or target-binding fragments thereof in a pharmaceutically acceptable formulation. In one embodiment, preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture may be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3, 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like. In one embodiment, the preservative or preservatives are not naturally existing compounds.

In one embodiment, an original antibody and/or a synthetic molecule of the disclosure (or a part thereof), a repurposed antibody or a target-binding fragment thereof of the disclosure may be incorporated into pharmaceutical compositions suitable for administration to a subject. In one common embodiment, the pharmaceutical composition comprises an original antibody and/or a synthetic molecule of the disclosure (or a part thereof), a repurposed antibody or a target-binding fragment thereof of the disclosure and a pharmaceutically acceptable carrier. In one embodiment, “pharmaceutically acceptable carrier” includes any and all solvents, dispersion media, coatings, antibacterial and antifungal agents, isotonic and absorption delaying agents, and the like that are physiologically compatible. Additional examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or target-binding fragment thereof.

In one embodiment, an appropriate amount of a pharmaceutically-acceptable salt is used in the formulation to render the formulation isotonic. Examples of the carrier include saline, Ringer's solution and dextrose solution. In one embodiment, the pH of the solution is from about 5 to about 8. In another embodiment, the pH is from about 7 to about 7.5. Further carriers include sustained release preparations such as semipermeable matrices of solid hydrophobic polymers containing the antibody or target-binding fragment thereof, which matrices are in the form of shaped articles, e.g., films, liposomes or microparticles. A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials such as liposomes, polylactides (polylactide acid), polyglycolide (polymer of glycolic acid), polylactide co-glycolide (copolymers of lactic acid and glycolic acid), polyanhydrides, poly (ortho) esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The compositions of this disclosure may be in a variety of forms. These include, for example, liquid, semi-solid and solid dosage forms, such as liquid solutions (e.g., injectable and infusible solutions), dispersions or suspensions, tablets, pills, powders, liposomes and suppositories. In some embodiments, such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present disclosure may be formulated as a lyophilizate. An original antibody and/or a synthetic molecule of the disclosure (or a part thereof), a repurposed antibody or a target-binding fragment thereof may also be encapsulated within liposomes using well-known technologies.

Dosage forms suitable for internal administration generally contain from about 0.1 milligram to about 500 milligrams of antibody or target-binding fragment thereof (the active ingredient) per unit or container. In these pharmaceutical compositions, the active ingredient will ordinarily be present in an amount of about 0.5-99.999% by weight based on the total weight of the composition.

Therapeutic compositions/formulations typically must be sterile and stable under the conditions of manufacture and storage. The composition may be formulated as a solution, microemulsion, dispersion, liposome, or other ordered structure suitable to high drug concentration. Sterile injectable solutions may be prepared by incorporating the active compound (i.e., antibody or target-binding fragment thereof) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution may be maintained, for example, by the use of a coating such as lecithin, by the maintenance of the required particle size in the case of dispersion and by the use of surfactants. Prolonged absorption of injectable compositions may be brought about by including in the composition an agent that delays absorption, for example, monostearate salts and gelatin.

The preferred dosage form depends on the intended mode of administration and therapeutic application. One of ordinary skill in the art is familiar with the procedures for determining such dosages. Typical compositions are in the form of injectable or infusible solutions, such as compositions similar to those used for passive immunization of humans with other antibodies. The most typical mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

The original antibody and/or a synthetic molecule of the disclosure (or a part thereof), a repurposed antibody or a target-binding fragment thereof of the present disclosure may be administered by a variety of methods known in the art, although for many therapeutic applications, the preferred route/mode of administration is intravenous injection or infusion. As will be appreciated by the skilled artisan, the route and/or mode of administration will vary depending upon the desired results. In certain embodiments, an original antibody and/or a synthetic molecule of the disclosure (or a part thereof), a repurposed antibody or a target-binding fragment thereof may be prepared with a carrier that will protect the compound against rapid release, such as a controlled release formulation, including implants, transdermal patches, and microencapsulated delivery systems. Biodegradable, biocompatible polymers may be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an original antibody and/or a synthetic molecule of the disclosure (or a part thereof), a repurposed antibody or a target-binding fragment thereof of the disclosure may be orally administered, for example, with an inert diluent or an assimilable edible carrier. The original antibody and/or a synthetic molecule of the disclosure (or a part thereof), a repurposed antibody or a target-binding fragment thereof (and other ingredients, if desired) may also be enclosed in a hard or soft shell gelatin capsule, compressed into tablets, or incorporated directly into the subject's diet. For oral therapeutic administration, an original antibody and/or a synthetic molecule of the disclosure (or a part thereof), a repurposed antibody or a target-binding fragment thereof may be incorporated with excipients and used in the form of ingestible tablets, buccal tablets, troches, capsules, elixirs, suspensions, syrups, wafers, and the like. To administer an original antibody and/or a synthetic molecule of the disclosure (or a part thereof), a repurposed antibody or a target-binding fragment thereof of the disclosure by other than parenteral administration, it may be necessary to coat the antibody or target-binding fragment thereof with, or co-administer the antibody or target-binding fragment thereof with, a material to prevent its inactivation.

In certain embodiments, the therapeutically effective amount of a repurposed antibody is determined as an amount provided in a package insert provided with the original antibody. The term package insert refers to instructions customarily included in commercial packages of medicaments approved by the FDA or a similar regulatory agency of a country other than the USA, which contains information about, for example, the usage, dosage, administration, contraindications, and/or warnings concerning the use of such medicaments.

The repurposed antibody or a target-binding fragment thereof may be administered in an amount as a measure with regards to the weight of the patient in need thereof. In one embodiment, the repurposed antibody or a target-binding fragment thereof may be administered in an amount of about: 0.1 mg/kg to about 50 mg/kg, 0.1 mg/kg to about 40 mg/kg, 0.1 mg/kg to about 30 mg/kg, 0.1 mg/kg to about 25 mg/kg, 0.1 mg/kg to about 20 mg/kg, 0.1 mg/kg to about 15 mg/kg, 0.1 mg/kg to about 10 mg/kg, 0.1 mg/kg to about 7.5 mg/kg, 0.1 mg/kg to about 5 mg/kg, 0.1 mg/kg to about 2.5 mg/kg, or about 0.1 mg/kg to about 1 mg/kg. The repurposed antibody or a target-binding fragment thereof may be administered in an amount of about: 0.5 mg/kg to about 50 mg/kg, 0.5 mg/kg to about 40 mg/kg, 0.5 mg/kg to about 30 mg/kg, 0.5 mg/kg to about 25 mg/kg, 0.5 mg/kg to about 20 mg/kg, 0.5 mg/kg to about 15 mg/kg, 0.5 mg/kg to about 10 mg/kg, 0.5 mg/kg to about 7.5 mg/kg, 0.5 mg/kg to about 5 mg/kg, 0.5 mg/kg to about 2.5 mg/kg, or about 0.5 mg/kg to about 1 mg/kg. The repurposed antibody or a target-binding fragment thereof may be administered in an amount of about 0.5 mg/kg to about 5 mg/kg or about 0.1 mg/kg to about 10 mg/kg. The repurposed antibody or a target-binding fragment thereof may be administered in an amount of about 0.1 mg/kg to about 20 mg/kg or about 0.1 mg/kg to about 30 mg/kg.

The repurposed antibody or a target-binding fragment thereof may be administered at an amount of about: 1 mg, 5 mg, 10 mg, 15 mg, 20 mg, 25 mg, 30 mg, 40 mg, 50 mg, 60 mg, 70 mg, 75 mg, 80 mg, 90 mg, 100 mg, 150 mg, or 200 mg. The repurposed antibody or a target-binding fragment thereof may be administered at an amount of about: 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg. The repurposed antibody or a target-binding fragment thereof may be administered at an amount of about 1000 mg to about 2000 mg. The repurposed antibody or a target-binding fragment thereof may be administered at an amount of about: 1 mg to about 10 mg, 10 mg to about 20 mg, 25 mg to about 50 mg, 30 mg to about 60 mg, 40 mg to about 50 mg, 50 mg to about 100 mg, 75 mg to about 150 mg, 100 mg to about 200 mg, 200 mg to about 500 mg, 500 mg to about 1000 mg, 1000 mg to about 1200 mg, 1000 mg to about 1500 mg, 1200 mg to about 1500 mg, or 1500 to about 2000 mg.

The repurposed antibody or a target-binding fragment thereof may be administered in an amount of about 0.1 mg/ml, 0.5 mg/ml, 1 mg/mL, 2 mg/ml, 3 mg/mL, 4 mg/mL, 5 mg/mL, 6 mg/ml, 7 mg/mL, 8 mg/mL, 9 mg/mL, 10 mg/mL, 15 mg/mL, 20 mg/mL, 25 mg/ml, 30 mg/mL, 40 mg/mL, 50 mg/mL, 60 mg/mL, 70 mg/mL, 80 mg/mL, 90 mg/mL, 100 mg/mL, 150 mg/mL, 200 mg/ml, 250 mg/mL, 300 mg/mL, 400 mg/mL, or 500 mg/mL. In one embodiment, the repurposed antibody or a target-binding fragment thereof is present in the combination in an amount of about: 1 mg/ml to about 10 mg/mL, 5 mg/mL to about 10 mg/mL, 5 mg/ml to about 15 mg/ml, 10 mg/ml to about 25 mg/ml; 20 mg/ml to about 30 mg/ml; 25 mg/ml to about 50 mg/ml, or 50 mg/ml to about 100 mg/mL.

The first molecule and/or second molecule may be administered, for example, once a day (QD), twice daily (BID), once a week (QW), twice weekly (BIW), three times a week (TIW), or monthly (QM) regularly on a continuous base or intermittent base such as BIW for 3 months then resume a month later. In one embodiment, the first molecule and/or second molecule may be administered BID. The first molecule and/or second molecule may be administered TIW. In certain instances, the first molecule and/or second molecule is administered 2 to 3 times a week. In another embodiment, the first molecule and/or second molecule is administered QD. The compound may be administered QD for about: 1 day to about 7 days, 1 day to about 14 days, 1 day to about 21 days, 1 day to about 28 days, or daily until disease progression or unacceptable toxicity. The administration of a first molecule and/or second molecule and/or synthetic compound may, in part, depend upon the tolerance of the patient where greater tolerance may allow greater or more frequent administration. Alternatively, where a patient shows poor tolerance to a first molecule and/or second molecule and/or synthetic compound, a less amount of the compound or a less frequent dosing may be performed. Compounds of formula I may be administered in any regimen as described herein.

In one embodiment, a first molecule and/or second molecule and/or synthetic compound may be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QD. In one embodiment, a first molecule and/or second molecule and/or synthetic compound may be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, BIW. In one embodiment, a first molecule and/or second molecule and/or synthetic compound may be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, TIW. In one embodiment, a first molecule and/or second molecule and/or synthetic compound may be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QW. In one embodiment, a first molecule and/or second molecule and/or synthetic compound may be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, Q2W. In one embodiment, a first molecule and/or second molecule and/or synthetic compound may be administered at an amount of about 5 mg or about 10 mg, QD. In one embodiment, a first molecule and/or second molecule and/or synthetic compound may be administered at an amount of about 5 mg or about 10 mg, BIW. In one embodiment, a first molecule and/or second molecule and/or synthetic compound may be administered at an amount of about 5 mg or about 10 mg, TIW. In one embodiment, a first molecule and/or second molecule and/or synthetic compound may be administered at an amount of about 5 mg or about 10 mg, QW. In one embodiment, a first molecule and/or second molecule and/or synthetic compound may be administered at an amount of about 5 mg or about 10 mg, Q2W. Administration of a first molecule and/or second molecule and/or synthetic compound may be continuous. Administration of a first molecule and/or second molecule and/or synthetic compound may be intermittent.

In one embodiment, a first molecule and/or second molecule and/or synthetic compound may be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, QD. In one embodiment, a first molecule and/or second molecule and/or synthetic compound may be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, BIW. In one embodiment, a first molecule and/or second molecule and/or synthetic compound may be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, TIW. In one embodiment, a first molecule and/or second molecule and/or synthetic compound may be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, QW. In one embodiment, a first molecule and/or second molecule and/or synthetic compound may be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, Q2W. Administration of a first molecule and/or second molecule and/or synthetic compound may be continuous. Administration of a first molecule and/or second molecule and/or synthetic compound may be intermittent.

In one embodiment, a first molecule and/or second molecule and/or synthetic compound may be administered at an amount of about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QD. In one embodiment, a first molecule and/or second molecule and/or synthetic compound may be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, BIW. In one embodiment, a first molecule and/or second molecule and/or synthetic compound may be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, TIW. In one embodiment, a first molecule and/or second molecule and/or synthetic compound may be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QW. In one embodiment, a first molecule and/or second molecule and/or synthetic compound may be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, Q2W. In one example, a first molecule and/or second molecule and/or synthetic compound may be administered at an amount of about 15 mg/kg to about 75 mg/kg, QD. In another example, a first molecule and/or second molecule and/or synthetic compound may be administered at an amount of about 20 mg/kg to about 50 mg/kg. In still another example, a first molecule and/or second molecule and/or synthetic compound may be administered at an amount of about 0.001 mg/kg, 0.01 mg/kg, 0.05 mg/kg, 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 40 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. Administration of a first molecule and/or second molecule and/or synthetic compound may be continuous. Administration of a first molecule and/or second molecule and/or synthetic compound may be intermittent.

In one embodiment, a first molecule and/or second molecule and/or synthetic compound may be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, QD. In one embodiment, a first molecule and/or second molecule and/or synthetic compound may be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, BIW. In one embodiment, a first molecule and/or second molecule and/or synthetic compound may be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, TIW. In one embodiment, a first molecule and/or second molecule and/or synthetic compound may be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, QW. In one embodiment, a first molecule and/or second molecule and/or synthetic compound may be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, Q2W. In one example, a first molecule and/or second molecule and/or synthetic compound may be administered at an amount of about 15 mg/kg to about 75 mg/kg, QD. In another example, a first molecule and/or second molecule and/or synthetic compound may be administered at an amount of about 20 mg/kg to about 50 mg/kg. In still another example, a first molecule and/or second molecule and/or synthetic compound may be administered at an amount of about 0.001 mg/kg, 0.01 mg/kg, 0.05 mg/kg, 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 40 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. Administration of a first molecule and/or second molecule and/or synthetic compound may be continuous. Administration of a first molecule and/or second molecule and/or synthetic compound may be intermittent.

The original antibody and/or a synthetic molecule of the disclosure (or a part thereof), a repurposed antibody or a target-binding fragment thereof, or any one of the combinations described herein may be administered in a regimen. The regimen may be structured to provide therapeutically effective amounts of an original antibody and/or a synthetic molecule of the disclosure (or a part thereof), a repurposed antibody or a target-binding fragment thereof, or any one of the combinations described herein, over a predetermined period of time (e.g., an administration time). The regimen may be structured to limit or prevent side-effects or undesired complications of each of the components of an original antibody and/or a synthetic molecule of the disclosure (or a part thereof), a repurposed antibody or a target-binding fragment thereof, or any one of the combinations described herein described herein. The regimen may be structured in a manner that results in increased effect for both therapies of the combination (e.g., synergy). Regimens useful for treating cancer may include any number of days of administration which may be repeated as necessary. Administration periods may be broken by a rest period that includes no administration of at least one therapy. For example, a regimen may include administration periods that include 2, 3, 5, 7, 10, 15, 21, 28, or more days. These periods may be repeated. For example, a regimen may include a set number of days as previously described where the regimen is repeated 1, 2, 3, 4, 5, 6, 7, 8, 9, 10, 11, 12, 13, or more times.

In other embodiments, the regimens may include a rest period of at least 1, 2, 3, 5, 7, 10, or more days, where at least one therapy is no longer administered to a patient. The rest period may be determined by, for example, monitoring the reaction of the patient to an original antibody and/or a synthetic molecule of the disclosure (or a part thereof), a repurposed antibody or a target-binding fragment thereof, or any one of the combinations described herein or by measuring the efficacy of the treatment. A rest period may be applicable to a single therapy, such that only one therapy of a combination described herein is discontinued in the rest period but the other therapy(ies) are still administered. Rest periods may be applied to all of the therapies administered to the subject such that the subject receives no therapy for a set period of time during the rest period.

### Combination Treatments

In some embodiments, the original antibody and/or a synthetic molecule of the disclosure (or a part thereof), a repurposed antibody or a target-binding fragment thereof, may be formulated with another therapeutic agent. This agent may be anything that may also be used in the indication for which the repurposed antibody is designed to work.

In some embodiments, the original antibody and/or a synthetic molecule of the disclosure (or a part thereof), a repurposed antibody or a target-binding fragment thereof, may be co-administered in combination with another therapeutic agent or treatment (e.g., radiation). In some embodiments, the therapeutic agent or treatment are administered before, concurrently, and/or after the administration of the original antibody and/or a synthetic molecule of the disclosure (or a part thereof), a repurposed antibody or a target-binding fragment thereof. “Co-administered,” as used herein, means that two (or more) different treatments (e.g., a repurposed antibody and an antibiotic) are delivered to the subject during the course of the subject's affliction with the disease, e.g., the two or more treatments are delivered after the subject has been diagnosed with a disease and before the disease has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom (e.g., toxicity resulting from by administration of repurposed antibody), or other parameter related to the disorder is greater than what would be observed with one treatment delivered in the absence of the other. The effect of the two treatments may be partially additive, wholly additive, or greater than additive. The delivery may be such that an effect of the first treatment delivered is still detectable when the second is delivered. The molecules of the disclosure and the additional therapeutic agent may be administered simultaneously, in the same or in separate compositions, or sequentially. In one embodiment, for sequential administration, the repurposed antibodies or other molecules described herein may be administered first, and the therapeutic agent may be administered second, or the order of administration may be reversed. The repurposed antibody or any other molecule described herein may be administered during periods of active disorder, or during a period of remission or less active disease. The repurposed antibody or any other molecule described herein may be administered before another treatment, concurrently with the treatment, post-treatment, or during remission of the disorder (e.g., cancer).

In one embodiment, the other therapeutic agent comprises an agent that works as an antibiotic, antiviral, anti-inflammatory agent, a cytokine, a hematopoietic growth factor, anti-cancer agent (including a chemotherapeutic agent), an immunomodulatory agent, an immunosuppressive agent, a steroid (e.g., a corticosteroid) or a pharmacologically active derivative thereof, a therapeutic antibody, a vitamin, calcium or a calcium supplement.

In some embodiments, the therapeutic agent is another antibody. In some embodiments, the other antibody is selected from those listed in Table 1.

In one embodiment, the therapeutic agent an antibiotic selected Amikacin, Amoxicillin, Amoxicillin-clavulanic acid, Amphothericin-B, Ampicillin, Ampicllin-sulbactam, Apramycin, Azithromycin, Aztreonam, Bacitracin, Benzylpenicillin, Caspofungin, Cefaclor, Cefadroxil, Cefalexin, Cefalothin, Cefazolin, Cefdinir, Cefepime, Cefixime, Cefmenoxime, Cefoperazone, Cefoperazone-sulbactam, Cefotaxime, Cefoxitin, Cefbirome, Cefpodoxime, Cefpodoxime-clavulanic acid, Cefpodoxime-sulbactam, Cefbrozil, Cefquinome, Ceftazidime, Ceftibutin, Ceftiofur, Ceftobiprole, Ceftriaxon, Cefuroxime, Chloramphenicole, Florfenicole, Ciprofloxacin, Clarithromycin, Clinafloxacin, Clindamycin, Cloxacillin, Colistin, Cotrimoxazol (Trimthoprim/sulphamethoxazole), Dalbavancin, Dalfopristin/Quinopristin, Daptomycin, Dibekacin, Dicloxacillin, Doripenem, Doxycycline, Enrofloxacin, Ertapenem, Erythromycin, Flucloxacillin, Fluconazol, Flucytosin, Fosfomycin, Fusidic acid, Garenoxacin, Gatifloxacin, Gemifloxacin, Gentamicin, Imipenem, Itraconazole, Kanamycin, Ketoconazole, Levofloxacin, Lincomycin, Linezolid, Loracarbef, Mecillnam (amdinocillin), Meropenem, Metronidazole, Meziocillin, Mezlocillin-sulbactam, Minocycline, Moxifloxacin, Mupirocin, Nalidixic acid, Neomycin, Netilmicin, Nitrofurantoin, Norfloxacin, Ofloxacin, Oxacillin, Pefloxacin, Penicillin V, Piperacillin, Piperacillin-sulbactam, Piperacillin-tazobactam, Rifampicin, Roxythromycin, Sparfloxacin, Spectinomycin, Spiramycin, Streptomycin, Sulbactam, Sulfamethoxazole, Teicoplanin, Telavancin, Telithromycin, Temocillin, Tetracyklin, Ticarcillin, Ticarcillin-clavulanic acid, Tigecycline, Tobramycin, Trimethoprim, Trovafloxacin, Tylosin, Vancomycin, Virginiamycin, Voriconazole, and combinations thereof.

In one embodiment, the antiviral is selected from remdesivir, oseltamivir phosphate, zanamivir, peramivir, baloxavir marboxil, darunavir, atazanavir, ritonavir, acyclovir, valacyclovir, valganciclovir, tenofovir, raltegravir, viral attachment inhibitors, viral entry inhibitors, uncoating inhibitors, protease inhibitors, polymerase inhibitors, nucleoside and nucleotide reverse transcriptase inhibitors, nonnucleoside reverse-transcriptase inhibitors, integrase inhibitors; nucleoside analogs (e.g., zidovudine, acyclovir, gancyclovir, vidarabine, idoxuridine, trifluridine, and ribavirin), foscarnet, amantadine, peramivir, rimantadine, saquinavir, indinavir, ritonavir, alpha-interferons and other interferons, AZT, t-705, zanamivir (Relenza®), and oseltamivir (Tamiflu®). Other anti-viral agents include influenza virus vaccines, e.g., Fluarix® (Glaxo SmithKline), FluMist® (MedImmune Vaccines), Fluvirin® (Chiron Corporation), Flulaval® (GlaxoSmithKline), Afluria® (CSL Biotherapies Inc.), Agriflu® (Novartis), Fluzone® (Aventis Pasteur), Remdesivir, lopinavir, Favipiravir, Darunavir, Oseltamivir, Umifenovir, Novaferon; immune modulating drug selected from immunoglobulins, monoclonal antibodies; glucocorticoid; anti-inflammatory drug selected from leflunomide, colchicine, naproxen, piclidenoson; cardiovascular drugs selected from ACE-2, ACE-inhibitor, ARB, angiotensin; Chloroquine; Hydroxychloroquine; Aviptadil, Azithromycin; Itraconazole; zidovudine, acyclovir, gancyclovir, vidarabine, idoxuridine, trifluridine, ribavirin, foscarnet, amantadine, peramivir, rimantadine, saquinavir, indinavir, ritonavir, alpha-interferons, AZT, t-705, zanamivir, oseltamivir; Vitamin D; zinc; and combinations thereof.

In one embodiment, the therapeutic agent is an anti-SARS-COV-2 vaccine selected from the group consisting of an intranasal SARS-COV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-COV-2 spike protein antigen; Oxford-AstraZeneca), Covishield (ChAdOx1_nCoV19) recombinant ChAdOx1 adenoviral vector encoding SARS-COV-2 spike protein antigen (Serum Institute of India), SARS-COV-2 Vaccine (Vero Cell), Inactivated (InCoV) (Sinopharm/BIBP), SARS-COV-2 Vaccine (Vero Cell), Inactivated (Sinovac), Ad26.COV2.S recombinant, replication-incompetent adenovirus type 26 (Ad26) vectored vaccine encoding SARS-COV-2) Spike(S) protein (Janssen Pharmaceuticals Companies of Johnson & Johnson), Sputnik V Human Adenovirus Vector-based Covid-19 vaccine (The Gamaleya National Center), Ad5-nCOV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-COV-2 Vaccine, Inactivated (Vero Cell) (IMBCAMS, China), Inactivated SARS-COV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-COV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), SARS-Cov-2 coronavirus vaccine NVX-CoV2373 (Novavax), an intramuscular vaccine INO-4700 (GLS-5300) (Inovio Pharma and GeneOne Life Science), and combinations thereof.

In one embodiment, the therapeutic agent is a SARS-COV-2 RNA polymerase inhibitor, a serine protease inhibitor, a cysteine protease inhibitor, galidesivir, remdisivir, hydrochloroquine, chloroquine, irbesartan, toremifene, camphor, equiline, mesalazine, mercaptopurine, nafamostat, paraoxetine, sirolimus, carvedilol, dactinomycin, melatonin, quinacrine, eplerenone, enoclin, oxymethalone, ENU2000, azithromycin, lopinovir/ritonavir, umifenovir, cytovene, ganciclovir, trisodium phosphonoformate, ribavirin, interferon, d4T, ddl, AZT, amantadine, rimantadine, acyclovir, foscamet, laninamivir, oseltamivir, zanamivir, favipiravir, baloxavir marboxil, and peramivir, or a combination thereof.

In one embodiment, the therapeutic agent is a “check point inhibitor.” The term “check-point inhibitor” refers to molecules that totally or partially reduce, inhibit, interfere with or modulate one or more checkpoint proteins. Without being limited by a particular theory, checkpoint proteins regulate T-cell activation or function. Numerous checkpoint proteins are known, such as CTLA-4 and its ligands CD80 and CD86; and PD-1 with its ligands PD-L1 and PD-L2. These proteins appear responsible for co-stimulatory or inhibitory interactions of T-cell responses. Immune check-point proteins appear to regulate and maintain self-tolerance and the duration and amplitude of physiological immune responses.

In one embodiment, the therapeutic agent is one or more immune cells expressing one or more chimeric antigen receptors (CARs) on their surface (e.g., a modified immune cell). Generally, CARs comprise an extracellular domain from a first protein e.g., an antigen-binding protein), a transmembrane domain, and an intracellular signaling domain. In certain embodiments, once the extracellular domain binds to a target protein such as a tumor-associated antigen (TAA) or tumor-specific antigen (TSA), a signal is generated via the intracellular signaling domain that activates the immune cell, e.g., to target and kill a cell expressing the target protein. In some embodiments, the therapeutic agent may be with one or more immune cells expressing one or more chimeric antigen receptors (CARs) on their surface (e.g., a modified immune cell). Generally, CARs comprise an extracellular domain from a first protein e.g., an antigen-binding protein), a transmembrane domain, and an intracellular signaling domain. In certain embodiments, once the extracellular domain binds to a target protein such as a tumor-associated antigen (TAA) or tumor-specific antigen (TSA), a signal is generated via the intracellular signaling domain that activates the immune cell, e.g., to target and kill a cell expressing the target protein. The modified immune cells expressing the CARs may be, e.g., T lymphocytes (T cells, e.g., CD4+ T cells or CD8+ T cells), cytotoxic lymphocytes (CTLs) or natural killer (NK) cells. T lymphocytes used in the compositions and methods provided herein may be naïve T lymphocytes or MHC-restricted T lymphocytes. In certain embodiments, the T lymphocytes are tumor infiltrating lymphocytes (TILs). In certain embodiments, the T lymphocytes have been isolated from a tumor biopsy, or have been expanded from T lymphocytes isolated from a tumor biopsy. In certain other embodiments, the T cells have been isolated from, or are expanded from T lymphocytes isolated from, peripheral blood, cord blood, or lymph. Immune cells to be used to generate modified immune cells expressing a CAR may be isolated using art-accepted, routine methods, e.g., blood collection followed by apheresis and optionally antibody-mediated cell isolation or sorting. The modified immune cells are preferably autologous to an individual to whom the modified immune cells are to be administered. In certain other embodiments, the modified immune cells are allogeneic to an individual to whom the modified immune cells are to be administered. Where allogeneic T lymphocytes or NK cells are used to prepare modified T lymphocytes, it is preferable to select T lymphocytes or NK cells that will reduce the possibility of graft-versus-host disease (GVHD) in the individual. For example, in certain embodiments, virus-specific T lymphocytes are selected for preparation of modified T lymphocytes; such lymphocytes will be expected to have a greatly reduced native capacity to bind to, and thus become activated by, any recipient antigens. In certain embodiments, recipient-mediated rejection of allogeneic T lymphocytes may be reduced by co-administration to the host of one or more immunosuppressive agents, e.g., cyclosporine, tacrolimus, sirolimus, cyclophosphamide, or the like.

In one embodiment, the chemotherapeutic agent is selected from acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; clofarabine; crisnatol mesylate; cyclophosphamide; Ara-C; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; omacetaxine; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sorafenib; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs to be included within the methods herein include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; Ara-C ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec); imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen) (Genasense®; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosane polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

In one embodiment, the additional therapeutic agent is a steroid. One of ordinary skill in the art would understand that steroids have various medical uses, including but not limited to: (1) anti-inflammatory uses, e.g. betamethasone, budesonide, cortisone, dexamethasone, hydrocortisone, methylprednisolone, prednisolone, prednisone, and triamcinolone; (2) antiemetic uses, e.g. dexamethasone, hydrocortisone, and prednisone; (3) diagnostic uses, e.g. dexamethasone, as used to detect Cushing's syndrome; and (4) immunosuppressant uses, e.g. betamethasone, cortisone, dexamethasone, hydrocortisone, methylprednicolone, prednisolone, prednisone, and triamcinolone. Moreover, one of ordinary skill in the art would understand that corticosteroid drugs may be used as ingredients contained in eye products (to treat various eye conditions), inhalers (to treat asthma or bronchial disease), nasal drops and sprays (to treat various nasal conditions), and topical products such as ointments and creams (to treat various skin conditions).

While various specific embodiments/aspects have been illustrated and described, it will be appreciated that various changes can be made without departing from the spirit and scope of the disclosure.

All publications, patents, patent applications and other documents cited in this application are hereby incorporated by reference in their entireties for all purposes to the same extent as if each individual publication, patent, patent application or other document were individually indicated to be incorporated by reference for all purposes.

### EXAMPLES

### Antibody Repurposing

Antibody repurposing may be done for multiple applications. In one example, FDA-approved antibodies may be repurposed to safely clear biothreat pathogens using the body's immune system. There are many advantages of this approach: Utilize highly-optimized, safe, and low-cost antibodies to accelerate drug development; Ability to rapidly generate Technein repurposing agents would enable response time to new infectious agents: <6 months instead of years; Platform technology with multiple product outputs. Repurposing health regulatory agency- (e.g., FDA) approved drugs may enable shorter approval.

### Example 1

### Identification of New Techneins Against Sars-Cov-2 Spike Protein

A DHQ10 library was screened for Techneins that bind to SARS-COV-2 Spike protein (i.e., a second target). Several Techneins were identified. FIGS. 2A-2C. These Techneins contained amino acid and non-amino acid building blocks. FIG. 2D.

The binding affinities for each Technein were determined by microscale thermophoresis (MST), using Spike protein RBD domain as a target. A subset of the results is summarized in Table 4. MST experiments were performed on a Monolith NT.115pico (NanoTemper Technologies GmbH, Munich, Germany). Measurements were performed at room temperature, in triplicate with incubation periods of 15, 30 and 45 minutes. Binding affinities were obtained from a 16-point, two-fold dilutions series with ligand starting concentration at 1 μM and target concentration at 5 nM. SRBD protein was labeled using Nanotemper Monolith 2nd Generation Protein Labeling Kits (Maleimide-647-dye. The buffer for the SRBD contained 20 20 mM HEPES pH 7.4, 150 mM NaCl, 10 mM MgCl2, and 0.05% Tween-20. Triplicate data was analyzed using MO.AffinityAnalysis software (NanoTemper Technologies GmbH).

TABLE 4
Binding Affinities for Exemplary Techneins

| Hit ID | Target | KD (nM) |
|---|---|---|
| 16838-89-1 | SRBD - wt | 36 |
| 16838-89-2 | SRBD - wt | 18 |
| 16838-89-3 | SRBD - wt | 240 |
| 16838-89-4 | SRBD - wt | 123 |
| 16838-89-5 | SRBD - wt | 70 |
| 16838-89-6 | SRBD - wt | 210 |
| 16838-89-7 | SRBD - wt | 85 |
| 16838-89-8 | SRBD - wt | 96 |
| 16838-89-9 | SRBD - wt | 199 |
| 16838-89-10 | SRBD - wt | 92 |
| 16838-89-11 | SRBD - wt | 310 |
| 16838-89-12 | SRBD - wt | No binding |
| 16838-89-13 | SRBD - wt | 235 |
| 16838-89-14 | SRBD - wt | 216 |
| 16838-89-15 | SRBD - wt | 200 |
| 16838-89-16 | SRBD - wt | 119 |
| 16838-89-17 | SRBD - wt | 185 |
| 16838-89-18 | SRBD - wt | No binding |
| 16838-89-19 | SRBD - wt | No binding |
| 16838-89-20 | SRBD - wt | No binding |

A subset of Techneins were identified as lead hits and tested against different Spike proteins in biolayer interferometry (BLI). The results are summarized in Table 5. BLI analysis was performed using a Sartorius Octet instrument. Techneins at concentrations ranging from 1-250 nM in buffer (20 mM MOPS, 25 mM KCl, 0.02% Tween-20), were captured using a SAX biosensor dip and read for 600 seconds. The loaded sensors were quenched with 25 g/mL biocytin (MOPS) for 250 seconds. The baseline was then measured in MOPS buffer alone. Sensors were then dipped into spike RBD analyte (MOPS buffer) at 250 nM to 1.029 nM in 3-fold dilution series for 600 seconds. The sensors were then dipped in buffer along for 800 seconds to allow for dissociation. Data were analyzed using the HT11.1 analysis software with Savitzky-Golay filtering to reduce the high frequency noise. A global 1:2 fitting was performed to determine the association rate constant (ka) and dissociation rate constant (kd) and kinetic affinity (KD).

TABLE 5
Binding Affinities for Selected Techneins

| Hit ID | Target | KD (nM) |
|---|---|---|
| 16838-89-7 | SRBD - wt | 20 |
| 16838-89-7 | SRBD - omicron | 7 |
| 16838-89-7 | Full spike - wt | 7 |
| 16838-89-8 | SRBD - wt | 16 |
| 16838-89-8 | SRBD - omicron | 4 |
| 16838-89-8 | Full spike - wt | 2 |
| 16838-89-8-scrambled | SRBD - wt | 9 |
| 16838-89-8-scrambled | SRBD - omicron | 3 |
| 16838-89-8-scrambled | Full spike - wt | 0.8 |

### Example 2

### Identification of New Techneins Against Herceptin Antibody

A DAAP2 and a DHQ11 library were screened for Techneins that bound the Herceptin antibody. A number of hits were identified. FIGS. 3A and 3B. These Techneins also comprise amino acid and non-amino acid monomers. FIGS. 3C and 3D.

The binding affinities to Herceptin were tested in BLI. The results are summarized in Table 6.

TABLE 6
Binding Affinities for Selected Techneins Against Herceptin

| Hit ID | KD (nM) |
|---|---|
| 16989-11-17 | 6 |
| 16910-59-7 | 150 |
| 16989-3-2 | 472 |
| 16989-3-1 | 477 |
| 16989-3-14 | 483 |
| 16989-3-13 | 559 |
| 16910-59-5 | 653 |
| 16989-3-6 | 730 |
| 16989-3-17 | 1340 |
| 16989-3-19 | 1360 |
| 16989-3-9 | 1360 |
| 16910-59-6 | 4650 |

### Example 3

### Repurposing Agent Against Spike Protein

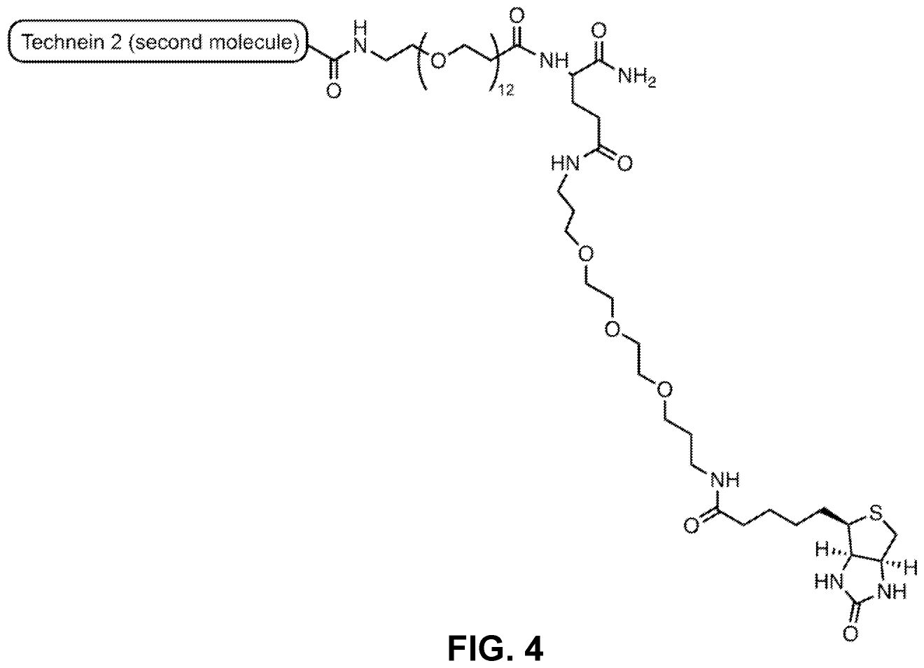

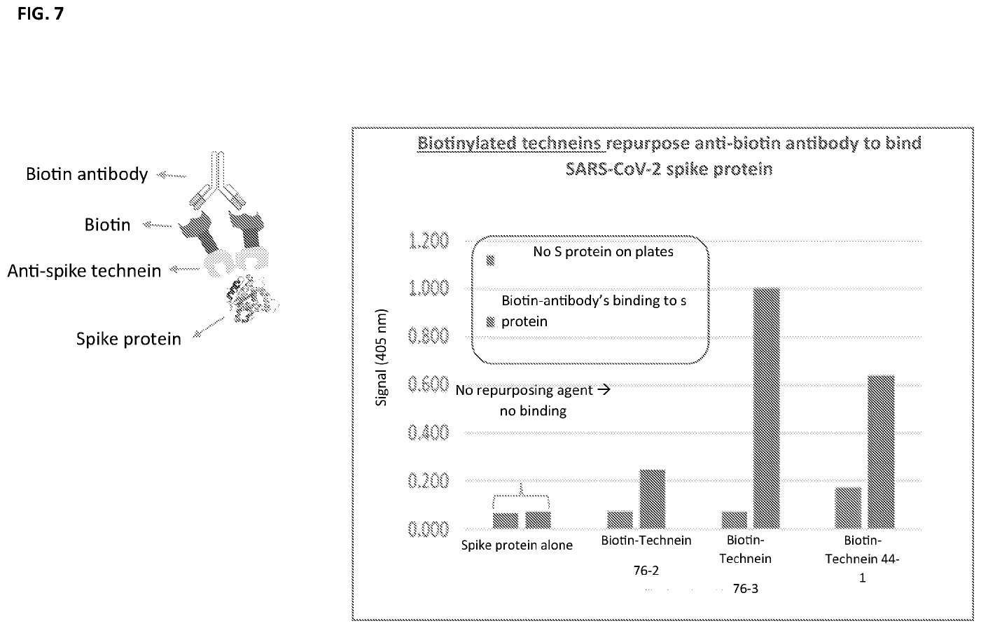

Anti-Spike Techneins (second molecules) were conjugated to biotin (first molecule) via a linker added to their C-terminus. FIG. 4. As shown later, these Techneins may be used to repurpose an anti-biotin antibody (first target) into an anti-Spike protein antibody-Technein conjugate (second target). FIG. 7.

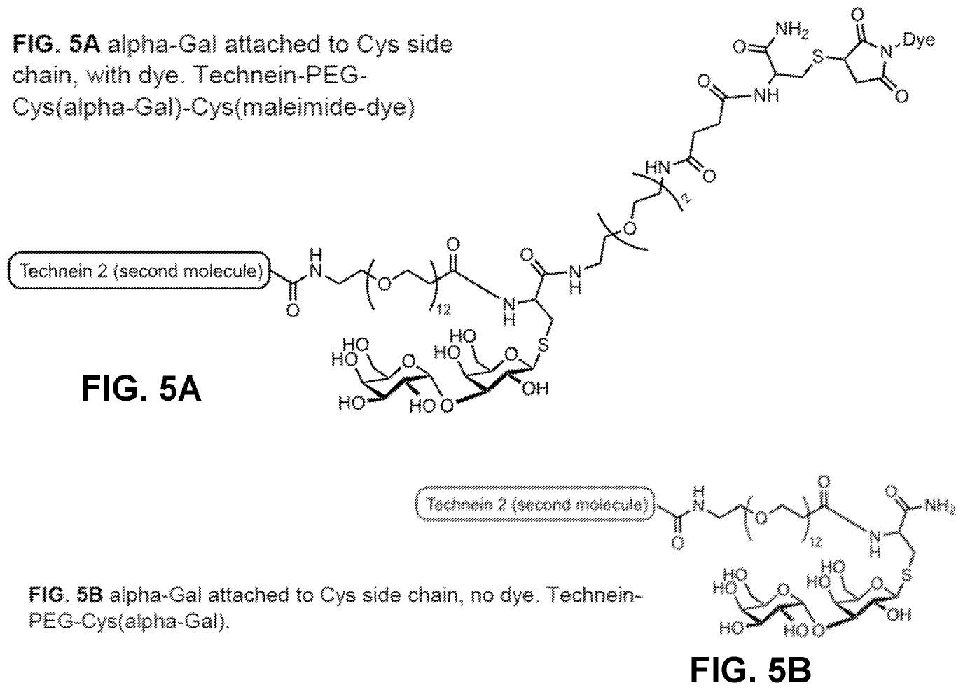

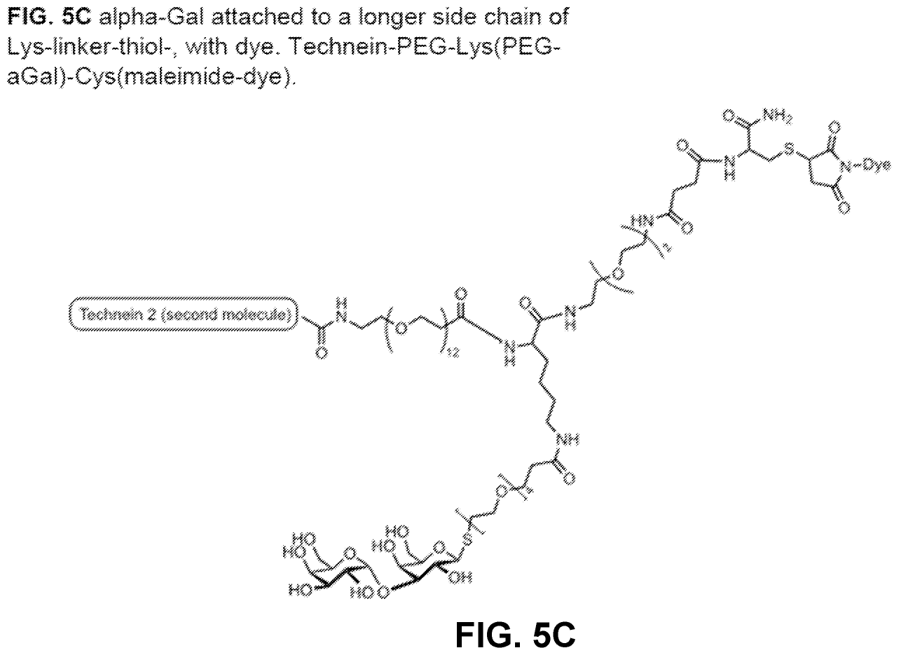

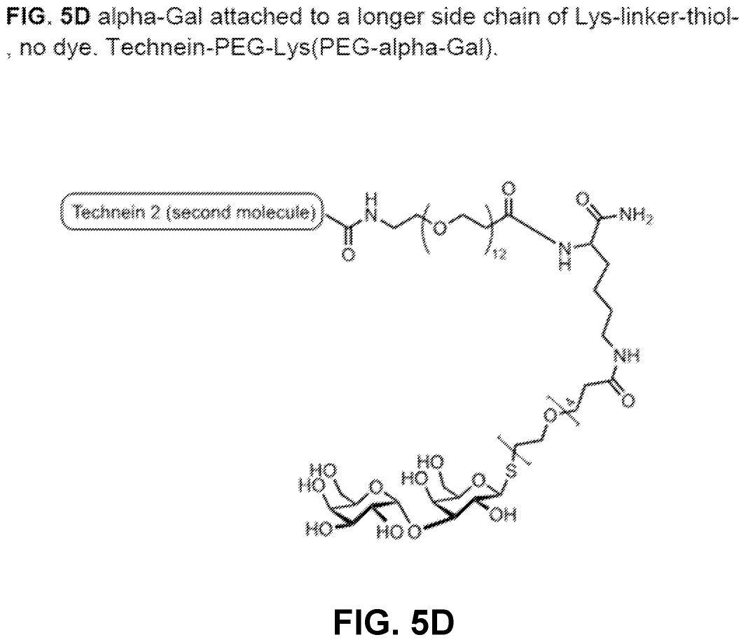

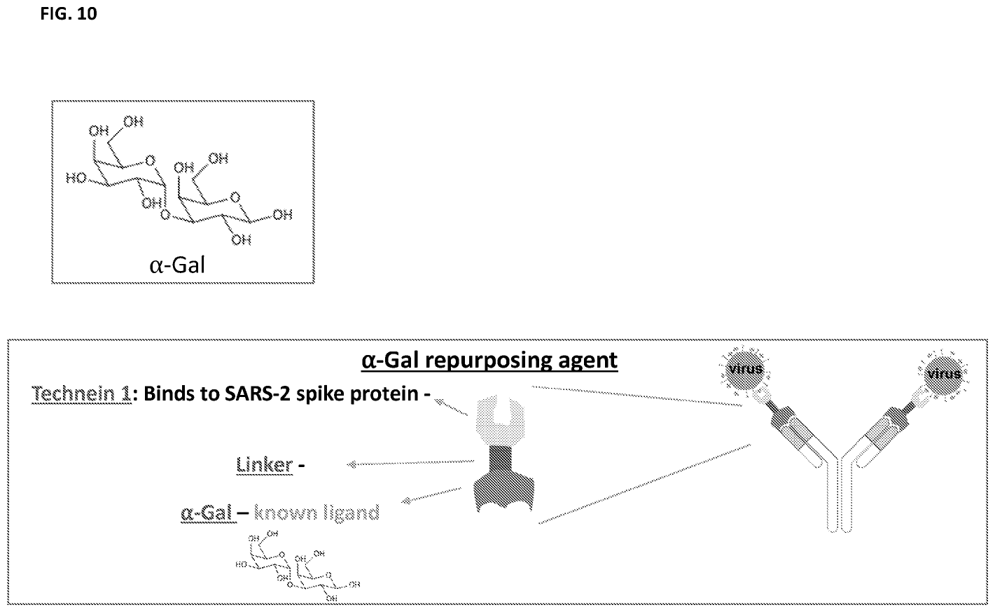

Anti-Spike Techneins (second molecules) were conjugated to alpha-Gal (also known as α-Gal) (first target, which is also a first molecule) via a linker comprising PEG linked to a Cys side chain, with a dye (FIG. 5A) or without a dye (FIG. 5B). Other anti-Spike Technein-α-Gal conjugates are described in FIGS. 5C and 5D. As shown later, this synthetic compound serves to repurpose an endogenous anti-alpha-Gal (first target) antibody against anti-Spike protein (second target). FIG. 10.

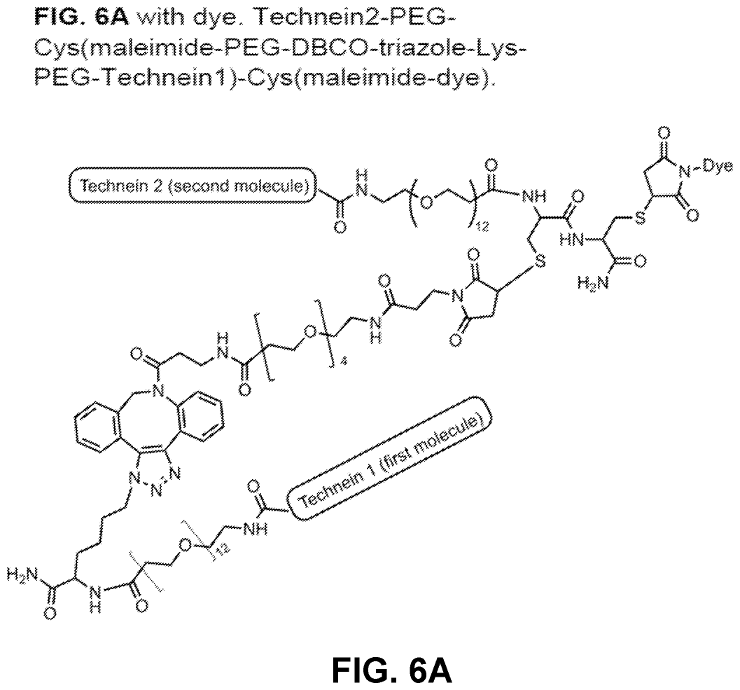

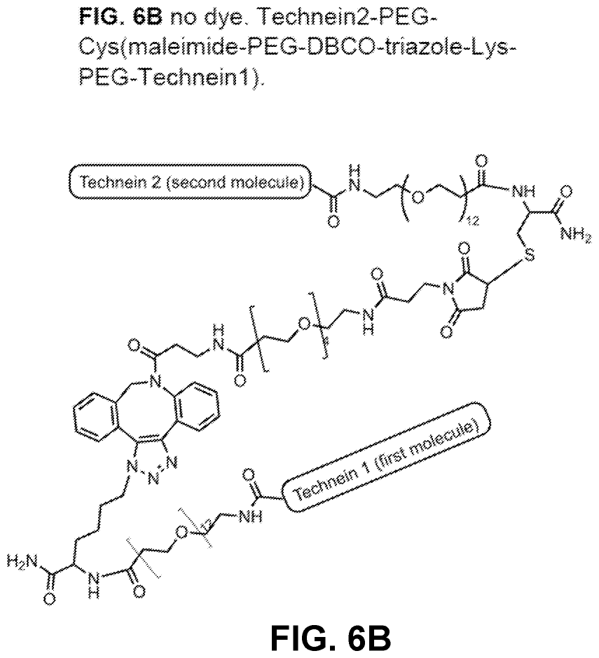

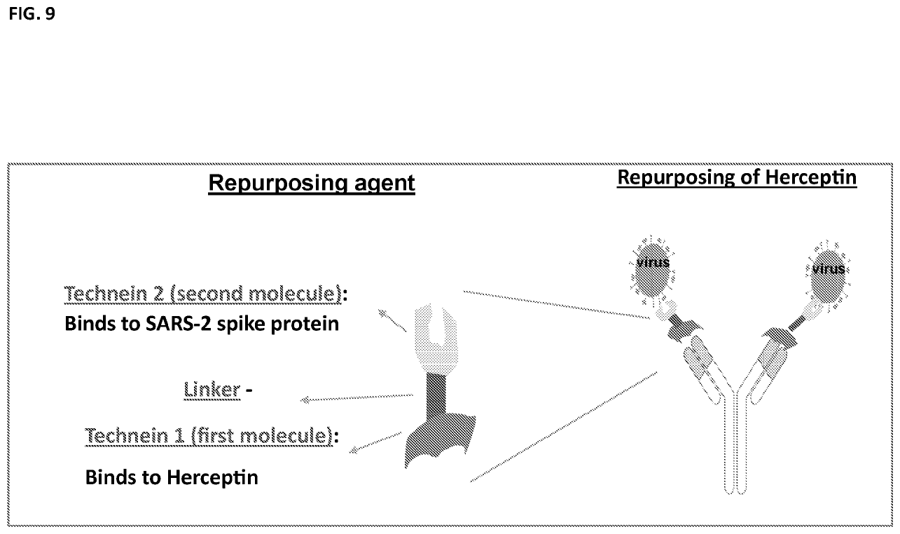

Anti-Spike Techneins (second molecules) were conjugated to anti-Herceptin Techneins (first molecules) via a linker, with (FIG. 6A) or without (FIG. 6B) dye. As shown later, this synthetic compound serves to repurpose an anti-Herceptin antibody (trastuzumab) into an anti-spike antibody. FIG. 9.

### Example 4

### Antigen (SARS-COV2 SRBD) Bead-Bound Phagocytosis (Antibody-Dependent Cellular Phagocytosis, ADCP) Assays Analyzed by Flow Cytometry

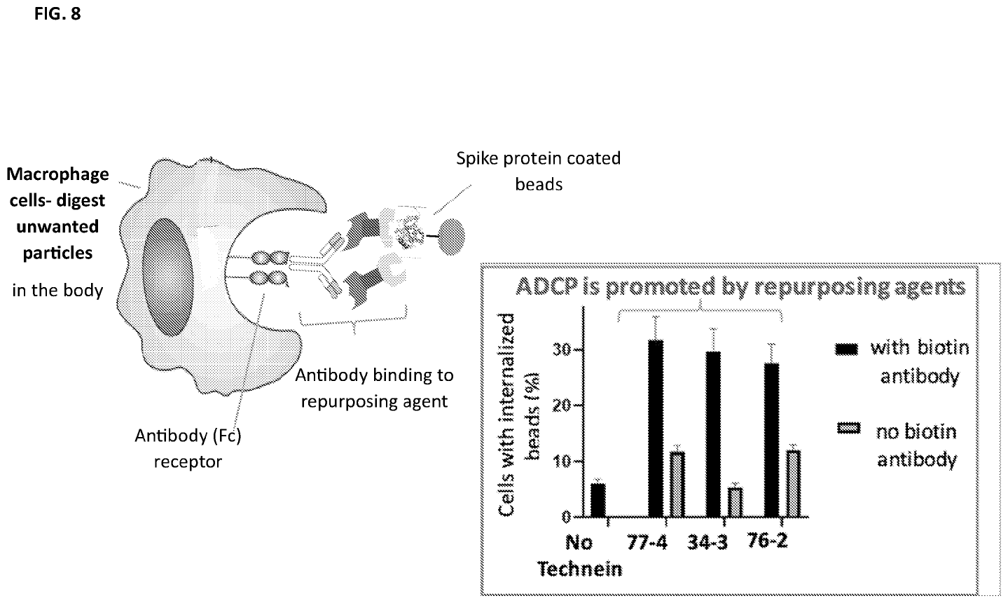

SARS-COV-2 SRBD coated beads were prepared by antigen adsorption. A total of 130 million carboxylated latex fluorescent beads 1 μm in diameter were incubated overnight with a concentration of 40 μg/ml of protein in 1 ml of PBS at 4° C. Beads were subsequently washed twice with PBS plus 1% BSA and resuspended in RPMI medium. Three different biotinylated anti-SRBD Techneins were preincubated with mouse anti-biotin Ab or mock medium for 30 minutes at 37° C. to form the complex. The Technein-Ab complex (anti-biotin Ab repurposed against SRBD) then added to the prepared SRBD beads and incubated for 1 hr at 37° C. Raw264.7 cells were then co-incubated for 1 h at 37° C. with the Technein/anti-biotin Ab coated beads for phagocytosis. The cells were stained afterward extracellularly with rabbit anti-SRBD Ab and goat anti-rabbit AF647. The phagocytic efficiency (Y-axis in the graph) was calculated with percentages of Raw264.7 cells with beads and lack of extracellular anti-rabbit AF647 staining. (Technein 77-4:16877-77-4; 34-3:16877-34-3, 76-2:16788-76-2). Data represent means±SEM (n=3). ADCP is promoted by the repurposing agents. FIG. 8.

### Example 5

### Binding to Both Anti-Alpha-Gal Antibody and SARS-COV2 Antigens (SARS-COV2 SRBD or Omicron S1/S2 ECD) by the AB Repurposing Techneins in a Sandwich Enzyme-Linked Immunosorbent Assay (ELISA)

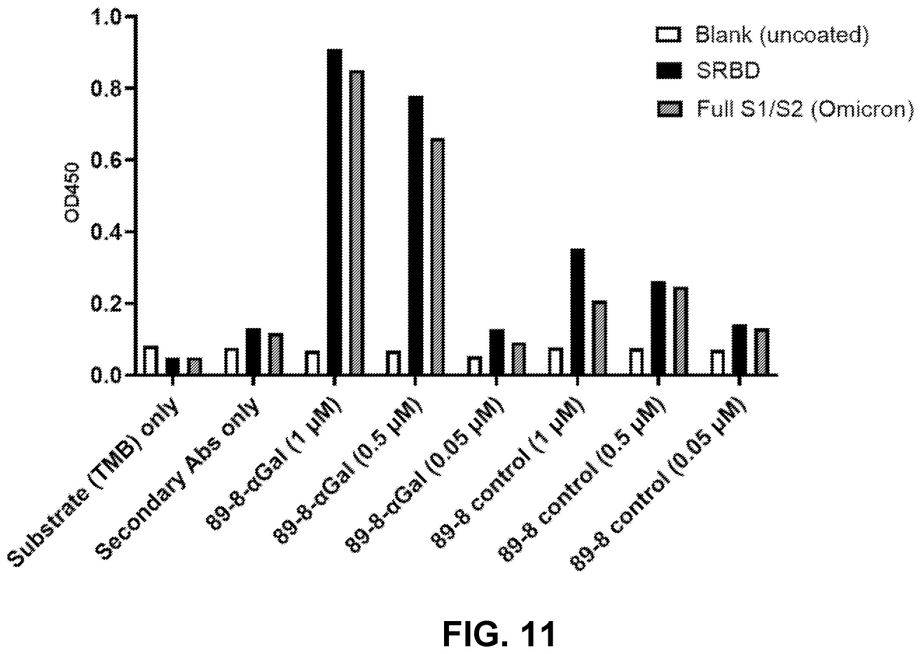

ELISA microplates were coated with SARS-Cov2 SRBD or Omicron S1/S2 ECD by incubating overnight with a concentration of 10 μg/ml of protein in 50 μl/well of PBS at 4° C. The plates were subsequently washed four times with PBS plus 0.05% Tween 20 (PBST) and then the reactions were blocked 2 hrs with PBST plus 1% BSA. The repurposing SARS-COV-binding Technein (89-8-aGal) and the unconjugated control SARS-COV-binding Technein (89-8) were added at the indicated concentrations in 50 μl/wells of 10 mM HEPES/1% DMSO/0.05% Tween 20 and incubated for 1 hr at RT. After wash, the 50 μl/well of the mouse anti-alpha-Gal antibody (1:1000, m86, Absolute Antibody) was added and further incubated for 1 hr at RT. The ELISA signals were developed with Horseradish Peroxidase (HRP) conjugated anti-mouse IgG and 3,3′,5,5′-Tetramethylbenzidine (TMB) substrate. (Blank: negative control without any coated antigen; secondary only: negative control without repurposing Technein or control Technein). The results are shown in FIG. 11. The repurposing agent alpha Gal-89-8 exhibited antibody repurposing of alpha Gal antibodies, enabling them to bind to the spike protein, including both the wild-type SRBD and the full spike omicron. As a negative control, compound 89-8 without alpha-Gal was utilized, demonstrating significantly lower levels of antibody repurposing.

### Example 6

### Adcp Assay with the Herceptin Antibody Repurposing Agent Alpha-Gal-Technein 89-8

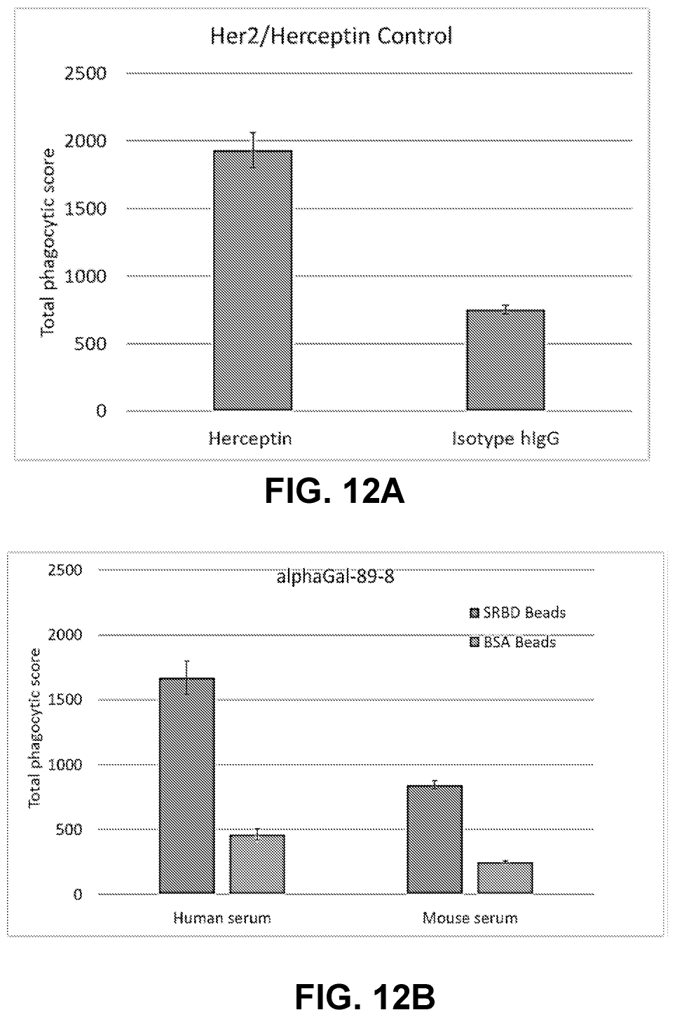

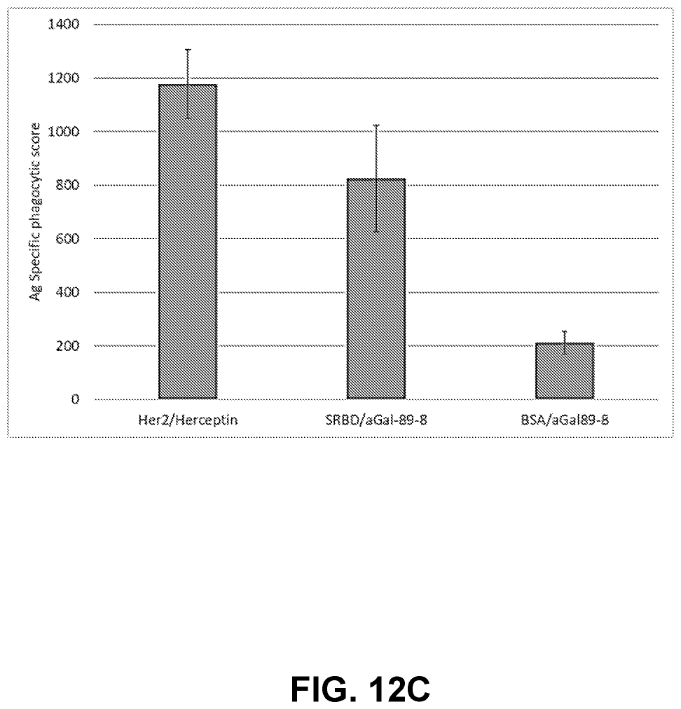

2.8 μm Streptavidin labeled magnetic Dynabeads™ M-280 (ThermoFisher) were washed in PBS containing 2% BSA (PBS 2% BSA) and incubated with 0.2 ug of biotinylated-HER2 (FIG. 12A), or biotinylated-SRBD (FIG. 12B), or biotinylated-BSA (FIG. 12B), each mixed with 0.2 ug/ml of Biotin-FTIC in 250 μl of PBS 2% BSA overnight at 4° C. Beads were washed in PBS 2% BSA to remove unbound protein and dye and resuspended in RPMI 1640 containing 10% FCS at a 1:100 dilution. Then:

(A) 20 μl of biotinylated-HER2 coated beads were incubated in triplicated wells with 0.2 μg/ml HER2-specific Ab Herceptin in RPMI 1640 containing 10% FCS for 30 min at 37° C. As a negative control, in parallel, another 20 μl of biotinylated-HER2 coated beads were incubated in triplicated wells with 0.2 ug/ml irrelevant human hIgG in RPMI 1640 containing 10% FCS also for 30 min at 37° C. FIG. 12A.

(B) The repurposing agent alpha-Gal-89-8 (200 nM) was pre-incubated with 1:10 diluted human serum (=human serum preparation) or mouse serum (=mouse serum preparation) for 1 hour at room temperature (unlike human serum, the mouse serum lacks alpha Gal antibodies). 20 μl of biotinylated-SRBD coated beads were incubated in triplicated wells with the human serum preparation in RPMI 1640 containing 10% FCS for 30 min at 37° C. As a negative control, in parallel, 20 μl of biotinylated-BSA coated beads were treated the same way as the biotinylated-SRBD coated beads, i.e., incubated in triplicated wells with the human serum preparation in RPMI 1640 containing 10% FCS for 30 min at 37° C. In parallel, two more negative controls were set up utilizing 20 μl of biotinylated-SRBD coated beads and 20 μl of biotinylated-BSA coated beads each incubated in triplicated wells with the mouse serum preparation in RPMI 1640 containing 10% FCS for 30 min at 37° C. FIG. 12B.

(A+B) 4×10\hat{ }4 effector monocytes THP-1 were then added to all opsonized beads. Plates were spun for 1 min at 300 g to promote contacts and further incubated for 3 h at 37° C. Cells were fixed in 4% paraformaldehyde and were immediately acquired on a A3 Symphony flow cytometer (BD Biosciences). Total phagocytic score was determined by the percentage of cells with beads, multiplied by their MFI. FIG. 12A, B.

(C) To calculate antigen specific phagocytic score for the biotinylated-HER2 coated beads shown in A, total phagocytic score obtained in the presence of the irrelevant hIgG was deducted from phagocytic score obtained in the presence of HER-2 specific Ab Herceptin. FIG. 12C.

(C) To calculate antigen specific phagocytic score for the biotinylated-SRBD coated beads and for the biotinylated-BSA coated beads shown in B, total phagocytic score obtained in the presence of the mouse serum preparation was deducted from total phagocytic score obtained in the presence the human serum preparation. FIG. 12A-C.

### Example 7

### SARS-COV-2 Spike Protein Binding by Technein Monomer and Dimer

Technein binding to SARS-COV-2 SRBD (wild type) was demonstrated by ELISA assay. Recombinant SRBD protein (Sino Biological) was immobilized onto a 96-well ELISA plane in 1% BSA, 0.1% Tween-20 in PBS pH 7.4. Wells were thoroughly washed with buffer and then biotinylated technein 76-3 monomer (100 nM) and biotinylated technein dimer 16877-9-4 (100 nM) were applied to the wells along with no Technein background controls. After washing, the wells were treated with streptavidin-HRP to measure the degree of binding with an absorbance readout at 405 nm. FIG. 13.

### Example 8

### Method for Rapid Vaccination by Antibody Repurposing

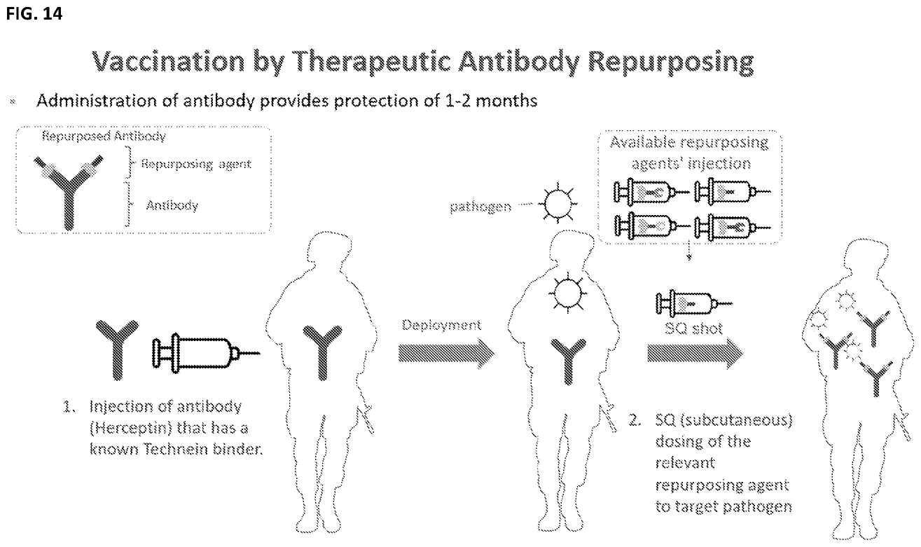

Antibody repurposing enable the production of rapid immunity to a pathogen by first administering a therapeutic antibody that binds to a known antigen or hapten. For example, this could be the FDA-approved antibody drug trastuzumab (Herceptin) that was developed to bind to the protein HER2. Administration of a bifunctional repurposing agent (synthetic compound of the disclosure) that binds to this antibody via a first molecule and additionally binds to a second target on a viral pathogen, such as the SARS-COV-2 spike protein, via the second molecule, will target the original antibody to the pathogen and produce an immunological response to clear the pathogen from circulation. This method enables a common antibody component to be used with multiple different repurposing agents that are specific for various types of pathogens causing human disease. FIG. 14.

We claim:

1. A synthetic compound comprising at least one unit, each unit comprising:
(i) a first molecule that binds to an antibody, or to a target-binding fragment thereof; wherein the antibody originally binds to a first target; and
(ii) a second molecule that binds to a second target;
wherein the first target and the second target are different, and wherein the first molecule and the second molecule are covalently linked, directly or through a linker.

2. The synthetic compound of claim 1, wherein the first molecule comprises the first target, an epitope thereof, an antibody-binding fragment thereof, or a first synthetic polymer; and the second molecule comprises a second synthetic polymer that binds to a second target, wherein the first and second synthetic polymers comprise amino acid monomers and/or non-amino acid monomers.

3. The synthetic compound of claim 1, wherein the first molecule non-covalently binds to one or more complementarity determining regions (CDRs) of the antibody.

4. The synthetic compound of claim 1, wherein the antibody has been approved by one or more agencies selected from Federal Drug Administration (FDA), European Medicines Agency (EMA), SwissMedic, China Food and Drug Administration (CFDA), and Japanese Pharmaceuticals and Medical Devices Agency (PMDA).

5. The synthetic compound of claim 4, wherein the antibody is selected from Trastuzumab (Herceptin), Adalimumab (Humira), Bevacizumab (Avastin), Rituximab (Rituxan/MabThera), Infliximab (Remicade), or any other antibody from Table 1.

6. The synthetic compound of claim 1, wherein the antibody is an endogenous antibody.

7. The synthetic compound of claim 6, wherein the first target and the first molecule are both alpha-Gal.

8. The synthetic compound of claim 1, wherein the targets are selected from proteins, sugars, carbohydrates, nucleic acids, lipids, and combinations thereof.

9. The synthetic compound of claim 1, wherein the targets are viral, tumor-specific (e.g., tumor-associated antigens), tissue-specific, cell-specific, bacterial, fungal, and combinations thereof.

10. The synthetic compound of claim 1, wherein the first target is Human Epidermal growth factor Receptor 2 (HER2) and/or the second target is a viral protein (including, but not limited to SARS-COV-2 spike protein).

11. The synthetic compound of claim 2, wherein the amino acids comprise L-amino acids, D-amino acids, beta-amino acids, gamma-amino acids, or combinations thereof, and the non-amino acid monomers comprise PAM, DhqF, DhqB, DhqO, DhqY, DhqE, N-substituted glycines, triazines, pyrimidines, or combinations thereof.

12. The synthetic compound of claim 1, wherein the linker comprises Polyethylene glycol (PEG), poly-glycine sequences, peptides, alkyl chains, cysteines, lysines, glutamines, maleimides, dibenzyclooctanes, or combinations thereof.

13. The synthetic compound of claim 1, wherein at least one unit comprises multiple first and/or second molecules, wherein:
i. the multiple first molecules are covalently bound to each other directly or through a linker; and/or
ii. the multiple second molecules are covalently bound to each other directly or through a linker;
preferably, wherein the linker(s) comprise PEG, Lys, Gln, or combinations thereof.

14. The synthetic compound of claim 1, wherein the first molecule and/or second molecule binds to the antibody or second target, respectively, with an affinity below 500 nM, preferably below 200 nM, more preferably below 100 nM.

15. The synthetic compound of claim 1, wherein the first molecule and/or second molecule does not bind significantly to any other target, also referred to as being specific for the first target or second target, respectively.

16. A conjugate comprising an antibody, or antigen-binding fragment thereof, non-covalently bound to at least one synthetic compound of any one of claims 1 to 15, wherein the conjugate is also referred to as a repurposed antibody, or repurposed fragment thereof.

17. The conjugate of claim 16, wherein the antibody comprises a light chain and a heavy chain immunoglobulin variable region and constant regions.

18. The conjugate of claim 16, wherein the antibody, or antigen-binding fragment thereof, is selected from monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab, Fab′, F(ab′)2, and Fv fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments of any of the above.

19. The conjugate of any one of claims 16 to 18 and/or synthetic compound of any one of claims 1 to 15, wherein the antibody that binds to the first molecule, the conjugate (repurposed antibody), and/or the synthetic compound is an immunotherapeutic agent.

20. A method of repurposing an antibody that binds to a first target, or a first target-binding fragment thereof, by changing its target from a first target to a second target, the method comprising non-covalently conjugating the antibody, or first target-binding fragment thereof, with at least one synthetic compound of any one of claims 1 to 15, wherein the second target is also referred to as the repurposed target and wherein the conjugate is also referred to as the repurposed antibody.

21. A method of treating a disorder with a repurposed antibody in a subject in need thereof, comprising administering to the subject an effective amount of the synthetic compound of claims 1 to 15 (or a part thereof), the antibody of any one of claims 1 to 15, and/or the repurposed antibody of anyone of claims 16 to 19.

22. The method of claim 21, wherein the disorder is an infectious disorder caused by a pathogen that comprises the second target or a cancer expressing the second target.

23. The method of claim 22, wherein the pathogen is a virus (including, without limitation, the SARS-COV-2 coronavirus).

24. The method of any one of claims 20 to 23, wherein the antibody is an endogenous antibody.

25. A method for treating a disease caused by a pathogen in a subject in need thereof comprising:
(i) administering to the subject an antibody that binds to the first molecule of a synthetic compound according to any one of claims 1 through 15, or confirming that the subject carries an endogenous antibody that binds to the said compound;
(ii) exposing or having exposed the subject to a pathogen, an epitope of the pathogen, and/or a part of the pathogen; and
(iii) administering the synthetic compound of (i) to the subject, wherein the pathogen comprises the second target of the synthetic compound.

26. The method of claim 25, wherein the pathogen causes a disease selected from a viral disease, a bacterial disease, a fungal disease, or a tick-associated disease.

27. A method of ameliorating, treating, or reducing the incidence of a cancer/tumor/malignancy in a subject in need thereof, preferably, wherein the method assists or boost the immune system in killing or eradicating cancerous cells, wherein the method comprises administering a therapeutic effective amount of one or more conjugates/repurposed antibodies, synthetic compounds, or antibodies of any one of claims 1 to 19 to the subject, preferably one which invokes an active (or achieve a passive) immune response to destroy cancerous cells.

28. A method of ameliorating, treating, or reducing the incidence of a cancer/tumor/malignancy in a subject in need thereof, preferably, wherein the method assists or boost the immune system in killing or eradicating cancerous cells, wherein the method comprises administering to the subject a therapeutic effect amount of a CAR-T cell, T cell, and/or Tumor Infiltrating Lymphocyte contacted with or bound to an immunotherapeutic compound of any one of claims 1 to 19, preferably, wherein the cells invoke an active (or achieve a passive) immune response to destroy cancerous cells.

29. A method of using a therapeutic effective amount of conjugates/repurposed antibodies, synthetic compounds, or antibodies, of any one of claims 1 to 19, or immunotherapeutic cells (including using autologous and/or heterologous cells or immortalized cell lines) conjugated or contacted with a compound or conjugate of any one of claims 1 to 19, in vivo or ex vivo as an adoptive immunotherapies for treating a cancer, in a subject in need thereof, comprising using or administering one or more conjugates/repurposed antibodies, synthetic compounds, antibodies of any one of claims 1 to 19 to a subject and/or contacting the immunotherapeutic cells with one or more conjugates/repurposed antibodies, synthetic compounds, antibodies cells, and/or nucleic acids of any one of claims 1 to 19.

30. A composition comprising one or more conjugates/repurposed antibodies, synthetic compounds, and/or antibodies of one of claims 1 to 19, and/or CAR-T cells, T cells, or TILs contacted with or conjugated with a compound of any one of claims 1 to 19, preferably wherein the composition is a pharmaceutical composition.

31. An immunotherapeutic compound of anyone of claims 1 to 19, or composition of claim 30, used in a cancer immunotherapy, wherein the immunotherapeutic compound or composition comprises a repurposed antibody, a fragment thereof, and/or synthetic compound of the disclosure, and/or a CAR-T cell, T cell, and/or Tumor Infiltrating Lymphocyte contacted with or conjugated with a compound of any one of claims 1 to 19.

32. Use of any one of the synthetic compounds of any one of claims 1 to 15, conjugates of any one of claims 16 to 19, compositions comprising the same, and/or CAR-T cells, T cells, or TILs contacted with or conjugated with a compound of any one of claims 1 to 19 in the treatment of a disease or disorder.

33. The use of claim 32, wherein the disease or disorder is an infectious disease or disorder or a cancer, or any other disease disorder described in the specification.

34. A synthetic compounds of any one of claims 1 to 15, conjugates of any one of claims 16 to 19, compositions comprising the same, and/or CAR-T cells, T cells, or TILs contacted with or conjugated with a compound of any one of claims 1 to 19 for use in the manufacture of a medicament in the treatment of a disease or disorder, preferably, wherein the disease or disorder is an infectious disease or disorder or a cancer, or any other disease disorder described in the specification.


SEQUENCE LISTING (61 sequences)

SEQ ID NO: 1 - protein, 4 aa, synthetic construct
  MOD_RES 2: note = beta-cyclopropyl-L-alanine
AAVA

SEQ ID NO: 2 - protein, 5 aa, synthetic construct
  SITE 1..4: note = D-amino acid
SFPEL

SEQ ID NO: 3 - protein, 5 aa, synthetic construct
  SITE 2..4: note = D-amino acid
GVPRA

SEQ ID NO: 4 - protein, 5 aa, synthetic construct
  SITE 1..4: note = D-amino acid
PSERG

SEQ ID NO: 5 - protein, 5 aa, synthetic construct
  SITE 1..4: note = D-amino acid
YTPRA

SEQ ID NO: 6 - protein, 5 aa, synthetic construct
  SITE 1: note = D-amino acid
  SITE 3..4: note = D-amino acid
PGRWG

SEQ ID NO: 7 - protein, 5 aa, synthetic construct
  SITE 1..4: note = D-amino acid
LPRTG

SEQ ID NO: 8 - protein, 5 aa, synthetic construct
  SITE 1..4: note = D-amino acid
YHPWV

SEQ ID NO: 9 - protein, 5 aa, synthetic construct
  SITE 2..4: note = D-amino acid
GVPRA

SEQ ID NO: 10 - protein, 5 aa, synthetic construct
  SITE 1..4: note = D-amino acid
HSPRV

SEQ ID NO: 11 - protein, 5 aa, synthetic construct
  SITE 1..4: note = D-amino acid
VKTVG

SEQ ID NO: 12 - protein, 5 aa, synthetic construct
  SITE 1..4: note = D-amino acid
ETHWL

SEQ ID NO: 13 - protein, 5 aa, synthetic construct
  SITE 1..4: note = D-amino acid
FYSAA

SEQ ID NO: 14 - protein, 5 aa, synthetic construct
  SITE 1..4: note = D-amino acid
SPHEF

SEQ ID NO: 15 - protein, 5 aa, synthetic construct
  SITE 1..4: note = D-amino acid
TYKVL

SEQ ID NO: 16 - protein, 5 aa, synthetic construct
  SITE 2..4: note = D-amino acid
GPRSF

SEQ ID NO: 17 - protein, 5 aa, synthetic construct
  SITE 1..4: note = D-amino acid
VKTVG

SEQ ID NO: 18 - protein, 5 aa, synthetic construct
  SITE 1..4: note = D-amino acid
PRVNV

SEQ ID NO: 19 - protein, 5 aa, synthetic construct
  SITE 1..4: note = D-amino acid
EHRTV

SEQ ID NO: 20 - protein, 5 aa, synthetic construct
  SITE 1..4: note = D-amino acid
LPESF

SEQ ID NO: 21 - protein, 5 aa, synthetic construct
  SITE 2..4: note = D-amino acid
GVPRA

SEQ ID NO: 22 - protein, 5 aa, synthetic construct
  SITE 2..4: note = D-amino acid
GPRSF

SEQ ID NO: 23 - protein, 5 aa, synthetic construct
  SITE 1..4: note = D-amino acid
NPLHL

SEQ ID NO: 24 - protein, 5 aa, synthetic construct
  SITE 1..4: note = D-amino acid
RFPVG

SEQ ID NO: 25 - protein, 5 aa, synthetic construct
  SITE 1..4: note = D-amino acid
PPSRV

SEQ ID NO: 26 - protein, 5 aa, synthetic construct
  SITE 1..4: note = D-amino acid
LPESF

SEQ ID NO: 27 - protein, 5 aa, synthetic construct
  SITE 2..4: note = D-amino acid
GVPRA

SEQ ID NO: 28 - protein, 5 aa, synthetic construct
  SITE 2..4: note = D-amino acid
GPRSF

SEQ ID NO: 29 - protein, 5 aa, synthetic construct
  SITE 1..4: note = D-amino acid
VKTVG

SEQ ID NO: 30 - protein, 5 aa, synthetic construct
  SITE 1..4: note = D-amino acid
PRVNV

SEQ ID NO: 31 - protein, 5 aa, synthetic construct
  SITE 1..4: note = D-amino acid
LPRTG

SEQ ID NO: 32 - protein, 5 aa, synthetic construct
  SITE 1..4: note = D-amino acid
YHPWV

SEQ ID NO: 33 - protein, 5 aa, synthetic construct
  SITE 2..4: note = D-amino acid
GVPRA

SEQ ID NO: 34 - protein, 5 aa, synthetic construct
  SITE 2..4: note = D-amino acid
GPRSF

SEQ ID NO: 35 - protein, 5 aa, synthetic construct
  SITE 1..4: note = D-amino acid
VKTVG

SEQ ID NO: 36 - protein, 5 aa, synthetic construct
  SITE 1..4: note = D-amino acid
PRVNV

SEQ ID NO: 37 - protein, 5 aa, synthetic construct
  SITE 1..4: note = D-amino acid
YPREG

SEQ ID NO: 38 - protein, 5 aa, synthetic construct
  SITE 1..4: note = D-amino acid
LPESF

SEQ ID NO: 39 - protein, 5 aa, synthetic construct
  SITE 2..4: note = D-amino acid
GVPRA

SEQ ID NO: 40 - protein, 5 aa, synthetic construct
  SITE 2..4: note = D-amino acid
GPRSF

SEQ ID NO: 41 - protein, 5 aa, synthetic construct
  SITE 1..4: note = D-amino acid
YTPRA

SEQ ID NO: 42 - protein, 5 aa, synthetic construct
  SITE 1..4: note = D-amino acid
AELKA

SEQ ID NO: 43 - protein, 5 aa, synthetic construct
  SITE 2..4: note = D-amino acid
GWHRA

SEQ ID NO: 44 - protein, 5 aa, synthetic construct
  SITE 1..4: note = D-amino acid
SPHEF

SEQ ID NO: 45 - protein, 5 aa, synthetic construct
  SITE 1..2: note = D-amino acid
  SITE 4: note = D-amino acid
PSGSA

SEQ ID NO: 46 - protein, 5 aa, synthetic construct
  SITE 2..4: note = D-amino acid
GPRSF

SEQ ID NO: 47 - protein, 5 aa, synthetic construct
  SITE 1..4: note = D-amino acid
YTPRA

SEQ ID NO: 48 - protein, 5 aa, synthetic construct
  SITE 1..4: note = D-amino acid
AELKA

SEQ ID NO: 49 - protein, 5 aa, synthetic construct
  SITE 1..4: note = D-amino acid
FYSAA

SEQ ID NO: 50 - protein, 5 aa, synthetic construct
  SITE 1..4: note = D-amino acid
YHPWV

SEQ ID NO: 51 - protein, 5 aa, synthetic construct
  SITE 1..2: note = D-amino acid
  SITE 4: note = D-amino acid
PSGSA

SEQ ID NO: 52 - protein, 5 aa, synthetic construct
  SITE 2..4: note = D-amino acid
GPRSF

SEQ ID NO: 53 - protein, 5 aa, synthetic construct
  SITE 1..4: note = D-amino acid
PKYRF

SEQ ID NO: 54 - protein, 5 aa, synthetic construct
  SITE 1..4: note = D-amino acid
RYTEA

SEQ ID NO: 55 - protein, 5 aa, synthetic construct
  SITE 1..4: note = D-amino acid
HYSEF

SEQ ID NO: 56 - protein, 5 aa, synthetic construct
  SITE 1..4: note = D-amino acid
LPESF

SEQ ID NO: 57 - protein, 5 aa, synthetic construct
  SITE 2..4: note = D-amino acid
GVPRA

SEQ ID NO: 58 - protein, 5 aa, synthetic construct
  SITE 2..4: note = D-amino acid
GPRSF

SEQ ID NO: 59 - protein, 5 aa, synthetic construct
  SITE 1..4: note = D-amino acid
VKTVG

SEQ ID NO: 60 - protein, 5 aa, synthetic construct
  SITE 1..4: note = D-amino acid
PRVNV

SEQ ID NO: 61 - protein, 5 aa, synthetic construct
  SITE 1..4: note = D-amino acid
YPREG